(19) United States
(12) Patent Application Publication
(10) Pub. No.: US 2025/0338857 A1
(43) Pub. Date: Nov. 6, 2025

(54) USE OF INTERFERING RNAS DIRECTED AGAINST THE CHOLINERGIC SYSTEM FOR CONTROLLING INSECT PESTS

(71) Applicant: UNIVERSITÉ D'ANGERS, ANGERS (FR)

(72) Inventors: Delphine GOVEN, Retiers (FR); Valérie RAYMOND, Bouchemaine (FR)

(21) Appl. No.: 18/867,718

(22) PCT Filed: May 19, 2023
(86) PCT No.: PCT/IB2023/055162

§ 371 Date: Nov. 20, 2024

(30) Foreign Application Priority Data
May 20, 2022 (FR) FR2204889

(51) Int. Cl. A01N 63/60; A01P 7/04; C12N 15/113; C12N 15/82

(52) U.S. Cl. CPC A01N 63/60; A01P 7/04; C12N 15/1138; C12N 15/8286; C12N 2310/113; C12N 2310/14

(57) ABSTRACT

An RNA interference method for controlling insect pests by inhibiting translation of messenger RNA (mRNA) of target genes in the insect's cholinergic system. The method includes preparing double-stranded RNA (dsRNA) or single-stranded antisense oligonucleotides specific to target mRNAs encoding nicotinic acetylcholine receptor subunits or auxiliary proteins, and administering an effective amount to induce mortality or increase sensitivity to insecticides. The target mRNAs include neuronal α and β subunits of nicotinic receptors and auxiliary proteins such as RIC-3, Lynx, NACHO, and UNC-50. Administration may be topical, oral, or through transgenic plants expressing the interfering RNA. The method is particularly effective against agricultural pests like Acyrthosiphon pisum and can restore sensitivity in insecticide-resistant insects. Compositions including the interfering RNA with transfection agents or solvents are provided, along with transgenic plants expressing the interfering RNA.

### CROSS-REFERENCE TO RELATED APPLICATIONS

This application is a national stage entry under 35 U.S.C. § 371 of International Patent Application No. PCT/IB2023/055162, filed May 19, 2023, which claims priority to French Patent Application No. FR2204889, filed May 20, 2022. The entire contents of each of the above applications are hereby incorporated by reference in their entirety.

### INCORPORATION BY REFERENCE OF SEQUENCE LISTING

The Sequence Listing, created on 13 Nov. 2024, having a file size of 254,736 bytes and file name “B33606WO_sequencelisting EN.xml” is hereby incorporated by reference in its entirety.

### BACKGROUND OF THE INVENTION

The present invention relates to the field of insect pest management. Giving particular consideration to the application of the French Ecophyto II+ plan, which targets reducing the use of phytosanitary products by 50%, there is a pressing need for a means of controlling insect pests. These insects are harmful from the viewpoint both of public health and for the proper conducting of human activities whether domestic or professional. The presence of cockroaches, bedbugs in a home just like the infestation of crops by aphids, leafhoppers, weevils, or others has a deleterious effect on human activity.

Modern agriculture is turning towards agro-ecological means for the control of insect pests. This, however, requires the development of novel, alternative techniques that are adapted accordingly.

Since the start of the XIX^{th} century, the world population has been constantly increasing from 7.5 billion in 2017 to a forecasted population of about 11 billion at the end of the XXI^{st} century. Given the problems of under-nutrition and malnutrition that exist worldwide, agriculture must face the major challenge of producing food resources in sufficient quantity and quality. Numerous pests causing considerable damage to plants can lead to quantitative losses and/or to changes in harvest quality. Among these pests, destructive insects cause between 20 and 40% of lost agricultural production each year.

With the reduction in the number of approved active substances, the controlling of insect pests is becoming more and more difficult. In addition, the unreasonable use of insecticides has led to the development of insect pests that are resistant to insecticides.

The inventors have particularly focused on the development of a strategy for controlling insect pests e.g. the pea aphid Acyrthosiphon pisum and the cockroach Periplaneta americana.

The pea aphid colonizes many cultivated leguminous plants such as peas, beans, broad beans, lentils, or alfalfa. Leguminous crops rank second in world production after cereals, representing about 13% of cultivated surface areas (Gepts et al., 2005). The pea aphid causes direct damage by sucking sap, but it is also capable of transmitting numerous viruses. To date 30 viruses transmitted by the pea aphid have been listed: bean mosaic virus, cucumber mosaic virus, etc.

Neonicotinoids are the insecticides that have been most used in agriculture over the last ten years. Although temporary approval was granted under the French law of 14 Dec. 2020 to protect crops of sugar beet threatened by massive infestations of aphids, against which current insecticide treatments are ineffective, the use of these neonicotinoids has been prohibited since 2018.

As a result, there are currently few chemical substances which can be used to control insect pests including crop destructive insects and more particularly aphids, making the management of these insects problematic since they have become adapted to and even resistant to these chemical insecticide treatments.

### SUMMARY OF THE INVENTION

The inventors provide a new strategy for controlling insect pests based on the use of interfering RNAs (RNAi) targeting the cholinergic system, as bioinsecticides or as agents synergizing the effect of an insecticide used in low dose. Indeed, the cholinergic system of insects plays a major role in their physiology.

Having regard to this major physiological role of acetylcholine (ACh) in insects, the cholinergic system represents a priority target for numerous families of insecticides e.g. carbamates and organophosphates which inhibit the activity of the degradation e of ACh, acetylcholinesterase; spinosyns, neonicotinoids, butenolides, sulfoximines and mesoionics targeting the cholinergic receptors of nicotinic type (nAChR).

Up until 2018, the use of neonicotinoids was the most efficient means of controlling destructive crop insects. These insecticides were widely used in agriculture as phytosanitary products, and as biocidal products by individuals or companies to control insects harmful for human and animal health.

Article 125 of the (French) law of 8 Aug. 2016 called the “Law on the Restoration of Biodiversity, Nature and the Countryside”, prohibits “the use of phytopharmaceutical products containing one or more active substances of the neonicotinoid family, and seeds treated with these products [ . . . ] as from Sep. 1^{st} 2018”, with some possible dispensations.

Since the prohibited use of neonicotinoids, chemical neurotoxic pest control has mainly been based on the use of pyrethroids. However the lesser efficacy of this class of insecticide on insect pests led to the temporary approval of the use of neonicotinoids for sugar beet crops, granted by the law of 14 Dec. 2020, until other solutions were found to protect these crops that are hugely threatened by aphids. Other known means for controlling aphids include the use of auxiliary insects such as Coccinella septempunctata, and of parasitoids such as Aphidius avenae, mechanical control with the installation of anti-insect nets, or trapping with the use of glue-coated panels.

The present invention is based on the use of the RNA interference technique which is a method specific to a given species (Whyard et al., 2009), and has allowed the development of a new specific strategy for the control of insect pests species, e.g. against the pea aphid Acyrthosiphon pisum, without affecting beneficial insects such as the European honey bee Apis mellifera.

Toxicity tests requested by the inventors were carried out by the Testapi laboratory to evaluate the acute toxicity on Apis mellifera L. bees, via oral route and contact toxicity, of the dsRNA preparation of the β2 sub-unit of the nicotinic receptor of Acyrthosiphon pisum. The study did not show any toxicity on bees of the preparation of the invention, and the study is considered to be valid since the mortality of the control population lay within the acceptable range and the reference toxic product (dimethoate) generated typical effects in the validated phases.

The RNA interference technique allows specific regulation of the expression of a protein. Once inside the cell, double stranded interfering RNAs (dsRNAs) are cleaved into small interfering RNAs of 21 to 25 nucleotides (siRNAs) by a RNase III called DICER. This ribonuclease transfers the siRNAs to the multienzyme complex RISC (RNA-induced silencing complex). While the sense strand of the siRNA called “passenger” is removed, the “guide” antisense strand complementary to the mRNA of the gene of interest directs the RISC complex towards the target mRNAs for degradation thereof, thereby preventing their translation.

The present invention targets the cholinergic system of the insect pest and more specifically the neuronal nicotinic sub-units forming the nicotinic receptors (nAChRs) and their auxiliary proteins.

These nAChRs, which the are targets of numerous insecticides, form part of transmembrane complexes located at the synapses and allow the rapid transmission of nervous information when activated by ACh binding. They are pentameric glycoprotein complexes belonging to the “Cys-loop” family which comprises different ionotropic receptors called “Ligand-Gated Ion Channel” (LGIC) and are permeable to different cations (Na⁺, Ca²⁺ and K⁺). These nAChRs composed of 5 sub-units can be homomeric (5 identical α sub-units) or heteromeric (5 different α or β sub-units). To date, analysis of the genome of various insects has allowed the identification of several nicotinic sub-units: 10 α sub-units from α1 to α10, and 10 β sub-units from β1 to β10, and the isoforms thereof (Jones et al., 2021; Dale et al., 2010). The α sub-units differ from the β sub-units through the presence of 2 adjacent cysteines in the amino-terminal part.

The composition of nAChRs in nicotinic sub-units is used to define their electrophysiological and pharmacological properties, as well as their sensitivity to insecticides.

For example, the sequencing of the A. pisum genome (International Aphid Genomics Consortium, 2010) allowed the identification of 11 genes encoding nicotinic sub-units (Dale et al., 2010). Among these, the sub-units α9, α10 and β2 are so-called “divergent” sub-units i.e. they show scarce sequence homology with known nicotinic sub-units in other insect species. The inventors have used the RNA interference technique to target one of these sub-units: the sub-unit Apisum β2, also called β2. Testing was necessary to determine the application mode of the interfering RNAs (dsRNAs). Therefore, a topical application of 400 ng of dsRNA per aphid was used in the experiments. After determining that this interfering RNA indeed causes a decrease in the number of transcripts encoding β2, the inventors were able to demonstrate that the aphids, which had received a topical application of these dsRNAs, showed an increase in mortality rate of about 6%, 31% and 51% at 24 h, 48 h and 72 h respectively, after the application.

Having regard to the fact that the composition of nAChRs in nicotinic sub-units determines their sensitivity to insecticides, the decrease in the expression of the mRNAs of the β2 sub-unit by the specific dsRNAs that was observed in the pea aphid, suggests a change in the composition of the nAChRs leading to modulated efficacy of an insecticide treatment.

The experimental results show that adult aphids which had absorbed the dsRNAs against the β2 sub-unit, and were then intoxicated with imidacloprid at a concentration close to LC₅₀, namely 5·10⁻³ μg/mL, have a slightly increased mortality (1.4 times) 72 h after acute intoxication, compared with control aphids. Therefore, the use of the specific dsRNAs inducing a modification in the composition of nAChRs would render insects more sensitive to the insecticide. This synergic effect is detailed below in the examples.

### BRIEF DESCRIPTION OF THE DRAWINGS

FIG. 1 shows a histogram quantifying the expression of transcripts of the nicotinic β2 sub-unit in the aphid Acyrthosiphon pisum at 2 h, 24 h and 72 h post-absorption of the dsRNAs (β2 or LacZ). Relative expression is normalized to the RPL7 gene (reference gene) following the 2-ΔΔCt method (*p<0.05 β2 vs LacZ for each time point, Mann-Whitney test, n=5 to 7).

FIG. 2 shows a histogram depicting corrected mortality rate of the aphids at 24 h, 48 h and 72 h after absorption of the LacZ or β2 dsRNAs (**p<0.01 vs LacZ, Mann-Whitney test, n=5 comprising 15 to 30 aphids per condition).

FIGS. 3A and 3B shows a graphical representation of the concentration-effect curve of the corrected mortality rate observed in aphid larvae, as a function of imidacloprid concentration at 48 h (FIG. 3A) and 72 h (FIG. 3B) (n=5 comprising 20 aphid larvae per concentration).

FIG. 4 shows a histogram depicting the corrected mortality rate of aphids having absorbed the dsRNAs (LacZ or β2) 72 h post-intoxication with imidacloprid (5·10⁻³ μg/mL). DMSO is the imidacloprid solvent used as control (**p<0.01 vs LacZ, Mann-Whitney test, n=3 to 5 comprising 15 to 30 aphids per n and per condition).

FIG. 5A shows a histogram quantifying the expression of the mRNAs of the nicotinic sub-units in the aphid A. pisum at 2 h post-absorption of the β2 or LacZ dsRNAs. Relative expression is normalized to the RPL7 gene (reference gene) following the 2-ΔΔCt method and the results are expressed as a ratio with the control condition (LacZ dsRNA, corresponding to the bar of value 1) (*p<0.05 β2 vs LacZ, **p<0.01, Mann-Whitney test).

FIG. 5B shows a histogram quantifying the expression of the mRNAs of the nicotinic sub-units in the aphid A. pisum at 24 h post-absorption of the 2 or LacZ dsRNAs.

FIG. 5C shows a histogram quantifying the expression of the mRNAs of the nicotinic sub-units in the aphid A. pisum at 72 h post-absorption of the 2 or LacZ dsRNAs.

FIG. 6 shows a graphical illustration of qPCR quantification of the expression of the transcripts of the nicotinic 31 sub-unit in the terminal abdominal ganglion (TAG) of cockroaches P. americana having ingested the dsRNAs (Lacz, β1-start, β1-end and mixture β1-start+end). Relative expression is normalized to the actin gene (reference gene) according to the 2-ΔΔCt method (*p<0.05 vs LacZ, Mann-Whitney test, n=4 to 8).

FIG. 7A shows a histogram depicting the corrected mortality rate 96 h after acute intoxication with imidacloprid (IMI in exposed cockroaches D30) and non-exposed (NE) cockroaches at the sublethal dose of imidacloprid for 30 days.

FIG. 7B shows a histogram depicting the corrected mortality rate 96 h after acute intoxication with imidacloprid in cockroaches exposed to the sublethal dose of imidacloprid for 30 days, which had ingested the dsRNAs (Lacz, β1-end or β1-start+end) or not (IMI D30). (n=1 including 8 to 9 cockroaches per condition).

FIG. 8A shows a graphical representation of qPCR quantification of the expression of the mRNAs of the nicotinic β2 sub-unit in aphid A. pisum larvae at 2 h, 24 h, 48 h and 72 h after contacting thereof with a nutrient solution containing the dsRNAs (dsRNA-LacZ or dsRNA-(2) at 200 ng/μL. Relative expression is normalized to the RPL7 gene (reference gene) according to the 2-ΔΔCt method (*p<0.05, dsRNA-β2 vs dsRNA-LacZ, non-parametric Mann-Whitney test; n=5 to 6 with 10 larvae per n).

FIG. 8B shows a histogram depicting the corrected mortality rate relative to the control (dsRNA-LacZ) obtained using the formula of Henderson-Tilton in aphid A. pisum larvae which had ingested dsRNA-β2 or dsRNA-LacZ (*p<0.05, **p<0.01; dsRNA-β2 vs dsRNA-LacZ, non-parametric Mann-Whitney test; n=7 to 8 with 20 larvae per n).

FIG. 9 shows a graphical representation of the qPCR quantification of the expression of the transcripts of the isoforms of the auxiliary proteins RIC-3, Lynx, NACHO and UNC-50 in larvae (A) and adult aphids (B) of A. pisum. Relative expression relative is normalized to the RPL7 gene (reference gene) according to the 2-ΔΔCt method (n=6).

### DETAILED DESCRIPTION OF THE INVENTION

The present invention relates to a method for controlling insect pests by inhibiting translation of the mRNAs of a target gene belonging to the cholinergic system of the insect, induced by RNA interference.

By “insect pest”, it is meant herein any insect with activity having effects considered to be harmful to public health and/or to the proper conducting of some human activities such as agriculture or breeding. Insect pests group together phytophagous, saprophagous and detritivorous insects, predator, parasitic, commensal, hematophagous insects.

Among phytophagous insects, particular mention can be made of Helicoverpa armigera, Bemisia tabaci, Plutella xylostella, Tribolium castaneum, Myzus persicae, Spodoptera frugiperda, Aphis gossypii, Nilaparvata lugens, Spodoptera exigua, Ceratitis capitata, Cydia pomonella, Acyrthosiphon pisum, Diaphorina citri or Thrips tabaci.

The expression “cholinergic system” includes the cholinergic receptors capable of binding acetylcholine, and the ligands thereof. Here the focus is solely on the nicotinic receptors, and not the muscarinic receptors.

By “RNA interference”, it is meant herein the technique allowing specific regulation of the expression of a protein by inhibiting translation of mRNA. The mechanism already mentioned in the foregoing will be detailed below.

In one particular embodiment, the method may comprise the steps of:
preparing a double-stranded RNA specific to a target mRNA;
administering the double-stranded RNA to at least one insect pest in an effective amount to induce mortality or sensitivity to an insecticide of the targeted insect.

In one particular alternative embodiment, the method may comprise the steps of:
preparing a single-stranded antisense oligonucleotide (ASO) specific to a target mRNA;
administering the single-stranded antisense oligonucleotide to at least one insect pest in an effective amount to induce mortality or sensitivity to an insecticide of the targeted insect.

Antisense oligonucleotides are small single-stranded nucleic acids (RNA or DNA) which are able to associate to form heteroduplexes with the target mRNA, inhibiting the function of the latter by impairing translation of the mRNA into a protein, or by causing degradation of the mRNA by recruiting RNAse H which hydrolyses the RNA in the RNA/DNA duplex. Their functioning differs according to the type of oligonucleotide used for gene silencing.

In one particular embodiment, the target mRNA can be chosen from the group comprising the mRNAs of the group of neuronal α sub-units of the nicotinic receptor, the mRNAs of the group of neuronal β sub-units of the nicotinic receptor, the mRNAs encoding the auxiliary proteins and molecules, and the isoforms thereof, and a protein of the nicotinic receptor interactome.

Known auxiliary proteins and molecules of the nicotinic receptor are for example NACHO, Lynx, TMX-3, RIC-3, UNC-50 and their isoforms.

NACHO (novel nAChR regulator) and RIC-3 (resistance to inhibitor of cholinesterase 3) are transmembrane chaperone proteins responsible for the folding and assembly of nAChRs in the endoplasmic reticulum. RIC-3 also appears to be necessary for directing nAChRs to the plasma membrane.

Lynx (Ly-6/neurotoxin protein) is an extracellular protein anchored to the plasma membrane via a glycosylphosphatidyl-inositol (GPI), which reduces the sensitivity of nAChR to acetylcholine.

UNC-50 (inner nuclear membrane RNA binding protein) is a protein located in the Golgi apparatus which allows regulation of nAChR biosynthesis and localization thereof to the plasma membrane.

By “interactome”, it is meant herein all the molecular interactions occurring with the nicotinic receptor within a cell, a tissue or organism, throughout the various physiological processes.

By “effective amount” for inducing mortality or sensitivity to an insecticide of a targeted insect, it is meant herein an amount allowing an increase in mortality of at least 10% compared with non-treatment, or an improvement in sensitivity of at least 10% compared with treatment by the insecticide alone. This effective amount is dependent on the efficacy of each double-stranded RNA or antisense oligonucleotide, on the functioning time thereof, on the type of administration, and on the targeted insect.

In one particular embodiment, the target mRNA can be at least one from among the mRNAs of the neuronal sub-units α1 to α10 of the nicotinic receptor, and the mRNAs of the neuronal sub-units β1 to β10 of the nicotinic receptor.

In one exemplary embodiment, the target mRNA can be chosen from the group consisting of the mRNAs of the group of neuronal α sub-units of the nicotinic receptor of Acyrthosiphon pisum, namely the mRNAs encoding the neuronal α1 sub-unit of the nicotinic receptor ID (SEQ NO: 1, accession n°: XM_008182407.3), the mRNAs encoding the neuronal α2 sub-unit of the nicotinic receptor (SEQ ID NO: 2, accession n°: XM_008182417.3), the mRNAs encoding the neuronal α3 sub-unit of the nicotinic receptor (SEQ ID NO: 3, accession n°: XM_008187942.3), the mRNAs encoding the isoforms of the neuronal α4 sub-unit of the nicotinic receptor (SEQ ID NO: 4, accession n°: XM_029490651.1; SEQ ID NO: 5, accession n°: XM_029490650.1), the mRNAs encoding the isoforms of the neuronal α6 sub-unit of the nicotinic receptor (SEQ ID NO: 6, accession n°: XM_016809607.2; SEQ ID NO: 7, accession n°: XM_016809606.2), the mRNAs encoding the isoforms of the neuronal α7 sub-unit of the nicotinic receptor (SEQ ID NO: 8, accession n°: XM_001945189.5; SEQ ID NO: 9, accession n°: XM_029490468.1; SEQ ID NO: 10, accession n°: XM_008187756.3; SEQ ID NO: 11, accession n°: XM_016806826.2; SEQ ID NO: 12, accession n°: XM_029490467.1; SEQ ID NO: 13, accession n°: XM_016806827.2), the mRNAs encoding the neuronal α8 sub-unit of the nicotinic receptor (SEQ ID NO: 14, accession n°: XM_001949983.5), the mRNAs encoding the neuronal α9 sub-unit of the nicotinic receptor (SEQ ID NO: 15, accession n°: XM_016807628.1), the mRNAs encoding the neuronal α10 sub-unit of the nicotinic receptor (SEQ ID 16, accession n°: XM_016807463.2), and the isoforms thereof.

In another examplary embodiment, the target mRNA can be chosen from the group consisting of the mRNAs of the group of neuronal β sub-units of the nicotinic receptor of Acyrthosiphon pisum, namely the mRNAs encoding the neuronal 31 sub-unit of the nicotinic receptor (SEQ ID NO: 17, accession n°: XM_029487953.1), and the mRNAs encoding the neuronal β2 sub-unit of the nicotinic receptor (SEQ ID NO: 18, accession n°: XM_001945029.5), and the isoforms thereof.

In a further examplary embodiment, the target mRNA can be chosen from the group consisting of the mRNAs of the isoforms of the auxiliary RIC-3 proteins (RIC-3 A, SEQ ID NO: 70, accession n°: XM_008186459.3; RIC-3 B, SEQ ID NO: 71, accession n°: XM_008186458.3, RIC-3 C, SEQ ID NO: 72 accession n°: XM_008186457.3), Lynx (Lynx 1, SEQ ID NO: 73, accession n°: NM_001162709.1; Lynx 4, SEQ ID NO: 74, accession n°: XM_001945238.5; Lynx 5, SEQ ID NO: 75, accession n°: XM_001950961.5; Lynx 6, SEQ ID NO: 76, accession n°: NM_001162627.2; Lynx 10C, SEQ ID NO: 77, accession n°: NM_001162777.2), NACHO (SEQ ID NO: 78, accession n°: NM_001205021.1), UNC-50 (SEQ ID NO: 79, accession n°: NM_001162643.2) and TMX-3 (SEQ ID NO: 80, accession n°: XM_008188638.3) of Acyrthosiphon pisum.

Preferably, the target mRNA can be chosen from among the mRNAs of the auxiliary proteins Lynx6 and NACHO of Acyrthosiphon pisum.

The protein sequences of these isoforms of the auxiliary proteins of Acyrthosiphon pisum are the following: RIC-3 (RIC-3 A, SEQ ID NO: 81, accession n°: XP_008184681.1; RIC-3 B, SEQ ID NO: 82, accession n°: XP_008184680.1, RIC-3 C, SEQ ID NO: 83, accession n°: XP_008184679.1), Lynx (Lynx 1, SEQ ID NO: 84, accession n°: NP_001156181.1; Lynx 4, SEQ ID NO: 85, accession n°: XP_001945273.1; Lynx 5, SEQ ID NO: 86, accession n°: XP_001950996.2; Lynx 6, SEQ ID NO: 87, accession n°: NP_001156099.1; Lynx 10C, SEQ ID NO: 88, accession n°: NP_001156249.1), NACHO (SEQ ID NO: 89, accession n°: NP_001191950.1), UNC-50 (SEQ ID NO: 90, accession n°: NP_001156115.1) and TMX-3 (SEQ ID NO: 91, accession n°: XP_008186860.1).

In one examplary embodiment, the target mRNA can be chosen from the group consisting of the mRNAs of the group of neuronal α sub-units of the nicotinic receptor of Periplaneta americana, namely the mRNAs encoding the neuronal α1 sub-unit of the nicotinic receptor (SEQ ID NO: 19, accession n°: KP725463.1), the mRNAs encoding the neuronal α2 sub-unit of the nicotinic receptor (SEQ ID NO: 20, accession n°: KP725464.1), the mRNAs encoding the neuronal α3 sub-unit of the nicotinic receptor (SEQ ID NO: 21, accession n°: KR021292.1), the mRNAs encoding the isoforms of the neuronal α4 sub-unit of the nicotinic receptor (SEQ ID NO: 22, accession n°: JN390946.1; SEQ ID NO: 23, accession n°: JN390945.1), the mRNAs coding for the neuronal α5 sub-unit of the nicotinic receptor (SEQ ID NO: 24, accession n°: GFCQ01005211.1), the mRNAs encoding the isoforms of the neuronal α6 sub-unit of the nicotinic receptor (SEQ ID NO: 25, accession n°: JF731243.1; SEQ ID NO: 26, accession n°: JX466887.1; SEQ ID NO: 27, accession n°: JX466888.1; SEQ ID NO: 28, accession n°: JX466889.1; SEQ ID NO: 29, accession n°: JX466890.1), the mRNAs encoding the isoforms of the neuronal α7 sub-unit of the nicotinic receptor (SEQ ID NO: 30, accession n°: MW201211.1; SEQ ID NO: 31, accession n°: JF731242.1; SEQ ID NO: 32, accession n°: MK790056.1; SEQ ID NO: 33, accession n°: JX466891.1), the mRNAs encoding the neuronal α8 sub-unit of the nicotinic receptor (SEQ ID NO: 34, accession n°: MW201212.1), the mRNAs encoding the neuronal α9 sub-unit of the nicotinic receptor (SEQ ID NO: 35, accession n°: MW201214.1), and the isoforms thereof.

In another examplary embodiment, the target mRNA can be chosen from the group consisting of the mRNAs of the group of neuronal β sub-units of the nicotinic receptor of Periplaneta americana, namely the mRNAs encoding the neuronal β1 sub-unit of the nicotinic receptor (SEQ ID NO: 36, accession n°: MW201213.1), the mRNAs encoding the neuronal β2 sub-unit of the nicotinic receptor (SEQ ID NO: 37, accession n°: GFCQ01032711.1), the mRNAs encoding the neuronal β3 sub-unit of the nicotinic receptor (SEQ ID NO: 38, accession n°: GFCQ01027461.1), the mRNAs encoding the neuronal β4 sub-unit of the nicotinic receptor (SEQ ID NO: 39, accession n°: GAWS02039241.1), the mRNAs encoding the neuronal β5 sub-unit of the nicotinic receptor (SEQ ID NO: 40, accession n°: GFCQ01009686.1), the mRNAs encoding the neuronal β6 sub-unit of the nicotinic receptor (SEQ ID NO: 41, accession n°: GFCQ01010089.1), the mRNAs encoding the neuronal β7 sub-unit of the nicotinic receptor (SEQ ID NO: 42, accession n°: GFCQ01012153.1), the mRNAs encoding the neuronal β8 sub-unit of the nicotinic receptor (SEQ ID NO: 43, accession n°: GFCQ01034959.1), the mRNAs encoding the neuronal β9 sub-unit of the nicotinic receptor (SEQ ID NO: 44, accession n°: GBJC01015771.1), the mRNAs encoding the neuronal (10 sub-unit of the nicotinic receptor (SEQ ID NO: 45, accession n°: GFCQ01027794.1), and the isoforms thereof.

For this purpose, the double-stranded RNA or single-stranded antisense oligonucleotide can be administered via a topical route, by spraying, vaporization, via nanoparticles like lipid nanoparticles, chitosan, liposomes, niosomes, cationic dendrimers, lipoplexes, via feeding, via trapping in a bait box, via crop irrigation.

The administering mode is not limited to the modes provided above, but it is within the reach of persons skilled in the art to include therein any other administration mode that is possible from an agricultural, agri-food, health and/or environmental perspective.

In one embodiment, the insect pest can be at least one chosen from phytophagous insects, saprophagous and detritivorous insects, predator insects, parasitic insects and commensal insects, hematophagous insects, and in particular from phytophagous insects and more particularly Helicoverpa armigera, Bemisia tabaci, Plutella xylostella, Tribolium castaneum, Myzus persicae, Spodoptera frugiperda, Aphis gossypii, Nilaparvata lugens, Spodoptera exigua, Ceratitis capitata, Cydia pomonella, Acyrthosiphon pisum, Diaphorina citri, Periplaneta americana or Thrips tabaci.

In particular, the insect pest is Acyrthosiphon pisum.

Aphids are currently one of the most crop-destructive insect pests. There exist about 5000 species of aphids worldwide of which 100 are pests having a significant economic impact through the destroying of crops. The damage caused by these insects is due first to the high number of offspring per aphid which is able to reproduce parthenogenetically, and secondly to the fact that these insects have piercing-sucking mouthparts feeding on the sap of plants and causing possible delayed growth, plant discoloring or deformation, and which can also transmit viruses. More specifically, the pea aphid A. pisum colonizes numerous s leguminous crops which represent about 13% of cultivated surface areas over the world. This aphid is responsible for hundreds of million dollar losses every year.

In another particular embodiment, the insect pest is Periplaneta americana.

In one particular embodiment of the invention, the double-stranded RNA or antisense oligonucleotide can be administered by feeding at least one insect with a transgenic organism expressing the double-stranded RNA or antisense oligonucleotide.

For this purpose, the transgenic organism can be a transgenic plant.

Examples of transgenic plants include legumes such as peas, beans, chickpeas, lentils, broad beans, field beans, soybean, alfalfa, lupin, sainfoin, trefoil, clover or vetch, cereals such as wheat, maize, sorghum, rye, barley, oats or rice, but also fruit and vegetable plants.

The invention also relates to an insecticide composition for insect pests, the composition comprising a double-stranded RNA or antisense oligonucleotide and at least one from among a transfection agent and a solvent, wherein said double-stranded RNA or antisense oligonucleotide comprises a nucleotide sequence having at least 90% identity with at least part of the sequence of a target mRNA, the target mRNA being chosen from the group comprising the coding sequences of the genes of the group of neuronal α sub-units of the nicotinic receptor, of the genes of the group of neuronal β sub-units of the nicotinic receptor, and the isoforms thereof, the nucleotide sequences of the genes encoding the auxiliary proteins and molecules of the nicotinic receptor, and the isoforms thereof, or encoding a protein of the nicotinic receptor interactome.

The composition of the invention is intended to be administered via a topical administration mode, via spraying, vaporization, via nanoparticles like lipid nanoparticles, chitosan, liposomes, niosomes, cationic dendrimers, lipoplexes, via feeding, via trapping in a bait box, via crop irrigation.

Administration is not limited to the modes provided above, but it is within the reach of persons skilled in the art to include therein any other administration mode that is possible from an agricultural, agri-food, health and/or environmental perspective.

In one examplary embodiment, the invention relates to an insecticide composition comprising a double-stranded RNA or antisense oligonucleotide and a transfection agent or solvent, wherein said double-stranded RNA or antisense oligonucleotide comprises a nucleotide sequence having at least 90% identity with at least part of the sequence of the target mRNA, the target mRNA being chosen from the group consisting of the coding sequences of the genes of the group of neuronal α sub-units of the nicotinic receptor of Acyrthosiphon pisum, namely the mRNAs encoding the neuronal α1 sub-unit of the nicotinic receptor (SEQ ID NO: 1), the mRNAs encoding the neuronal α2 sub-unit of the nicotinic receptor (SEQ ID NO: 2), the mRNAs encoding the neuronal α3 sub-unit of the nicotinic receptor (SEQ ID NO: 3), the mRNAs encoding the isoforms of the neuronal α4 sub-unit of the nicotinic receptor (SEQ ID NO: 4; SEQ ID NO: 5), the mRNAs encoding the isoforms of the neuronal α6 sub-unit of the nicotinic receptor (SEQ ID NO: 6; SEQ ID NO: 7), the mRNAs encoding the isoforms of the neuronal α7 sub-unit of the nicotinic receptor (SEQ ID NO: 8; SEQ ID NO: 9; SEQ ID NO: 10; SEQ ID NO: 11; SEQ ID NO: 12; SEQ ID NO: 13), the mRNAs encoding the neuronal α8 sub-unit of the nicotinic receptor (SEQ ID NO: 14), the mRNAs encoding the neuronal α9 sub-unit of the nicotinic receptor (SEQ ID NO: 15), the mRNAs encoding the neuronal α10 sub-unit of the nicotinic receptor (SEQ ID NO: 16), and the isoforms thereof, and from the group consisting of the coding sequences of the genes of the group of neuronal β sub-units of the nicotinic receptor of Acyrthosiphon pisum, namely the mRNAs encoding the neuronal β1 sub-unit of the nicotinic receptor (SEQ ID NO: 17) and the mRNAs encoding the neuronal β2 sub-unit of the nicotinic receptor (SEQ ID NO: 18), and the isoforms thereof.

In a further examplary embodiment, the target mRNA can be chosen from the group consisting of the mRNAs of the isoforms of the auxiliary RIC-3 proteins (RIC-3 A, SEQ ID NO: 70, accession n°: XM_008186459.3; RIC-3 B, SEQ ID NO: 71 accession n°: XM_008186458.3, RIC-3 C, SEQ ID NO: 72, accession n°: XM_008186457.3), Lynx (Lynx 1, SEQ ID NO: 73, accession n°: NM_001162709.1; Lynx 4, SEQ ID NO: 74, accession n°: XM_001945238.5; Lynx 5, SEQ ID NO: 75, accession n°: XM_001950961.5; Lynx 6, SEQ ID NO: 76, accession n°: NM_001162627.2; Lynx 10C, SEQ ID NO: 77, accession n°: NM_001162777.2), NACHO (SEQ ID NO: 78, accession n°: NM_001205021.1), UNC-50 (SEQ ID NO: 79, accession n°: NM_001162643.2) and TMX-3 (SEQ ID NO: 80, accession n°: XM_008188638.3) of Acyrthosiphon pisum.

Preferably, the target mRNA can be chosen from among the mRNAs of the auxiliary proteins Lynx6 and NACHO of Acyrthosiphon pisum.

In one particular embodiment, the agent promoting transfection may comprise, without being limited thereto, a lipid compound, a liposome, a niosome, a lipid nanoparticle, a dendrimer, an insect virus.

It is understood that it is within the reach of persons skilled in the art to adapt the transfection mode according to context and need. The techniques of RNA transfection are widely described in the scientific literature.

In one particular embodiment, the composition may additionally comprise one or more agents chosen from a synergizing agent, a repellent agent and an attractant agent.

The synergic (or synergizing) agent can be piperonyl butoxide (PBO) for example which inhibits detoxification enzymes. It acts by slowing the degradation of chemical toxic products in insects, and this mechanism enables the pesticide to be maintained in its toxic form for longer periods of time.

By “repellent agent” it is meant a natural or synthetic chemical substance used to repel the insect pest of interest, in particular to protect agricultural crops or harvests.

By “attractant agent” it is meant a natural or synthetic chemical substance used to attract the insect pest of interest, in particular to bait the insect or to lure it towards a trap.

In one particular embodiment, the composition may additionally comprise a carrier that is acceptable from an agricultural, agri-food, health and/or environmental perspective.

Said carrier refers to a non-toxic material which does not interfere with the efficacy of the biological action of the composition, and which is compatible with a biological system such as a cell, a cell culture, a tissue, or an organism.

In one particular embodiment, the composition is formulated in the form of a bait for the insect pest(s).

For this purpose, the composition may comprise an attractant intended to lure the insect towards the bait box so that it is trapped therein.

The invention also relates to a transgenic plant cell, plant tissue or plant comprising at least one nucleic acid which is transcribed to produce a double-stranded RNA, wherein the double-stranded RNA comprises a nucleotide sequence having at least 90% identity with at least part of the sequence of a target mRNA, the target mRNA being chosen from the group consisting of the coding sequences of the genes of the group of neuronal α sub-units of the nicotinic receptor, the coding sequences of the genes of the group of neuronal β sub-units of the nicotinic receptor, and the isoforms thereof, the nucleotide sequences of the genes encoding the auxiliary proteins and molecules of the nicotinic receptor, and the isoforms thereof, or encoding a protein of the nicotinic receptor interactome.

In one examplary embodiment, the invention relates to a plant cell, plant tissue or transgenic plant comprising one or more nucleic acids which are transcribed to produce a double-stranded RNA, wherein said double-stranded RNA comprises a nucleotide sequence which has at least 90% identity with at least part of the sequence of the target mRNA, the target mRNA being chosen from the group consisting of the coding sequences of the genes of the group of neuronal α sub-units of the nicotinic receptor of Acyrthosiphon pisum, namely the mRNAs encoding the neuronal α1 sub-unit of the nicotinic receptor (SEQ ID NO: 1), the mRNAs encoding the neuronal β2 sub-unit of the nicotinic receptor (SEQ ID NO: 2), the mRNAs encoding the neuronal α3 sub-unit of the nicotinic receptor (SEQ ID NO: 3), the mRNAs encoding the isoforms of the neuronal α4 sub-unit of the nicotinic receptor (SEQ ID NO: 4; SEQ ID NO: 5), the mRNAs encoding the isoforms of the neuronal α6 sub-unit of the nicotinic receptor (SEQ ID NO: 6; SEQ ID NO: 7), the mRNAs encoding the isoforms of the neuronal α7 sub-unit of the nicotinic receptor (SEQ ID NO: 8; SEQ ID NO: 9; SEQ ID NO: 10; SEQ ID NO: 11; SEQ ID NO: 12; SEQ ID NO: 13), the mRNAs encoding the neuronal α8 sub-unit of the nicotinic receptor (SEQ ID NO: 14), the mRNAs encoding the neuronal α9 sub-unit of the nicotinic receptor (SEQ ID NO: 15), the mRNAs encoding the neuronal α10 sub-unit of the nicotinic receptor (SEQ ID NO: 16), and the isoforms thereof, and from the group consisting of the coding sequences of the genes of the group of neuronal β sub-units of the nicotinic receptor of Acyrthosiphon pisum, namely the mRNAs encoding the neuronal β1 sub-unit of the nicotinic receptor (SEQ ID NO: 17) and the mRNAs encoding the neuronal β2 sub-unit of the nicotinic receptor (SEQ ID NO: 18), and the isoforms thereof.

In a still further examplary embodiment, the target mRNA can be chosen from the group consisting of the mRNAs of the isoforms of the auxiliary RIC-3 proteins (RIC-3 A, SEQ ID NO: 70, accession n°: XM_008186459.3; RIC-3 B, SEQ ID NO: 71, accession n°: XM_008186458.3, RIC-3 C, SEQ ID NO: 72, accession n°: XM_008186457.3), Lynx (Lynx 1, SEQ ID NO: 73, accession n°: NM_001162709.1; Lynx 4, SEQ ID NO: 74, accession n°: XM_001945238.5; Lynx 5, SEQ ID NO: 75, accession n°: XM_001950961.5; Lynx 6, SEQ ID NO: 76, accession n°: NM_001162627.2; Lynx 10C, SEQ ID NO: 77, accession n°: NM_001162777.2), NACHO (SEQ ID NO: 78, accession n°: NM_001205021.1), UNC-50 (SEQ ID NO: 79, accession: n° NM_001162643.2) and TMX-3 (SEQ ID NO: 80, accession n°: XM_008188638.3) of Acyrthosiphon pisum.

Preferably, the target mRNA can be chosen from the mRNAs of the auxiliary proteins Lynx6 and NACHO of Acyrthosiphon pisum.

For this purpose, the double-stranded RNA has a length of at least 20 base pairs, in particular a length of 20-2000 base pairs, more preferably a length of 20-900 base pairs.

The interfering RNA is able to inhibit translation of the mRNAs corresponding to the coding sequence of any one of the genes of the group of neuronal α sub-units of the nicotinic receptor, of the genes of the group of neuronal β sub-units of the nicotinic receptor, and the isoforms thereof, to any one of the nucleotide sequences of the genes encoding the auxiliary proteins and molecules of the nicotinic receptor, and the isoforms thereof, or to the DNA sequence encoding a protein of the nicotinic receptor interactome.

The antisense oligonucleotide is able to inhibit translation of the mRNAs corresponding to the coding sequence of any one of the genes of the group of neuronal α sub-units of the nicotinic receptor, of the genes of the group of neuronal β sub-units of the nicotinic receptor, and the isoforms thereof, to any one of the nucleotide sequences of the genes encoding the auxiliary proteins and molecules of the nicotinic receptor, and the isoforms thereof, or to the DNA sequence encoding a protein of the nicotinic receptor interactome.

In one examplary embodiment, the dsRNA or antisense oligonucleotide is able to inhibit translation of the mRNA corresponding to the coding sequence of any one of the genes of the group of neuronal α sub-units of the nicotinic receptor of Acyrthosiphon pisum, namely the mRNAs encoding the neuronal α1 sub-unit of the nicotinic receptor (SEQ ID NO: 1), the mRNAs encoding the neuronal α2 sub-unit of the nicotinic receptor (SEQ ID NO: 2), the mRNAs encoding the neuronal α3 sub-unit of the nicotinic receptor (SEQ ID NO: 3), the mRNAs encoding the isoforms of the neuronal α4 sub-unit of the nicotinic receptor (SEQ ID NO: 4; SEQ ID NO: 5), the mRNAs encoding the isoforms of the neuronal α6 sub-unit of the nicotinic receptor (SEQ ID NO: 6; SEQ ID NO: 7), the mRNAs encoding the isoforms of the neuronal α7 sub-unit of the nicotinic receptor (SEQ ID NO: 8; SEQ ID NO: 9; SEQ ID NO: 10; SEQ ID NO: 11; SEQ ID NO: 12; SEQ ID NO: 13), the mRNAs encoding the neuronal α8 sub-unit of the nicotinic receptor (SEQ ID NO: 14), the mRNAs encoding the neuronal α9 sub-unit of the nicotinic receptor (SEQ ID NO: 15), the mRNAs encoding the neuronal α10 sub-unit of the nicotinic receptor (SEQ ID NO: 16), and the isoforms thereof, and of any one of the genes of the group of neuronal β sub-units of the nicotinic receptor of Acyrthosiphon pisum, namely the mRNAS encoding the neuronal β1 sub-unit of the nicotinic receptor (SEQ ID NO: 17) and the mRNAs encoding the neuronal β2 sub-unit of the nicotinic receptor (SEQ ID NO: 18), and the isoforms thereof.

In a further examplary embodiment, the dsRNA or antisense oligonucleotide is able to inhibit translation of the mRNA corresponding to the coding sequence of any one of the genes of the group of isoforms of the auxiliary proteins RIC-3 (RIC-3 A, SEQ ID NO: 70, accession n°: XM_008186459.3; RIC-3 B, SEQ ID NO: 71, accession n°: XM_008186458.3, RIC-3 C, SEQ ID NO: 72, accession n°: XM_008186457.3), Lynx (Lynx 1, SEQ ID NO: 73, accession n°: NM_001162709.1; Lynx, SEQ ID NO: 74, accession n°: XM_001945238.5; Lynx 5, SEQ ID NO: 75, accession n°: XM_001950961.5; Lynx 6, SEQ ID NO: 76 accession n°: NM_001162627.2; Lynx 10C, SEQ ID NO: 77, accession n°: NM_001162777.2), NACHO (SEQ ID NO: 78, accession n°: NM_001205021.1), UNC-50 (SEQ ID NO: 79, accession n°: NM_001162643.2) and TMX-3 (SEQ ID NO: 80, accession n°: XM_008188638.3) of Acyrthosiphon pisum.

Preferably, the target mRNA can be chosen from the mRNAs of the auxiliary proteins Lynx6 and NACHO of Acyrthosiphon pisum.

The invention also relates to the use of interfering RNA, or antisense oligonucleotide, as bioinsecticide.

By “bioinsecticide” or “biological insecticide”, it is meant a form of insecticide based on microorganisms or natural products. These bioinsecticides are biological agents, or agents of biological origin, allowing the control of insect pests in a more eco-friendly manner.

In another embodiment, the invention relates to the use of interfering RNA or antisense oligonucleotide as an agent for synergizing the insecticidal effect of an insecticide, or of a molecule with insecticidal effect, against an insect pest.

By “agents synergizing the insecticidal effect” it is meant molecules allowing the effect of an insecticide to be increased, for example by increasing sensitivity thereto, while reducing the concentration required for its action.

Examples of synergizing agents in this context particularly comprise chemical molecules or microorganisms, such as insect viruses.

The expression “molecule having an insecticidal effect against an insect pest” is intended to describe complex natural substances such as essential oils or pheromones.

Examples of such substances particularly comprise aspic lavender essential oil, geranium essential oil, citronella essential oil, eucalyptus essential oil, peppermint essential oil, cryptomeria essential oil, thyme essential oil.

In addition, without being limited thereto, the insecticide may include neonicotinoids, in particular imidacloprid, clothianidin, acetamiprid, dinotefuran, nitenpyram, thiacloprid and thiamethoxam, spinosyns, butenolides, mesoionics, sulfoximines, carbamates, pyrethroids, oxadiazines and organophosphates, or the molecule with insecticidal effect, without being limited thereto, may include a natural substance, an essential oil, a pheromone.

In another embodiment, the invention relates to the use of interfering RNA or antisense oligonucleotide as an agent for restoring the sensitivity of an insect pest to an insecticide.

For this purpose, the interfering RNA or antisense oligonucleotide of interest helps restore an insect's sensitivity to an insecticide which may have been lost through too extensive and/or improper exposure to the insecticide. Numerous mechanisms both biochemical and physiological or even behavioural are developed by insects to evade the toxicity of insecticides.

This aspect is of interest and the experimental results obtained by the inventors indicate a particularly novel use of interfering RNAs or antisense oligonucleotides to overcome resistance to insecticides.

To better illustrate the subject of the present invention, a description is given below of nonlimiting, illustrative examples in connection with the appended drawings.

Referring to FIG. 1, it can be seen that the expression levels of the mRNAs encoding the nicotinic β2 sub-unit in the aphids A. pisum are significantly decreased by 51%, 43% and 41% in the aphids having absorbed β2 dsRNA, compared with the control aphids having absorbed LacZ dsRNA at 2 h, 24 h and 72 h, respectively. This helps check the efficacy of the dsRNAs on the expression of the transcripts of the nicotinic β2 sub-unit in the pea aphid.

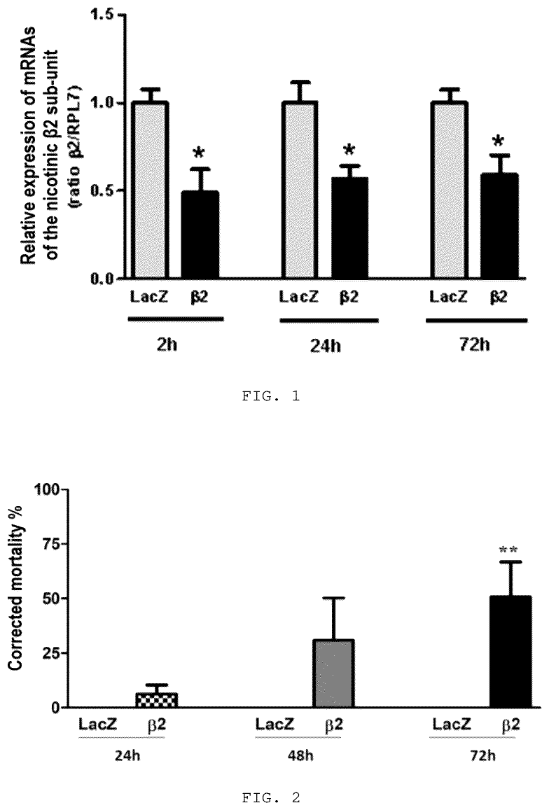

With reference to FIG. 2, it can be seen that the corrected mortality of the aphids at 24 h, 48 h and 72 h after topical application of the dsRNAs is increased in the aphids having absorbed the dsRNAs targeting the β2 sub-unit, compared with the control aphids (LacZ dsRNA) with an increase of about 6%, 31% and 51%, respectively.

With reference to FIG. 3, it can be seen that the curves of imidacloprid concentration/effect on the mortality of the aphids allow the LC₅₀ to be estimated at 8·10⁻³ μg/mL at 48 h and 4·10⁻³ μg/mL at 72 h. For the remainder of the experiments combining the dsRNAs directed against the nicotinic β2 sub-unit and imidacloprid, the imidacloprid concentration of 5·10⁻³ μg/mL was retained.

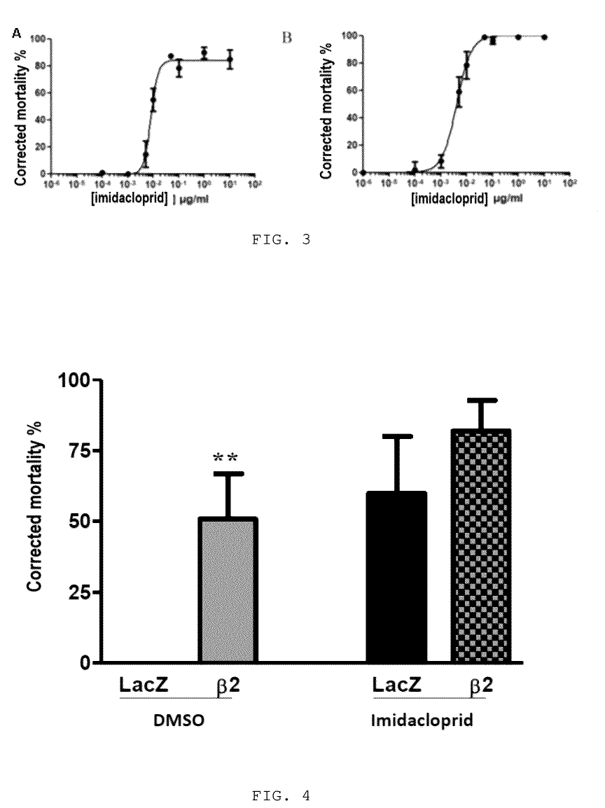

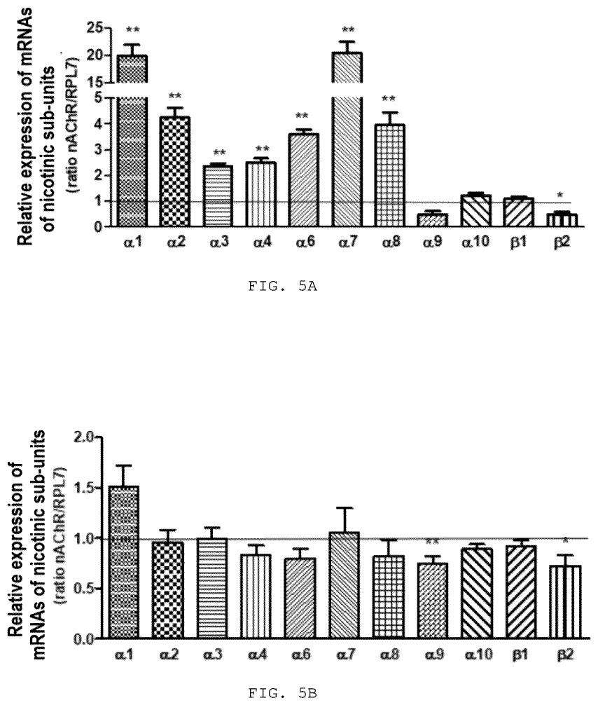

In FIG. 4, it can be seen that at 72 h post-intoxication with imidacloprid at 5·10⁻³ μg/mL, the percent mortality increases by about 1.4 times in the aphids having absorbed the dsRNAs targeting the β2 sub-unit (82%), compared with the control aphids (LacZ, 60%). DMSO, the imidacloprid solvent, was used as control.

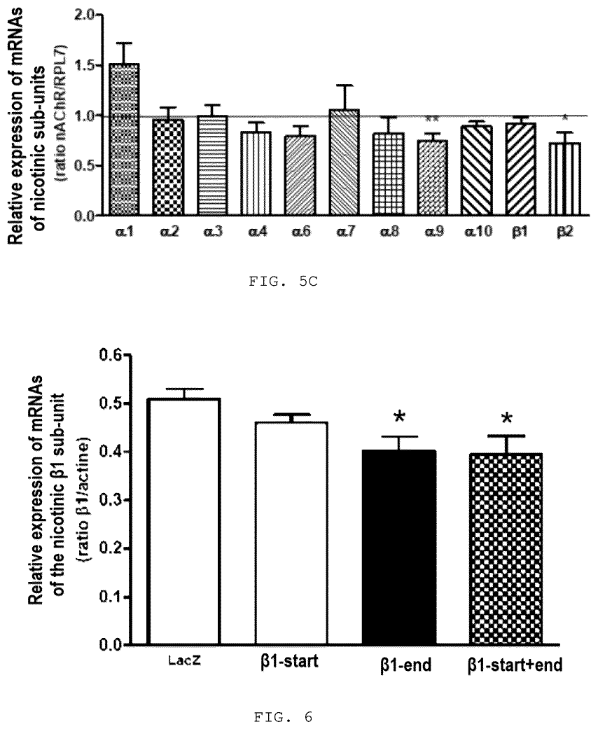

With reference to FIG. 5A, the expression levels of the mRNAs corresponding to the various nicotinic sub-units identified in the genome of the aphid A. pisum were quantified by qPCR from CDNAs derived from aphids having absorbed dsRNAs at different post-administration times. FIG. 5A shows that 2 hours after administering the dsRNAs, a significant increase in the expression of the mRNAs encoding the sub-units α1 to α8 is evidenced in the aphids which had absorbed the dsRNAs of interest, compared with the control aphids, whereas the expression of the sub-units α9, α10 and 1 is not modified. FIG. 5B shows that 24 h post-administration of the dsRNAs, solely the expression of the α9 sub-unit is affected by the decreased expression of the β2 sub-unit. FIG. 5C shows that at 72 h post-administration, the decrease in the expression of the β2 sub-unit does not appear to impact the expression of the other nicotinic sub-units.

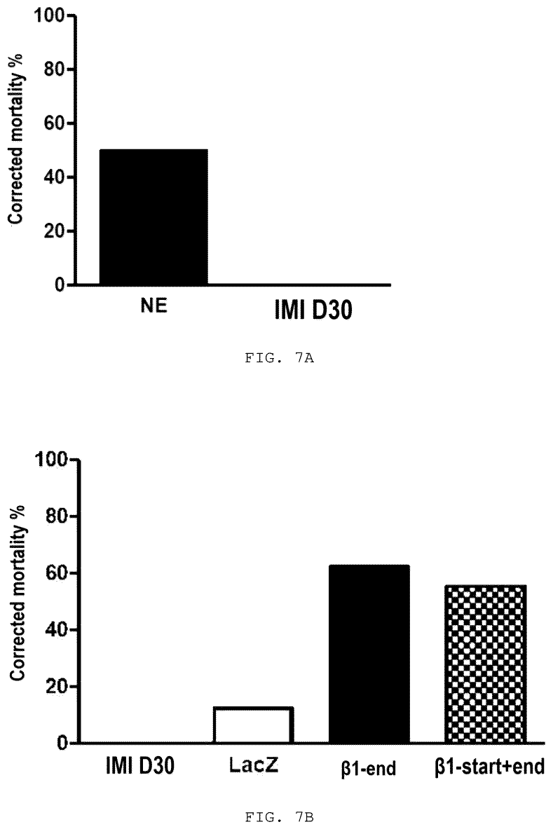

In FIG. 6, it can be noted that at 96 h post-ingestion of the dsRNAs, there is a decrease in the expression of the transcripts of the nicotinic β1 sub-unit of 20% in the TAGs derived from cockroaches exposed to β1-end dsRNA and to the mixture β1-start+end dsRNA. On the other hand, the expression of the mRNAs of the β1 sub-unit does not appear to be affected by B1-start dsRNA targeting the start of the nucleotide sequence of β1.

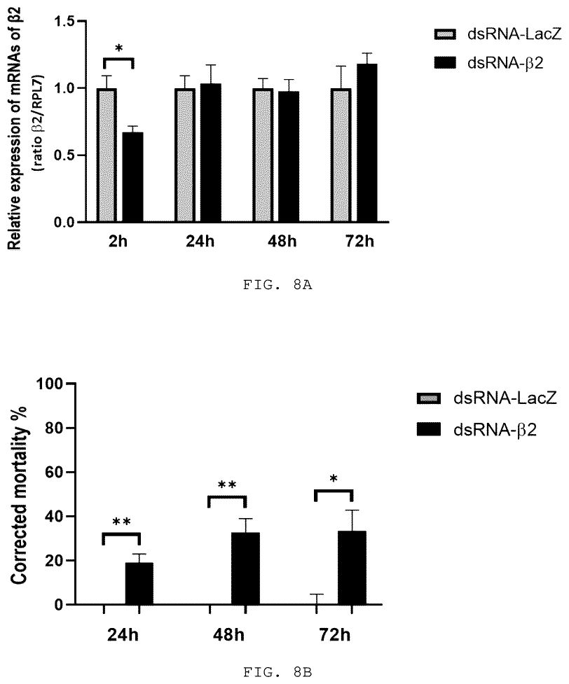

With reference to FIG. 7A, it can be seen that sublethal intoxication with imidacloprid for 30 days induces loss of sensitivity of the cockroaches to this insecticide after re-exposure of the cockroaches to an acute intoxication for 96 hours. FIG. 7B shows that, contrary to the cockroaches which had ingested LacZ dsRNA, corrected mortality rates of 55% and 60% were found 96 h after acute intoxication with imidacloprid in cockroaches which had ingested β1-end+start and β1-end dsRNA, respectively. These rates are similar to the rate obtained in cockroaches non-exposed to the sublethal dose of imidacloprid for 30 days, which seems to suggest that the dsRNAs, by modifying expression of the nAChRs, apparently allow bypassing of the resistance mechanisms set up by the insects, by restoring their sensitivity to the insecticide.

### EXAMPLES

The following examples illustrate the invention.

### Example 1: Development of the RNA Interference Technique Targeting the Nicotinic β2 Sub-Unit in the Pea Aphid A. Pisum as Means for Controlling Insect Pests

For this species of insect pest, the inventors focused on a divergent nicotinic sub-unit, the neuronal (2 sub-unit, to protect non-targeted organisms.

### Materials and Methods

Study Model: The Aphid Acyrthosiphon pisum

The pea aphids Acyrthosiphon pisum were bred in the SiFCIR laboratory (Laboratory for Functional Signalling of Ion Channels and Receptors-Angers, France) on field bean plants covered with a perforated cellophane bag sealed with an elastic band and placed in a chamber at a temperature of 20° C. with a photoperiod of 16 h light and 8 h darkness. To ensure this breeding, 7 larvae reproducing via parthenogenesis were placed on each field bean plant every 10 days. At Day 21, the adult aphids were used for the experiments.

### Extraction of Total RNAs

Extraction of total RNAS was realised using the NucleoSpin® RNA kit (Macherey-Nagel) on adult aphids. For this purpose, each aphid was ground in an Ultra-Turrax® in 350 μL of lysis buffer (RA1) was to which added 3.5 μL of β-mercaptoethanol, and the ground material was filtered. The nucleic acids contained in the lysate were precipitated with 70% ethanol (β50 μL) then bound onto a silica membrane. After DNase treatment and various washings of the membrane, the RNAs were eluted in 50 μL of H₂O. The quality and quantity of the RNAs obtained were evaluated by spectrophotometric assay (SimpliNano).

### Reverse Transcription

Synthesis of cDNA from the mRNAs was performed with the RevertAid H Minus First Strand CDNA Synthesis® kit (Thermoscientific). 500 ng of total RNAs (H₂O qs to 11 μL) were incubated at 65° C. for 5 minutes with 1 μL of oligo (dT) 18 primers (0.5 μg/μL) to enable hybridization of the primer at the poly (A) tail of the mRNAs. A reaction mixture of 8 μL composed of 4 μL of 5× reaction buffer, 1 μL of RiboLock RNase Inhibitor (20 U/μL), 2 μL of dNTP (10 mM) and 1 μL of the enzyme RevertAid H Minus M-MuLV Reverse Transcriptase (200 U/μL) was added. Reverse transcription was conducted for 1 h at 42° C. with a final step of 5 minutes at 70° C. to inactivate the reverse transcriptase. Finally, the cDNA samples obtained were stored at −20° C.

### PCR Amplification of the Sequences of Interest

To obtain the coding sequence of the β2 sub-unit (NCBI accession n°: XM_001945029.5—SEQ ID NO: 18) and the nucleotide fragment of 140 bp needed for synthesis of the dsRNAs targeting this sub-unit, amplification by PCR was performed on the cDNA of the aphid A. pisum with the high fidelity polymerase KOD Hot Start™ (Novagen) respectively using 32-ORF primers specific to the open reading frame, and 32-dsRNA primers comprising the sequence of the T7 promoter (5′-TAATACGACTCACTATAGGG-3′) (Table 1). Therefore, from a reaction mixture composed of 0.4 μL of DNA polymerase high fidelity KOD (1 U/μL, Novagen), 2 μL of KOD buffer (10×), 2 μL of dNTP (2 mM), 1.2 μL of MgSO₄ (10 mM), 1.2 μL of sense and antisense primers (10 μM), 2 μL of cDNA in 1:20 dilution and 1.2 μL of H₂O, the amplicons of interest were obtained after initial denaturation at 95° C. for 2 minutes followed by 30 cycles composed of 3 steps (denaturation for 20 seconds at 95° C., hybridization for 20 seconds at 60° C. and extension for 10 seconds at 70° C.) followed by final extension for 5 minutes at 72° C.

In parallel, amplification of part of the bacterial nucleotide sequence encoding β-galactosidase (140 bp), contained in the plasmid pCR-Blunt, was carried out using the specific LacZ-dsRNA primers (Table 1) associated with the sequence of the T7 promoter. This amplicon was used to produce control dsRNA.

TABLE 1
Table 1: Sequences of the primers used for cloning the β2
sub-unit, synthesis of dsRNAs and qPCR. The sequence of the T7
promoter is boxed.

| | | Size of | SEQ |
|---|---|---|---|
| | | amplicon | ID |
| | Primers (5′-3′) | (bp) | NO: |
| β2-ORF | Sense: AGCATTCTGACTACAGCGAG | 1519 | 46 |
| | Antisense: AACTAGTAGATAGATACACAAACATTGAT | | 47 |
| β2- | Sense: | 140 | |
| dsRNA | [symbol] TAGATGCTGCAATAACAGAC | | 48 |
| | Antisense: | | |
| | [symbol] AGTGTCTATCGTAGAACCTC | | 49 |
| LacZ- | Sense: | 140 | |
| dsRNA | [symbol] ATGACCATGATTACGCCAAG | | 50 |
| | Antisense: | | |
| | [symbol] TGGCGGCCGTTACTAGTGGA | | 51 |
| β2- | Sense: CGCAAAGACGAAGAGTCGAG | 151 | 52 |
| qPCR | Antisense: ACGGATAGCGTCAGGAACAC | | 53 |
| RPL7- | Sense: ACGTAAAGAGCGCGTGAAGA | 182 | 54 |
| qPCR | Antisense: GGTTCACACCACGAATACGCA | | 55 |

### Purification of PCR Products

After electrophoretic migration on 2% agarose gel, the PCR products were purified using the NucleoSpin® Gel and PCR clean-up kit (Macherey-Nagel). Briefly, the portion of agarose gel containing the amplicons of interest was dissolved over a hot water bath at 50° C. in NT1 buffer (200 μL per 100 mg of gel) containing chaotropic salts to allow binding of the DNA to a silica membrane. After complete dissolution of the gel, the amplicons were retained on a silica membrane, and two washings were performed with 700 μL of NT3 buffer (centrifugations of 30 seconds at 11000 g). The PCR products were eluted in 30 μL of H₂O by centrifugation of 1 minute at 11000 g. The quality and quantity of DNA were evaluated by spectrophotometry (SimpliNano).

### Molecular Cloning of the Nicotinic 2 Sub-Unit

Cloning of the β2 sub-unit was carried out with the Zero Blunt© PCR Cloning kit (Invitrogen). For cloning, the purified amplicons corresponding to the coding sequence of the β2 sub-unit were inserted in the PCR-Blunt cloning vector in an “insert: vector” ratio of 10:1. 1 μL of plasmid pCR-Blunt (25 ng/L), 6 μL of purified insert, 2 μL of ExpressLink T4 DNA ligase reaction buffer (5×), and 1 μL of ExpressLink T4 DNA ligase enzyme (5 U/μL) were incubated for 30 minutes at room temperature. Next, 4 μL of ligation product were inserted in One Shot© Top 10 chemically competent E. Coli bacteria after bacterial transformation via heat shock (30 minutes in ice, then 45 seconds at 42° C., and finally 2 minutes in ice). The transformed bacteria were incubated for 1 h at 37° C. in 250 μL of SOC medium, then spread on Petri dishes containing a selective LB-agar medium to which kanamycin was added (50 g/mL). After incubation overnight at 37° C., the recombinant clones were selected by PCR using M13 primers specific to the plasmid sequences flanking the insert, then placed in culture overnight in 5 mL of LB-kanamycin medium at 37° C. under stirring (225 rpm). The recombinant plasmids were finally purified with the NucleoSpin Plasmid® kit (Macherey-Nagel) and sequenced (Eurofins).

In Vitro Transcription and Synthesis of the dsRNAs

The purified PCR products (140 bp) specific to part of the coding sequence of the β2 sub-unit, or to the β-galactosidase containing the sequence of the T7 promoter upstream and downstream, were transcribed in vitro with the MEGAscript® T7 High Yield Transcription kit (Ambion). The transcription reaction was carried out at 37° C. overnight from a 20 μL reaction mixture composed of 100 ng of PCR products, 2 μL of each nucleotide (ATP, GTP, CTP, UTP; 75 mM), 2 μL of reaction buffer (10×), 2 μL of T7 RNA polymerase enzyme. Treatment with TURBO DNase (20) was then carried out for 15 minutes at 37° C. to remove the DNA matrix, and the transcription products were purified using the NucleoSpin® miRNA kit (Macherey-Nagel). First, 150 μL of ML buffer and 200 μL of absolute ethanol were added to the transcription products (qs to 150 μL H₂O) and the mixture was deposited on a silica membrane to allow binding of the DNA and large RNA fragments (>200 bp). After centrifuging at 11000 g for 30 seconds, 100 μL of MP buffer and 800 μL of MX buffer were added to the eluate containing the RNA fragments smaller than 200 bp such as the dsRNAs to allow precipitation and binding thereof to the silica membrane. In parallel, treatment with DNAse for 15 minutes at room temperature was performed on the silica membrane previously desalted with 350 μL of MDB buffer (centrifugation for one minute at 11000 g). After binding of the small RNA fragments to the silica membrane (centrifugation for 30 sec at 11000 g), the membrane was washed twice with MW2 buffer, the dsRNAs were eluted in 30 μL H₂O. Finally, the dsRNAs were denatured for 5 minutes at 95° C. then rehybridized for 1 h30 at room temperature. The quality and quantity of dsRNAs were evaluated by a spectrophotometer (SimpliNano). The dsRNAs obtained were stored at −20° C.

Preparation of the Lipoplexes Containing the dsRNAs and Topical Administration

To facilitate absorption of the dsRNAs by the aphids and to limit degradation of the dsRNAs, liposomes containing the various dsRNAs (lipoplexes) were prepared. To that end, 400 ng of dsRNA (β2 or LacZ) were contacted with 0.1 μL of Escort IV® transfection agent (Sigma-Aldrich) in the presence of PBS 1× (qs to 1 μL) for 30 minutes at room temperature. The dsRNAs were absorbed after topical administration to adult aphids according to Niu et al (2019). Briefly, the aphids were immobilised with a piece of Parafilm® that was previously pierced to provide access to the abdomen of the aphid. 1 μL of lipoplexes containing the dsRNAs was deposited on the aphid abdomen. A 30-minute waiting time is needed before the drop fully enters the aphid.

Quantitative PCR Analysis of the Expression of the β2 Sub-Unit in Aphids Having Absorbed the dsRNAs

Using the cDNAs derived from the various batches of aphids which had absorbed dsRNAs (β2 or LacZ), quantification of the transcripts of the β2 sub-units was performed by quantitative PCR (qPCR) to evaluate the effect of the dsRNAs targeting the β2 sub-unit on the expression of this sub-unit at different post-administration time points (2 h, 24 h and 72 h), and on the expression of the different nicotinic sub-units to evidence potential compensation phenomena. Each qPCR reaction required 5 μL of reaction buffer Takyon No Rox SYBR® Master Mix Blue dTTP (Eurogentec), 1 μL of sense and antisense primers (10 μM) (Table 1), 0.5 μL of H₂O and 2.5 μL of cDNA in 1:10 dilution. The quantification programme entailed a 1st step of 3 minutes at 95° C., followed by 40 denaturing cycles of 10 seconds at 95° C., and pairing and extension for 1 minute at the hybridization temperature of the primers (60° C.). Finally, an additional melting curve step was performed to allow gradual passing of the temperature from 50° C. to 95° C. to check the specificity of the primers. The efficacy of the various qPCR amplifications was between 97% and 102%. All the reactions were performed in duplicate and the values of threshold cycles (Ct) were normalized to the reference gene encoding the RPL7 protein. The intensity of fluorescence emitted by the SYBR® Green I probe was detected by the CFX Connect™ Real-Time PCR Detection System thermal cycler (Biorad), and the data were then analysed with CFX Maestro™ Software. The relative expression of the nicotinic sub-units was evaluated according to the 2^{-ΔΔCt} method.

### Mortality Tests

Mortality tests were performed using an artificial nutrition system comprising a PVC tube closed by a Parafilm® at each end, in which 5 adult aphids or 20 larvae were deposited. At one of the ends of the artificial nutrition “cell”, 200 μL of nutrient solution comprising the solution to be tested (insecticide, dsRNA) were deposited on the Parafilm then covered with a 2^{nd} Parafilm to allow the aphids to suck the solution for feeding by piercing the Parafilm with their rostrum. The cells were placed at 20° C. in a chamber with a photoperiod of 16 h. To determine the concentration of imidacloprid leading to 50% mortality (LC₅₀) in the aphid larvae, a concentration-effect curve was plotted at 48 h and 72 h from a range of imidacloprid concentrations from 10⁻⁶ to 10 μg/mL.

For the purpose of evaluating the efficacy of the strategy using interfering RNAs as bioinsecticide, the mortality of the adult aphids was observed at 24 h, 48 h and 72 h after absorption of the dsRNAs. The modulating effect of the dsRNAs on the sensitivity of the aphids to imidacloprid was studied by combining topical administration of dsRNA and toxicological tests with imidacloprid.

Acute intoxication with imidacloprid (5·10⁻³ μg/mL) was carried out 2 h after absorption of the dsRNAs, and the mortality of the aphids was assessed at 24 h, 48 h and 72 h.

Death of an insect was validated by absence of movement observed under a stereomicroscope. The results are expressed as percent corrected mortality using the formula of Henderson-Tilton to take into account the mortality of the aphids under the control conditions:

$$Corrected mortality = ( 1 - \frac{nv β 2 * ( nvLacZ + nmLacZ )}{nvLacZ * ( nv β2 + nm β2 )} ) * 100 \qquad [ formula 1 ]$$

where nᵥLacZ is the number of living aphids under the control condition; n_{M}Lacz is the number of dead aphids under the control condition; nᵥß2 is the number of living aphids having absorbed β2 dsRNA and n_{M}ß2 is the number of dead aphids having absorbed β2 dsRNA.

### Statistical Analyses

Statistical analyses of the qPCR results were performed with a Mann-Withney test, using GraphPad Prism software (version 8.0.1, GraphPad Software) where the p values lower than 0.05 are considered to be significant (*p<0.05, **p<0.01 and ***p<0.001).

The mortality curves were obtained with GraphPad Prism software in the form of a non-linear regression and adjusted with the Hill equation,

$$Y = m + ( {X}^{nH} ) \frac{( M - m )}{{X}^{nH} + {LC}_{50}^{nH}} \qquad [ formula 2 ]$$

where Y is the percent mortality observed at the concentration value X, m and M are respectively the minimum and maximum values of the curve, nH is the Hill coefficient and LC₅₀ is the concentration of imidacloprid causing 50% mortality. The results are expressed as a mean±S.E.M.
   Type of Effect of the dsRNAs

To determine the type of effect of the dsRNAs targeting the β2 sub-unit on the sensitivity of the aphids to imidacloprid, the results were analysed with the Model Deviation Ratio formula (MDR):

$$MDR = observed toxicity / expected toxicity$$

where the observed toxicity corresponds to percent corrected mortality of the aphids which had absorbed β2 dsRNA and had been exposed to imidacloprid, and expected toxicity corresponds to the sum of percent corrected mortalities obtained in the aphids of each condition taken individually (β2 dsRNA and imidacloprid). The MDR results are interpreted as follows: if the MDR is higher than or equal to 1.3, the effect is considered to be synergic; if the MDR is lower than or equal to 0.7, then the effect is antagonistic; and if the MDR is between 0.7 and 1.3, the effect is additive.

### Results

Preparation of the pCR-Blunt Plasmid Containing the Nucleotide Sequence Encoding the Nicotinic 2 Sub-Unit

To confirm the nucleotide sequence (accession n° XM_001945029.5, SEQ ID NO: 18) and protein sequence (accession M° XP_001945064.2) encoding the β2 sub-unit deposited in the NCBI database, in the aphids of the SiFCIR laboratory, the plasmid construct using the pCR-Blunt vector was prepared. The sequence inserted into the recombinant plasmid obtained after this cloning showed 100% homology with the deposited protein sequence.

Efficacy of the dsRNAs on the Expression of the Transcripts of the Nicotinic β2 Sub-Unit

For the purpose of determining the efficacy of the dsRNAs targeting the β2 sub-unit, the expression levels of the mRNAs encoding this protein were quantified by qPCR in adult aphids which had absorbed the dsRNAs (β2 or LacZ). This quantification was performed at various post-absorption time points (2 h, 24 h and 72 h). Significant decreases in the expression of the 2 sub-unit of 51%, 43% and 41% were observed in the aphids that had absorbed β2 dsRNA compared with the control aphids that had absorbed LacZ dsRNA at 2 h, 24 h and 72 h respectively (*p<0.05; FIG. 1). For the toxicological tests in the presence of imidacloprid associated with the absorption of the dsRNAs, the incubation time of the dsRNAs of 2 h was retained.

Effect of the dsRNAs on Aphid Mortality

For the purpose of verifying whether the dsRNAs targeting the β2 sub-unit are able to be used as bioinsecticide, the inventors evaluated the mortality of the aphids 24 h, 48 h and 72 h after topical application of the dsRNAs. Preliminary results suggest an increase in mortality in the aphids having absorbed the dsRNA targeting the β2 sub-unit (β2), compared with the control aphids (LacZ), of about 6%, 31% and 51% at 24 h, 48 h and 72 h, respectively (FIG. 2; n=5 including 15 to 30 aphids per condition). Therefore, the use of the dsRNAs as bioinsecticide can be envisaged under these experimental conditions, namely a single topical administration of 400 ng per aphid.

### Evaluation of the Sensitivity of the Aphids to Imidacloprid and Determination of the LC₅₀

Before examining the implication of the β2 sub-unit in the modulation of sensitivity of the aphids to imidacloprid, the concentration of imidacloprid causing 50% mortality of the insects was determined. For this purpose, various concentrations of imidacloprid ranging from 10⁻⁶ to 10 μg/mL were tested. From the concentration-effect curve, LC₅₀ was estimated at 8·10⁻³ μg/mL and 4·10⁻³ μg/mL at 48 h and 72 h respectively (FIG. 3). The concentration of 5·10⁻³ g/mL was retained for the experiments associating the dsRNAs targeting the β2 sub-unit and imidacloprid.

Effect of the dsRNAs on Aphid Sensitivity to Imidacloprid

Having regard to the fact that the composition of the nAChRs in nicotinic sub-units determines their sensitivity to insecticides, the decrease in the expression of the mRNAs of the β2 sub-unit by the specific dsRNAs observed in the aphids suggests a change in the composition of the nAChRs inducing a modulated efficacy of an insecticide treatment. To evaluate the effects of the dsRNAs as modulator of the efficacy of an insecticide treatment, toxicological tests in the presence of imidacloprid were carried out. For this purpose, adult aphids having absorbed the dsRNAs were intoxicated with imidacloprid at a concentration close to LC₅₀ namely 5·10⁻³ μg/mL. 72 h after acute intoxication, a slight increase in mortality (×1.4 times) was observed in the aphids having absorbed the dsRNAs directed against the β2 sub-unit, compared with the control aphids (LacZ) (FIG. 4). Therefore, the use of the specific dsRNAs inducing a modification of the composition of the nAChRs would render the insects more sensitive to the insecticide.

To determine the type of effect brought by the association “β2 dsRNA and imidacloprid”, the results were analysed with the Model Deviation Ratio (MDR) and the value of MDR was evaluated at 0.74. Therefore, with MDR being between 0.7 and 1.3, the effect of the association of dsRNA against the β2 sub-unit and imidacloprid is considered to be additive.

Effect of the Decrease of the β2 Sub-Unit on the Expression of the Other Nicotinic Sub-Units in the Aphid A. pisum

To correlate the modified sensitivity of the aphids having absorbed the dsRNA targeting 2 with possible changes in the composition of the nAChRs, the expression levels of the mRNAs corresponding to the various nicotinic sub-units identified in the genome were quantified by qPCR from CDNAs derived from the aphids having absorbed dsRNAs at various post-administration time points (2 h, 24 h and 72 h). Two hours after administration of the dsRNAs, a significant increase in the expression of the mRNAs encoding the α1 to α8 sub-units was evidenced in the aphids which had absorbed the dsRNAs of interest, compared with the control aphids (FIG. 5A) whereas the expression of the α9, α10 and β1 sub-units was not modified.

At 24 h post-administration of the dsRNAs, solely the decrease in the expression of the α9 sub-unit was associated with the decreased expression of the β2 sub-unit in aphids which had absorbed β2 dsRNA (FIG. 5B). Finally, the decrease in the expression of the β2 sub-unit induced by β2 dsRNA observed at 72 h does not appear to impact the expression of the other nicotinic sub-units (FIG. 5C).

These results make it possible to envisage interfering RNAs as potentializing agent of an insecticide such as imidacloprid, with a perspective to reduce the amount of insecticide used to control insect pests, complying with government guidelines.

The inventors have therefore been able to evidence an increase in the mortality of aphids, which had absorbed the dsRNAs targeting the β2 sub-unit, by about 40% from 48 h to 72 h post-absorption, compared with the control aphids. These results point towards the opportunity of using dsRNAs directed against the β2 sub-unit as bioinsecticide and suggest that this sub-unit is essential in the aphid.

Finally, the aphids having absorbed the dsRNAs targeting the β2 sub-unit exhibit better sensitivity to imidacloprid compared with the control aphids which had absorbed LacZ dsRNA. The results of the toxicological tests suggest that the decrease in the expression of the β2 sub-unit induced by the interfering RNAs of interest could lead to a change in the composition of the nicotinic receptors in aphids, rendering them more sensitive to the insecticide.

### Example 2: Use of the RNA Interference Technique Targeting a Nicotinic Sub-Unit in the Cockroach as Means for Restoring Sensitivity of the Insect Pest that had Become Resistant to the Insecticide

In this study, the cockroach P. americana was used as a model. This insect, which the genome has recently been sequenced but not annotated, is able to adapt to an insecticide treatment by modifying its composition of nicotinic sub-units (Benzidane Y et al., 2017) and has neuronal targets of insecticides that are well identified and characterised (Thany S H et al., 2007; Sinakevitch I G et al., 1996; Grolleau F et al., 2000).

To date, 19 nicotinic sub-units have been identified in P. americana and deposited in the NCBI database: 9 α sub-units (α1 to α9) and 10 β sub-units (B1 to β10) (Jones et al., 2021). Since the β1 sub-unit belongs to most heteropentameric nAChRs, it represents a target of choice for dsRNAs to develop the RNA interference technique in the context of the invention.

### Materials and Methods

Production of dsRNAs

### Identification of Fragments of Interest Specific to the Nicotinic β1 Sub-Unit

To determine the nucleotide fragments (100 bp) specific to the coding sequence of the 1 sub-unit (NCBI accession n°: MW201213.1—SEQ ID NO: 36) needed for synthesis of the dsRNAs, the alignment of the various nicotinic sub-units identified in the cockroach P. americana was performed with the Clustal Omega software.

### PCR Amplification of Specific Fragments

The pCR-Blunt plasmid in which the nucleotide sequence of the nicotinic β1 sub-unit was inserted (pCR-Blunt/β1) was used as matrix to obtain the fragments of interest. PCR amplification was performed with the high fidelity KOD Hot Start™ DNA polymerase (Novagen) using the specific dsRNA-β1 primers (Table 2) completed with the sequence of the T7 promoter (5′-TAATACGACTCACTATAGGG-3′) according to the following reaction mixture: 0.4 μL KOD DNA polymerase (1 U/μL), 2 μL of KOD buffer (10×), 2 μL of dNTP (2 mM), 1.2 μL of MgSO₄ (10 mM), 1.2 μL of sense and antisense primers (10 μM), 2 μL of plasmid pCR-Blunt/B1 (10 ng/μL) and H₂O qs to 20 μL.

The amplicons were obtained after initial denaturation at 95° C. for 2 minutes followed by 30 cycles composed of 3 steps (denaturation for 20 seconds at 95° C., hybridization for 20 seconds at 60° C. and extension for 10 seconds at 70° C.) followed by a final extension for 7 minutes at 70° C.

In parallel, amplification of part of the bacterial nucleotide sequence encoding the β-galactosidase (100 bp), contained in the pCR-Blunt plasmid, was carried out using the specific LacZ primers (Table 2) associated with the sequence of the T7 promoter. This amplicon was used to produce control dsRNA.

TABLE 2

| | | | Position | |
|---|---|---|---|---|
| | | | on | |
| | | | the | |
| | | Size of | sequence | SEQ |
| | | amplicon | (base | ID |
| Primers (5′-3′) | | (bp) | n°) | NO: |
| β1- | Sense: | 140 | 1-100 | 57 |
| start | 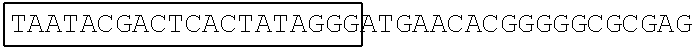 | | | |
| | C | | | |
| | Antisense: | | | 58 |
| | 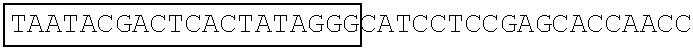 | | | |
| | A | | | |
| β1- | Sense: | 140 | 1123- | 59 |
| end | 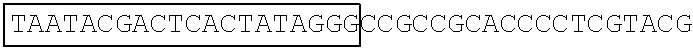 | | 1222 | |
| | G | | | |
| | Antisense: | | | 60 |
| | 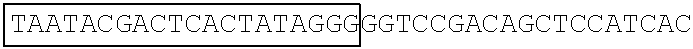 | | | |
| | C | | | |
| LacZ | Sense: | 140 | 1-100 | 61 |
| | 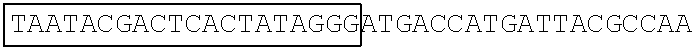 | | | |
| | G | | | |
| | Antisense: | | | 62 |
| | 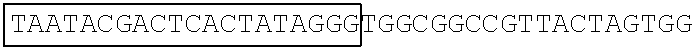 | | | |
| | A | | | |

Table 2: Sequences of the primers used for synthesis of the dsRNAs. The sequence of the T7 promoter is boxed.

Purification of the PCR Products, In Vitro Transcription, and dsRNA Synthesis

The protocol detailed in the foregoing for the pea aphid was used.

Study Model: Cockroaches Periplaneta americana

The P. americana cockroaches were bred in the SiFCIR laboratory in vivariums at a temperature of 29° C. and cyclic photoperiod of 12 h light and 12 h darkness. They were fed and hydrated ad libitum.

For the purpose of bypassing the resistance mechanisms set up by the insect, the dsRNAs were tested on male cockroaches having been exposed for 30 days to a sublethal dose of imidacloprid (0.025 μg/cockroach/day) according to Benzidane et al. (2017).

Preparation of the Lipoplexes Containing the dsRNAs

For oral administration of the dsRNAs to the cockroaches and for better stability of these dsRNAs, lipoplexes, liposomes containing the different dsRNAs (β1-start, β1-end or LacZ were formed. To that end, 0.25 μg of dsRNA (β1-start, β1-end, β1-start+β1-end, LacZ) at 0.1 μg/μL were contacted with 1 μL of transfection agent Escort IV® (Sigma-Aldrich) in the presence of 6.5 μL of 5% glucose solution for 30 minutes at room temperature.

Ingestion of the dsRNAs by the Cockroaches

The dsRNAs were administered to the cockroaches via oral route. In short, using a P10 pipette, 10 μL of lipoplex solution containing the dsRNAs (β1-start, β1-end, β1-start+end, LacZ) were ingested by the cockroaches immobilised by the wings. The effect of the dsRNAs as bioinsecticide was evaluated by observation of the mortality at 24 h, 48 h, 72 h and 96 h post-ingestion.

### Dissection of the Terminal Abdominal Ganglion and Extraction of Total RNAs

96 hours after ingestion of the dsRNAs, the cockroaches were dissected to extract the terminal abdominal ganglion (TAG) containing the DUM neurons. Extraction of total RNAs from the TAGs was performed with the Nucleospin® RNA kit (Macherey-Nagel). The quantity of extracted RNA was evaluated by UV spectrophotometry assay (SimpliNano).

### Reverse Transcription

Reverse transcription, allowing synthesis of cDNA from mRNA, was performed with the Revertaid H Minus First Strand CDNA Synthesis® kit (ThermoScientific) as described above.

Quantitative PCR Analysis of the Expression of the β1 Sub-Unit in Cockroaches Having Ingested the dsRNAs

From the cDNAs derived from the various batches of cockroaches which had ingested the dsRNAs (β1-start, β1-end, β1-start+end, LacZ), quantification of the transcripts of the β1 sub-unit was performed via quantitative PCR (qPCR) to evaluate the effect of the dsRNAs on the expression of the β1 sub-unit. The protocol was adapted to the one used for the pea aphid using as sense and antisense primers the primers given in Table 3 (2.5 μM for the β1 sub-unit and 10 μM for actin used as reference gene, and the hybridization temperature of the primers is also given in Table 3).

TABLE 3
Table 3: Sequences of the primers used for qPCR

| | | | | | SEQ |
|---|---|---|---|---|---|
| | Sense primers | Antisense primers | | Hybridization | ID |
| Gene | (5′-3′) | (5′-3′) | Accession No | Tº (º C.) | NO: |
| β1 | GGTGACCAAGTGTCCTTAG | ATGATTGCCCTCGTAGATG | MW201213.1 | 65 | 63; |
| | | | | | 64 |
| Actin | GACTACTGGTATTGTGCTGG | AAAGCTGTAACCACGCTCAG | AY116670.1 | 60 | 65; |
| | | | | | 66 |

### Results

### Identification of the Fragments of Interest Specific to the Nicotinic β1 Sub-Unit

Further to biocomputational analysis using alignment of the various nucleotide sequences of the nicotinic sub-units of the cockroach P. americana deposited in the NCBI database, 2 fragments of 100 bp specific to the nicotinic β1 sub-unit were identified. The first is positioned at the start of the nucleotide sequence (nucleotide n°1 to 100—SEQ ID NO: 67) while the second is positioned towards the end (nucleotide n°1123-1222—SEQ ID NO: 68).

Effect of the dsRNAs on the Expression of the Transcripts of the Nicotinic β1 Sub-Unit

The inventors were able to evidence, at 96 h post-ingestion, a decrease in the expression of the transcripts of the β1 sub-unit of 20% in the TAGs derived from the cockroaches exposed to dsRNA-β1-end and to the mixture dsRNA-β1-start and end (*p<0.05). On the other hand, the expression of the mRNAs of the β1 sub-unit does not appear to be affected by dsRNA β1-start targeting the start of the nucleotide sequence of β1 (FIG. 6).

For the remainder of the experiments, only dsRNA β1-end and the mixture dsRNA β1-start and β1-end were ingested by the cockroaches for the toxicological tests.

Use of the dsRNAs to Bypass the Resistance Mechanisms of the Cockroach P. americana

To study the efficacy of the dsRNAs in restoring the loss of sensitivity observed in resistant insects, toxicological tests were conducted using cockroaches rendered less sensitive to imidacloprid after exposure to a sublethal dose of imidacloprid. At a first stage, the inventors verified that sublethal intoxication with imidacloprid for 30 days induced loss of sensitivity of the cockroaches to imidacloprid (FIG. 7A).

Contrary to the cockroaches having ingested LacZ dsRNA, 55% and 60% mortality rates were observed 96 h after acute intoxication with imidacloprid in those cockroaches which had ingested β1-end+start and β1-end dsRNA, respectively (FIG. 7B). These rates are similar to the rate obtained in the cockroaches non-exposed to the sublethal dose of imidacloprid for 30 days (FIGS. 7A and 7B). Therefore, the dsRNAs, by modifying expression of the nAChRs, would appear to bypass the resistance mechanisms set up by the insect, by restoring their sensitivity to the insecticide.

These results also show the advantage of the technique for bypassing the resistance mechanisms set up by the insect against the insecticide, by targeting the nicotinic sub-unit(s) over-expressed by the resistant cockroaches, to restore the “sensitive” phenotype of the cockroaches.

### Example 3: Use of the RNA Interference Technique Targeting the Nicotinic β2 Sub-Unit of Acyrthosiphon pisum, by Ingestion in Larvae

### Materials and Methods

### Obtaining One-Day Old Larvae and Artificial Feeding System

All the experiments were conducted on aphid larvae aged one day. The adult aphids were collected after transfer and placed in an artificial feeding system comprising a PVC tube of 3 cm in diameter and of 6 cm in height, called a cell, which was closed at each end by a piece of Parafilm®. To nourish the aphids (10 adult aphids/cell), 200 μL of a nutrient solution were deposited on the Parafilm® at one of the two ends of the cell. The nutrient solution was then spread over the Parafilm® by overlaying a 2^{nd} Parafilm®. The aphids were therefore able to feed by piercing the Parafilm® with their rostrum. The cells were then placed in a box containing 6-well plates filled with moistened cotton wool to prevent breakage of the Parafilm® and to ensure optimal development of the aphids. The cells were finally placed in a temperature-controlled at 20° C. chamber for 24 h with a photoperiod of 16 h.

Preparation of the Lipoplexes Containing the dsRNAs and Administration Via Ingestion

Lipoplexes containing dsRNAs were prepared to limit degradation thereof in the aphid. For this purpose, 9 μL of dsRNA (dsRNA-LacZ or dsRNA-β2; 4.44 μg/μL) were incubated with 0.1 μL of Escort IV® transfection agent (Sigma-Aldrich) and 0.9 μL H₂O for 30 minutes at room temperature. So that the aphids ingest the dsRNAs via the artificial system, 10 μl of lipoplexes were mixed with 190 μL of nutrient solution.

Preparation of the dsRNAs and analysis were similar to those described in Example 1.

### Results

Evaluation of the Effect of Ingestion of dsRNA-β2 on the Expression of the Transcripts of the Nicotinic β2 Sub-Unit:

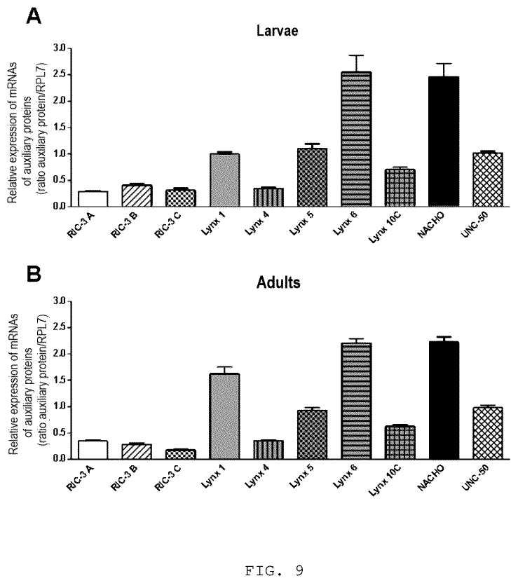

To evaluate the effects of the dsRNAs targeting the nicotinic β2 sub-unit (dsRNA-(2), quantification by qPCR of the mRNAs encoding this sub-unit was carried out from the cDNA of aphid larvae obtained at 2 h, 24 h, 48 h and 72 h after contacting them with a nutrient solution containing the dsRNAs of interest (200 ng/μL). At 2 h, the expression level of the transcripts of the β2 sub-unit of the larvae having ingested dsRNA-2 was reduced by 33% compared with larvae treated with the control dsRNAs targeting the bacterial gene LacZ (dsRNA-LacZ; *p<0.05, FIG. 8A). On the other hand, from 24 h to 72 h, no significant difference was observed in the expression of the mRNAs. These results therefore indicate that under these conditions ingestion of dsRNA-β2 efficiently decreases the expression level of the β2 sub-unit after 2 h.

Evaluation of the Use of dsRNA-β2 as Bioinsecticide

To validate the use of dsRNA-β2 as bioinsecticide, the mortality of A. pisum larvae was evaluated at 24 h, 48 h and 72 h after contacting the larvae with a nutrient solution containing the dsRNAs of interest (dsRNA-β2 or dsRNA-LacZ) at 200 ng/μL. The results are expressed in percent corrected mortality relative to the control condition (dsRNA-LacZ). The ingestion of dsRNA-β2 induces a significant increase in the mortality of the larvae of 19%, 32% and 33% respectively at 24 h (**p<0.01), 48 h (**p<0.01) and 72 h (*p<0.05) respectively, compared with the mortality in the larvae which had ingested dsRNA-LacZ (FIG. 8B). Therefore, dsRNA-β2 has a bio-insecticidal effect and their use can be envisaged for controlling the pea aphid.

This example particularly illustrate that the administration mode via ingestion in larvae is also functional.

### Example 4: Molecular Characterisation of the Auxiliary Proteins Regulating the Nicotinic Receptors in the Pea Aphid Acyrthosiphon pisum

### Materials and Methods

### Identification and Biocomputational Analysis of the Sequences of the Auxiliary Proteins

The protein sequences of i) Lynx, ii) UNC-50, iii) NACHO and RIC-3 of the aphid A. pisum were identified with BLASTp software (NCBI) from those of the migratory locust Locusta migratoria, of the fruit fly Drosophila mojavensis and the fruit fly Drosophila melanogaster, respectively. The sequence having the greatest identity was considered to be the reference sequence encoding the protein of interest and was therefore used to determine the corresponding nucleotide sequence in the Gene database (NCBI). The coding sequences were identified with Expasy software and alignments of the sequences were obtained with the Clustal Omega software to allow evidencing of mutations within the predicted sequences. Finally, the presence of the GPI anchor within the Lynx protein was predicted with the NetGPI software.

### Extraction of Total RNAs/Reverse Transcription: As Previously Described.

### PCR Amplification of the Coding Sequences of the Auxiliary Proteins

To identify the nucleotide sequences encoding the proteins of interest, namely NACHO, UNC-50, RIC-3 and Lynx, amplification by PCR was performed from cDNAs of adult aphids A. pisum, using a high fidelity DNA polymerase, KOD Hot Start™ (Novagen). Each reaction was conducted with 0.4 μL of KOD Hot Start™ DNA polymerase (1 U/μL), 2 μL of KOD reaction buffer (10×), 1.2 μL of MgSO₄ (10 mM), 2 μL of dNTP (2 mM), 2.4 μL of specific sense and antisense primers (5 μM), 2 μL of cDNA in 1:20 dilution and 15.6 μL of H₂O. The PCR experimental conditions were composed of initial denaturation for 2 minutes at 95° C., followed by 35 cycles each comprising a denaturation phase for 20 seconds at 95° C., a hybridization phase for 10 seconds at 45° C. (NACHO, UNC-50, Lynx 1, Lynx 4 and Lynx 10C) or 60° C. (RIC-3, Lynx 5 and Lynx 6) and an extension phase at 70° C. for 30 seconds (NACHO, UNC-50, Lynx 1, Lynx 4 and Lynx 10C) or 40 seconds (RIC-3, Lynx 5 and Lynx 6).

This step was followed by electrophoretic migration and purification of the PCR products, and thereafter verification of the nucleotide sequences by sequencing and analysis by quantitative PCR of the expression of the transcripts of the auxiliary proteins of interest.

### Results

### Identification of the Auxiliary Proteins RIC-3, Lynx, UNC-50 and NACHO in the Aphid A. Pisum

Since the genome of the aphid A. pisum is sequenced but scarcely annotated, a search for the sequences of the proteins of interest by screening databases is possible. Therefore, to determine the protein and nucleotide sequences of these proteins, biocomputational analysis using the BLASTp software (NCBI) was carried out on the database of non-redundant protein sequences of A. pisum (NCBI) using known sequences of the migratory locust Locusta migratoria for Lynx, of the fruit fly D. melanogaster for NACHO and RIC-3 and of the fruit fly D. mojavensis for UNC-50. Therefore, contrary to D. melanogaster which has 19 variants of RIC-3, A. pisum only appears to have three isoforms called RIC-3 A (398 amino acids), RIC-3 B (496 amino acids) and RIC-3 C (502 amino acids) the sequences of which respectively have 39%, 58% and 37% identity with that of the variants L, D and P of the fruit fly protein RIC-3. With respect to the Lynx protein, 5 isoforms called Lynx 1 to Lynx 10C have been determined whereas the locust has 11. These isoforms comprising 144, 162, 148, 156 and 143 amino acids, respectively have 40%, 45%, 65%, 45% and 25% identity with Lynx 1, Lynx 4, Lynx 5, Lynx 6 and Lynx 10 of L. migratoria. To confirm that the different isoforms of Lynx identified in A. pisum have a GPI anchor, the presence thereof was predicted (NetGPI software).

It was therefore evidenced that all the Lynx sequences in A. pisum have a GPI anchor. Screening of the NACHO protein allowed identification of 1 protein sequence in A. pisum composed of 152 amino acids having 57% identity with that of the fruit fly D. melanogaster. Finally, for UNC-50, only 1 protein sequence of 261 amino acids was evidenced of which 52% have identity with that of the fruit fly D. mojavensis.

Expression of NACHO, RIC-3, Lynx and UNC-50 in the Aphid A. pisum qPCR reactions were carried out to quantify the expression levels of the mRNAs encoding the auxiliary proteins NACHO and UNC-50, but also encoding the various isoforms of the proteins RIC-3 and Lynx in the aphid A. pisum (larvae and adults). Analysis of the expression of the various auxiliary proteins shows that the expression of Lynx 6 and NACHO is in majority in the pea aphid irrespective of its stage of development, contrary to that of Lynx 4 and the 3 isoforms of RIC-3 which remain in minority (FIG. 9). Among the 3 isoforms of RIC-3 identified in A. pisum, RIC-3 B would appear to be the majority isoform in the larvae, whereas in the adult aphids it appears to be RIC-3 A (FIG. 9). Regarding the isoforms of the Lynx protein, it is Lynx 6 which appears to be more expressed than the 4 other isoforms irrespective of the stage of development (FIG. 9).

The examples given above are only preferred embodiments of the invention and are not intended to limit the scope of the present invention. Modifications, replacements by equivalents and improvements made by persons skilled in the art without departing from the spirit of the present invention must come within the framework of the present invention defined by the appended claims.

### BIBLIOGRAPHIC REFERENCES

Gepts et al., 2005. Legumes as a Model Plant Family. Genomics for Food and Feed Report of the Cross-Legume Advances through Genomics Conference. Plant Physiol. 2005; 137 (4): 1228-1235
Whyard et al., 2009. Ingested double-stranded RNAs can act as species-specific insecticides. Insect Biochem Mol Biol. 2009 November; 39 (11): 824-32.
Dale et al., 2010. Identification of ion channel genes in the Acyrthosiphon pisum genome. Insect Mol Biol. 2010; 19 (s2): 141-153.
Niu et al (2019). Topical dsRNA delivery induces gene silencing and mortality in the pea aphid. Pest Manag Sci. 2019; 75 (11): 2873-2881.
Benzidane Y et al., 2017. Subchronic exposure to sublethal dose of imidacloprid changes electrophysiological properties and expression pattern of nicotinic acetylcholine receptor subtypes in insect neurosecretory cells. Neurotoxicology. 62:239-47.
Thany S H et al., 2007. Exploring the pharmacological properties of insect nicotinic acetylcholine receptors. Trends Pharmacol Sci. 28:14-22.
Sinakevitch I G et al., 1996. Anatomy and targets of Dorsal Unpaired Median neurones in the Terminal Abdominal Ganglion of the male cockroach Periplaneta americana L. J Comp Neurol. 367:147-63.
Grolleau F et al., 2000. Dorsal unpaired median neurones in the insect central nervous system: towards a better understanding of the ionic mechanisms underlying spontaneous electrical activity. J Exp Biol. 203:1633-48.
Jones et al., 2021. The cys-loop ligand-gated ion channel gene superfamilies of the cockroaches Blattella germanica and Periplaneta americana. Pest Manag Sci. 77 (8): 3787-3799

1. A method for controlling insect pests comprising inhibiting translation of mRNA of a target gene belonging to the cholinergic system of the insect pests, induced by RNA interference.

2. The method according to claim 1, comprising:
preparing a double-stranded RNA specific to the mRNA of a target gene; and
administering the double-stranded RNA to at least one insect pest in an effective amount to induce mortality, or sensitivity to an insecticide, of the targeted at least one insect pest.

3. The method according to claim 1, comprising:
preparing a single-stranded antisense oligonucleotide specific to the mRNA of a target gene; and
administering the single-stranded antisense oligonucleotide to at least one insect pest in an effective amount to induce mortality, or sensitivity to an insecticide, of the targeted insect at least one insect pest.

4. The method according to claim 1 wherein the mRNA of the target gene is chosen from the group consisting of mRNA of neuronal α sub-units of nicotinic receptor, mRNA of neuronal β sub-units of nicotinic receptor, mRNA encoding auxiliary proteins and molecules and isoforms thereof, and a protein of nicotinic receptor interactome.

5. The method according to claim 4, wherein the mRNA of the target gene is chosen from the group consisting of mRNA of neuronal α sub-units of nicotinic receptor of Acyrthosiphon pisum, mRNA encoding neuronal α2 sub-unit of nicotinic receptor, mRNA encoding neuronal α3 sub-unit of nicotinic receptor, mRNA encoding isoforms of neuronal α4 sub-unit of nicotinic receptor, mRNA encoding isoforms of neuronal α6 sub-unit of nicotinic receptor, mRNA encoding isoforms of neuronal α7 sub-unit of nicotinic receptor, mRNA encoding neuronal α8 sub-unit of nicotinic receptor, mRNA encoding neuronal α9 sub-unit of nicotinic receptor, mRNA encoding neuronal α10 sub-unit of nicotinic receptor and isoforms thereof, mRNA of the neuronal β sub-units of nicotinic receptor of Acyrthosiphon pisum, and mRNA encoding neuronal ββ2 sub-unit of nicotinic receptor and the isoforms thereof.

6. The method according to claim 4, wherein the mRNA of the target gene is chosen from the group consisting of mRNA of auxiliary proteins of nicotinic receptor of Acyrthosiphon pisum.

7. The method according to claim 4, wherein the mRNA of the target gene is chosen from the group consisting of mRNA of neuronal α sub-units of nicotinic receptor of Periplaneta americana, mRNA encoding neuronal α2 sub-unit of nicotinic receptor, mRNA encoding neuronal α3 sub-unit of nicotinic receptor, mRNA encoding isoforms of neuronal α4 sub-unit of nicotinic receptor, mRNA encoding neuronal α5 sub-unit of nicotinic receptor, mRNA encoding isoforms of neuronal α6 sub-unit of nicotinic receptor, mRNA encoding isoforms of neuronal α7 sub-unit of nicotinic receptor, mRNA encoding neuronal α8 sub-unit of nicotinic receptor, mRNA encoding neuronal α9 sub-unit of nicotinic receptor and isoforms thereof, mRNA neuronal β sub-units of nicotinic receptor of Periplaneta americana, mRNA encoding neuronal ββ2 sub-unit of nicotinic receptor, mRNA encoding neuronal β3 sub-unit of nicotinic receptor, mRNA encoding neuronal β4 sub-unit of nicotinic receptor, mRNA encoding neuronal β5 sub-unit of nicotinic receptor, mRNA encoding neuronal β6 sub-unit of nicotinic receptor, mRNA encoding neuronal β7 sub-unit of nicotinic receptor, mRNA encoding neuronal β8 sub-unit of nicotinic receptor, mRNA encoding neuronal β9 sub-unit of nicotinic receptor, and mRNA encoding neuronal β10 sub-unit of nicotinic receptor and isoforms thereof.

8. The method according to claim 1 comprising administering to the insect pests double-stranded RNA or single-stranded antisense oligonucleotide specific to the mRNA of a target gene via topical administration, by spraying, by vaporization, via nanoparticles, via feeding, via trapping in a bait box, or via crop irrigation.

9. The method according to claim 1, wherein the insect pest is at least one chosen from phytophagous insects, saprophagous insects, detritivorous insects, predator insects, parasitic insects, commensal insects, and hematophagous insects, and is from Helicoverpa armigera, Bemisia tabaci, Plutella xylostella, Tribolium castaneum, Myzus persicae, Spodoptera frugiperda, Aphis gossypii, Nilaparvata lugens, Spodoptera exigua, Ceratitis capitata, Cydia pomonella, Acyrthosiphon pisum, Diaphorina citri or Thrips tabaci.

10. The method according to claim 8, comprising administering by feeding the insect pests a transgenic organism expressing the double-stranded RNA or the antisense oligonucleotide.

11. The method according to claim 10, wherein the transgenic organism is a transgenic plant.

12. An insecticide composition for insect pests, the composition comprising a double-stranded RNA or antisense oligonucleotide and at least one from among a transfection agent and a solvent,
wherein said double-stranded RNA or antisense oligonucleotide comprises a nucleotide sequence having at least 90% identity with at least part of the sequence of a target mRNA, the target mRNA being chosen from the group consisting of coding sequences of genes of neuronal α sub-units of nicotinic receptor, genes of neuronal β sub-units of nicotinic receptor and isoforms thereof, genes encoding auxiliary proteins and molecules of nicotinic receptor and isoforms thereof, and genes encoding protein of nicotinic receptor interactome.

13. The insecticide composition according to claim 12 comprising the transfection agent wherein the transfection agent comprises a lipid compound, a liposome, a niosome, a lipid nanoparticle, a dendrimer, or an insect virus.

14. The insecticide composition according to claim 12, further comprising one or more agents chosen from a synergizing agent, a repellent agent, and an attractant agent.

15. The insecticide composition according to claim 12, further comprising a carrier acceptable from an agricultural, agri-food, health, and/or environmental perspective.

16. The insecticide composition according to claim 12 wherein the composition is formulated in the form of a bait for the insect pests.

17. A transgenic plant cell, plant tissue or plant comprising at least one nucleic acid transcribed to produce a double-stranded RNA, wherein the double-stranded RNA comprises a nucleotide sequence having at least 90% identity with at least part of the sequence of a target mRNA, the target mRNA being chosen from the group consisting of coding sequences of genes of neuronal α sub-units of nicotinic receptor and isoforms thereof, neuronal β sub-units of nicotinic receptor and isoforms thereof, auxiliary proteins and molecules of nicotinic receptor and isoforms thereof, and a protein of nicotinic receptor interactome.

18. The transgenic plant cell, plant tissue or plant according to claim 17, wherein the double-stranded RNA has a length of 20-2000 base pairs.

19. An interfering RNA comprising double-stranded RNA that inhibits translation of mRNAs corresponding to a coding sequence of any one of the genes of α sub-units of nicotinic receptor and isoforms thereof, of the genes of neuronal β sub-unit of nicotinic receptor and isoforms thereof, any one of the nucleotide sequences of genes encoding auxiliary proteins and molecules of nicotinic receptor, and isoforms thereof, or to a DNA sequence encoding a protein of nicotinic receptor interactome.

20. An antisense oligonucleotide, wherein the antisense oligonucleotide inhibits translation of mRNAs corresponding to the coding sequence of any one of the genes of neuronal α sub-units of nicotinic receptor, of the genes of neuronal β sub-units of nicotinic receptor, and the isoforms thereof, to any one of the nucleotide sequences of genes encoding auxiliary proteins and molecules of nicotinic receptor, and the isoforms thereof, or to a DNA sequence encoding a protein of nicotinic receptor interactome.

21. A method of using the interfering RNA according to claim 19 as bioinsecticide comprising administering the interfering RNA to at least one insect pest in an effective amount.

22. A method of using the interfering RNA according to claim 19 as agent for synergizing an insecticidal effect of an insecticide, or of a molecule with insecticidal effect, against an insect pest comprising administering the interfering RNA to at least one insect pest in an effective amount.

23. The method of claim 22 wherein the insecticide comprises imidacloprid, clothianidin, acetamiprid, dinotefuran, nitenpyram, thiacloprid, thiamethoxam, spinosyns, butenolides, mesoionics, sulfoximines, carbamates, pyrethroids, oxadiazines and organophosphates, and the molecule with insecticidal effect comprises a natural substance, an essential oil, or a pheromone.

24. A method of using the interfering RNA according to 19 as agent for restoring the sensitivity of an insect pest to an insecticide comprising administering the interfering RNA to at least one insect pest in an effective amount.


SEQUENCE LISTING (91 sequences)

SEQ ID NO: 1 - DNA, 2532 bp, Acyrthosiphon pisum
  misc_feature 1..2532: note = alpha1 sub-unit of the nicotinic acetylcholine receptor
  source 1..2532: mol_type = genomic DNA
cgcgaacacgtttcgacgaccgtccgttgtcgaccgttcgcgtcgggtgctataataggtacttatgtgcctatgcaatcatgtcgacggaggtaatgatgccattgaactccgagcacgttgtgtgtacaatcacttgatatcgtatcgtcgttctcgtccgcgatcaacaccaatcaactcattaacgttcgatttctcgcgcgatgaaaatcatttgtgcgatattcgcagcggtcattgtcggtcagttggccatgccgtacgcgtctgtgtatggaagtgccgatgccaaacgactttatgacgatctgctgagcaactacaacagactcatcagacccgtcggcaacaattctgatcggttgacggttaaaatgggtctgaaactttcgcaaatcatcgaagtgaatttaagaaatcaaattatgacgacaaacgtatgggtcgagcaagaatggaatgactacaaactgaaatggaatcccgaagattacggtggggtagatactctacacgtaccatccgagcacatttggttgccggacatagtcttgtataataacgccgatggcaattacgaggtgacgataatgacgaaagcgatactgcattacacggggaaagtcgtgtggaaaccgccggccatttacaaatccttctgcgagataaacgtagagtacttcccgttcgacgagcagacgtgttccatgaagtttggatcttggacttacgacggatacatgatggacttgcggcacatctcccaggcgcccgactcagacgtgatcgaagtgggcatcgacctgcaggactactacctgtcggtggagtgggacatcatgggcgtgccggccgtgtggcacgaaaagttttacgtgtgctgcgaggaaccgtacctggacatattcttcaacatcaccctccgtaggaagacgctgttctacacggtgaacctgatcataccgtgcgtgggcatatcgttcctgtccgtgctggtgttctacctgccgtccgagtccggcgagaaggtgtcgctgtgcatatcgatactgctctcgctcaccgtgttcttcctgctgctggtcgagatcataccgcccacgtcgctcaccgtcccgctgctaggcaaatacctgctgtttaccatggtcctggtcacgttgtccgtgttcgtcaccgtggccgtcctcaacgtcaacttcaggtcaccggtaacgcacaagatgagaccgtgggtggtgaagctgttcatccaaatcctgcccaaggtgctgttcatcgagcggcccaagaagggagactcgatcgacgaggacgacgacgacgatgacgaaaagaatggtgaaatcctgtcgggcgtgttcgacgttccgtctgagatcgacaagtacttgggctacaaccgtggttacagtttcgattacgatgtaccaccgccgctgccgtcgtccaggtacgcatgcgccagggccacgtgccccgggggggtgagcggagtcgcgggcgtgagcgccggtgcgggagccgtcaccgggtccaacgacacggtggtgaacatggcgtcggacgaggacgaggacgcgatcgagctcgacgccgaggacgagtacgatgacatgttcagcccgacgaccacgacggacgacggtctggtcagcccgacgttcgagagccatcaccaccatcaccagcaccagcacgggtgtccggtggaccaacagcccaggcacgatccggccatgcagactctccaggacgcaaaatttattgctcaacacgtcaagaaccaagacaagtttgacgagatcatcgaagactggcaatacgtggccatggtcctggacaggctgttcctatggatattcacgtgcgcctgtctcatcggcacagccctgatcattttccaagcgccggccctgtacgacaagacaaagcccatcgacgttgtttactcgaaaatcgcgaaaaagaaactgcaagccattttatgagacactacaacaatacaaagctcaatgacgttttttagtaaacaacttagtgaatacaaacagttaatcgataacaatcctataataatgtataaattatacttcgttttactaatcatcaagtttttattatacattatatttattgtttttttataattgatttttcatgaatggcgtgaaacgttgtcgccaataaaataatattataatatcaataataataattagcgttttacagttcatattttatttgttataaataaattattgaaaatcattagattcaatatagccattgaacttggtgccatcagttatcatattaatattataaaattgtataacttatattatattgttattatttgatatttcgtttaatacagagactaagtataataatatatatgacccgcatttgtttaaaatattcaactatctagttgaactatttcactaagttattcagataaatgctttgtcattgcaactcatgtcatttggtttcttgaaaataaacgaatgatataataaa

SEQ ID NO: 2 - DNA, 4357 bp, Acyrthosiphon pisum
  misc_feature 1..4357: note = alpha2 sub-unit of the nicotinic acetylcholine receptor
  source 1..4357: mol_type = genomic DNA
atgccgtccgtcgcccccgccgaggctcacgacggcgacatcagccacaacaacaacaacaacagcagcagccgcaacaacaaaaagttccggggacgacgccaccaccggggctgctcgcagctgacgtcgtccctgccgctgctgctcctgttcgccgtcgggtgcctgtgcaacccggacgccaagcgcctatacgacgatctgctcagcaactacaaccggctcatccggccggtcagcaacaacaccgacaccgttctggtcaagctgggcctcaggctgtcgcagctcatcgagttgaatctcaaggatcagattttaacgacgaacgtatggctggagcacgaatgggcagaccacaaattcatttgggaaccactggagtacggaggcgttaaagagctgtatgtgccgtccgagcatatctggctgccggatatcgtcctttacaacaacgccgacggagagtacgtgatcaccaccatgacaaaggctgtgcttcatcactccggcaaagtgatgtggacaccaccagccatattcaaatcgtcgtgcgaaatcgacgttcgctatttcccgttcgatcagcaaacatgttttatgaaattcggctcgtggacgtacgacggaaaccagataaacctcaagcatatcggccaattggtgggatccaacaaagtggacgtgggcatcgacctgtcagcgtactacccgagcgtggaatgggacattcttggcgtgccggcggagagacacgagaagtactactcgtgttgcgttgaaccttacttcgacatattctttaacataaccctgcgacggaggacgctgttttacaccgttaacctaatcgtgccctgcgtgggcatatcttacctgtcggtgttggtgttctacctgcccgccgactccaaggagaaaatttcactgtgcatcacgatcctcctgtcccagaccatgttcttcttgctcatctccgaaatcataccgtcgacgtcactgtcgttgccgctgctcggcaagtacctgttgttcaccatgttgctggtagccctgtgcgtggtcgtcaccatcatcatcataaacatacattacaggcaaccgagcacgcacaagataccgtcgtggatgcggacggtgttcatcagagcactgccaaaattgttgctcatgagggtgcccgaacagctgttggccgattcagcgatgaaacagaagcagatgaagacgtgcaaaaaattaaaccttaacttggccaggttgaacgcggccagtgtgggtgccacgctgaacgttcctccggcgccggtcgcttcgccgcccgccggccacggatcacccaccgactcgctgcgccgttacgtggcggccgccggccggaccggttgcaacggaacggcggccgcggccggagcggccagccggctggtgaacgcggccgtggcgtccatcgacgacacgctcaacgaagtgccggccgccatcaggaagaagtatccgttcgagctggagaaggccatacacaacgttaagttcattcagcaccacttgcagcgccaggacgaatacaacacggaagatcaagactggggcttcgtggctatggtgctcgacagactattcttgtggatattcacggtcgcgtccattatgggcacaattctcatcctgtgcgaagcgccagcgctctacgatgacacgaagccaatcgaccgggacctgtcgttcatcgctagaaagcaattttccccgacgtccgatttggaatagtccgtcgtatgacattaggttataattttcacgtacaagcgaaaaaatgatcaaagcgtgaaatacagttgcagtgatggacctcgacgttaaacggttgatagtaatattattttaacgagtcgtcaatatcgttacaacattattgctaaaatgtctcagaagttttaggttatggattttttaccgttaagtattattatattttgcaccaatttaagttgtcggaatattatgttataatgaatggtgttttaaaataaataaatcattaaacgctttttaatagcatagtacttataggtactaaaaattcacaacattacatttagattaaaaaaaaaaaacaattcgttcatttttttttttaaattcaatctcacattttcgtattggttttgttatctattttttaaaacgtttgtagtaatattattatatagtagacaataacttgtttgtttaagctaataatataatattataataatatactgacaggtttttaaaaatatttccatgtaattgttgtacataaaataaaatacaatcgtcgtaagtattaataataatgtacacgttgtacgcaacaagaaattgttatttccaggtacctaaccatattatgcaaaacacaaagaaatactttttatgaactatatgatataggtaggtaggtattatagttgtaatatttatgtgaactattcgcaaacaattatttatgatttcaacaggttgtataagtacgtatattaacgtttcatttttttataataacaaagcttaaacgatgcgcttgaaattaaaaatgaataacacttgcaaaaaaaaatgatattataataatattatcactacgtatacgtttcatggtgtgaagtaatatttataaattgcgcgcaaaacgtaggtataacaataaaattcatcgatttaactataaccgcttcaatatttgctgaaatttggcactcgtgccgaataaatatttagtacaaaattaaattttgatatatgtattaacacgactttctttgtccatttaattttttattctattaagtccaaaatattacgtcttatattatctgtcttaattttttttaaaattattatcagaatcacagacatctcgtaataattataatatgtaagtaatatttatgaaccagaatacccgatcatgtcgtattaacgtcacaaacatgcgaaactcgaatgatttgtattttaatttatcttttttaattaaagatattattattattatactaatttttataattaaatttttttttcccacacacactcgcgtttaaagatgagtccaagtggataagctctcttggagacatcaataaaaaatatctatttaaaataaaacatagtttacgtaattaccagctagcttattattactagctataagtttaacgcgaaatcgttttctcgggatgtttaaaaaatgtagacaccaccgttatagatagtggctcctttcttgccgccatctttccctatcctttgcaacttctcaccatcgtatattattttggttccacggctttaattttttttttcgatgcgatcctccgatcgtaaacgcgatatgcctataacagtgttttcccaatcggatcatcttttaaaccaccttactaaatgagctccgctccttcctccagcccacataaaactttttctcataatttctgcaatgataaggtacctagtggtcatatataaacgtttatttaaaatttttcaacaataaggtagcagcgatttttttttggagattaaaaccaaagtctaatatgccaaatgtaagttttaatttcgacacaatttgtttgtaataaaacaaaggataacaacgttgcacgctactctgtgtttttctcatgcaaggaaaatctatgaatcataagtatgcttttgagggggagggaacaataataaacgttatacgtgttacgaaaatcggccccgtgaaaatctgtcagaaaaaaagtcgtagccagcccaaaaacatatttatgttaaaatagctaaaatgcgtattttatatgctaattttgtcacaattatctacttaaaaataaaaacgttataatcgtggtaaggcgaaaggtcatgatgaataatttgcattatcaataaattatcgttaataattaaaccgtatacgataatattataatactgacttatcctaccaggataaatagacaaaaacaatatacctaatatctacatgttaaccgtgtgcgctcattcgatcgatttgcatagtattattattgcgaataagcttcgggccgtccgttatcaatttaaatctgttttaagaaaataacttcgtcgtcgccgttttacgattttttaacgagtgcccgctattatacgcgattattctatgctgaccttctaactagtttcttcccacgaagcagtaccgattttttcagacacaatattattatataataacgacgaccacgatcgctagatatacagttatgttatgtcttaacgggtttttctttttttttttacaacagaactcgtgtaagtagtg

SEQ ID NO: 3 - DNA, 2649 bp, Acyrthosiphon pisum
  misc_feature 1..2649: note = alpha3 sub-unit of the nicotinic acetylcholine receptor
  source 1..2649: mol_type = genomic DNA
gtacgccgtggcacacgacgcgagtccgacgcccgaacgccatgcccgacgccgccgccgctgtcgccgccgtcgtgtcagcgccgccaccgccgtcgtggtcgccgcgtaacgccgccgtggtcgccgcaaacgaatgagaaaacgcacgaaaatcgcgtggtgcgtaaaataataataataataattatactgccgcagtcgctggtgaatctttccggagattctgcagctcctcgaccccccgacgttttgctaatgacatcggttaccgtcgtatacctattatagtgacactgtgtgtaatgataatatattataataataatacctatatacttttgtacgttaaatgcgacgataatattattcgataaataggaatcacaaataatcattcgttaatgacaaaaataatataacacacttctggtcccacgtgcacagtttcaggaaaaaatgcgattgtgcgacctcatgctgtgacctttgatatggtcaccgaaaaatgaagacgttggccggcgccataatactatgcatcatgctcgtcataatttccggatgttcaatgaatccggacgcgaaaaggctttacgacgaccttttatcaaattacaacaaactcgtaaggcctgtgctcaacaacactgatccgctaccagtacggattaagctcaaactatcacagctcatcgacattaatctaaaaaaccaaattatgacgacgaacttatgggtggaacagtattggtatgactataaattgacgtggaatcctgaggaatacggtggtgtcgaagggttacacgtaccatctgaacacgtatggaggccagatattgttctttataacaatgctgacggcaatttcgaagtaactttggccactaaggctatgcttcactatagtggtcgggttgagtggaaaccaccagctatttacaagtcttcctgtgaaatagatgtagaatttttcccgtttgacgaacagacgtgtgtgatgaaatttggctcgtggacctacgatggtttccaggtggatttgagacatgcaaatgaagtcagtggttcacgagttgtggacgtcggtgttgatttatccgaattttacgcgtccgtcgaatgggatattttagaggttccagcaataagaaatgagaaatattatacttgttgcgaagaaccatacttagacattacattcaatatcacgatgcggaggaaaacgctattttacacggtgaatttgatcataccttgtatggggatatcgtttcttacgatacttgtgttttaccttccatcggatagtaacgaaaaagttagtttaagtatttcgattcttctttcgctgaccgtgttctttctgctgctagccgaaatcattccgcccacatcactagtcgtgccacttttgggaaaatttgtgctatttacaatgatacttgacacgctcagtatatgtgtgacagtcgttgtgttaaatatacatttccgatctcccactacacacgtaatgtcaccatgggtccgccgagtattcattcacattctaccgagaatgttaatcatgagaagacctcactaccaaatggaaaggaagagtctgatgggaacatgtcatcgtattatggtgaggacgtgcaacgggcttgagcttagagatcaaaattcagccgatcagttgttgcacagcaacgatgtatttcctgccagggatttggaaagcggtgtaattggctgttgcgagattcacggtccaataatagcattacccagcccatccgaaacaaaccaaaatggcccagttcctaagggtagaaaacattggcacgattgtccagaactccataaggccgtcgagggcgctgcgtacatagctgattacatcaaaaaagaagaagaagagaaaaagattaaagaagattggaaatatgtggcgatggtgctggataggttgtttttgtggatatttacattggctgtgaccatgggatctgcagggataatcctacaggcacccactctgtatgacaatcggttgccaatcaatgtccggttgtcggaaatacttgcccatacaccaacagtgaaacagaattataataacattatgtaaagacgataattattgctcaatatggtcaacattggacaagccagtgacatccaacactggtctggaagagcgaacggtagagtcctacaaccaccactgcaactgggatataacacatatatataagcattatagttaataaaatgcaggatgagggaccaacaaatcgaggactgctggtcgtaatatgtacaatagtgtcaattctgatattagatactattttcatgttctcaaaattagtgaatcaaatagatagatatttttagataatttgtttataaatcaaatatttataagcagtataatatatatttatttaaaaaaataaaaccataaaataggacctgaattggttgtaagagaaatattaattttctcaactattatttgtcttgtaaaaagaaacatataattcatgttattatttattataataaaagaaagtaataacacttcatagttatatgtacatagaattatgttaacttgtgaaaaataaatgaatatatttgatgacgtttaaa

SEQ ID NO: 4 - DNA, 2483 bp, Acyrthosiphon pisum
  misc_feature 1..2483: note = alpha4 sub-unit of the nicotinic acetylcholine receptor
  source 1..2483: mol_type = genomic DNA
gtggcgcatttcagtagggccccacactcggcgtactctgtcgcgcacacccgtccgccgcgtacctacactgtcactgtacgctgctcccgcgaactcgtgtttatcgtcgcactgaccaccctcaacgaccgccgccacacatctcgctgaaaataatttcgtcgttttttatttaattttccgtcgtcgcagttattgccttcccaccaccattgttgtcgtttgtttcaactcgttcactgcacttgtgtcgtattatattgtaatgatatcataacaataaaaaaaatatcacgtacgaatataacaatataataatattatataatagtgtctcgcccgaaaccgtacggctacgccgccgtcaacagccccggatcggtcctcagatatgtggccgaaatttgcatttctcggactcatgattatccggttaatggtatctgcgaatccggatgcaaagagactgtacgacgatctcttgtccagttacaacaagctcgtgagaccagtggtgaacaccagtgatgttctacgtgtgtctataaaactgaaactgtcgcagctcatcgatgtgaacttgaaaaatcagattatgaccaccaatttgtgggtagagcaggcgtggaatgattacaaattgcaatgggacccgaaggagtatggtggcgtgcaaatgctccacgtgccttccgatcacatttggcggccggatatcgtgctctacaacaacgccgacggcaatttcgacgtgacgctggccaccaaggcgaccatataccacgtagggctggtagaatggaaaccgccggccatattcaaatcgtcgtgcgagatcgacgtggaatttttcccgttcgacgagcagacgtgcgtgttgaaattcggtagctggacttacgacggattcaaggtagacttgagacacatggacgaaaaggtcggttcgaacatcgtcgacgtcggagtggatctttcagaattttacatgtcggtcgaatgggatatactggaagtaccggcggtcagaaacgaaaagatctacccatgttgtgacgaaccgtatttggacataacgttcaacatcaccatgagaaggaaaacgcttttctacacggtaaacatcatcattccgtgcatgggcatctcattcctcacagttttgacgttctacttgccatcggacagcggtgaaaaggtgacgttgtctatatcaattttgattagtctccacgtttttttcttgctggttgtcgaaattatacctccgacgtcattagtagtcccattactgggaaaatatttgatatttgctatgatactggtttcaataagcatatgtgttacagtagtcgtattgaacgttcatttccgatcacctcagacgcacaaaatggcaccttgggtaaaaagagtgttcatccatatactgccaagacttttagtcatgcgaagacctcagtaccagtttgaaccaaacaaattcaacgccggttcgataatgctgaagggcagagacatacatccgtgtttcttccaggctggctgcggctacgacagcacggaacccggaaaagataaaagtcgtcgacaaacagacgacagcagcgtgtcgaaaggcaccagagaaacgtctaaagaagacttatctagtgacggcgaagggggaagctgtcagatacacggagtggcgcagagtctgatttccgacgagatatcagtcaccgaaatgactactgactgcagcaagagctcgatcctgctcaggagccccggatttctgcattcgtcctgtcctccggccatgcacaagtcgtgtttctgcatccgattcatagcggaacacacgaaaataacggacgatgcgaccaaagtaaaagaagattggaagtacgtcgcaatggtcttggacagactatttttgtggatattcacactggccgtgttcgcgggcacggcgggtattatcctacaagcacccacgctgtacgacgaccggataccgatcgataaaaaattcagcgaataccaaacatatacatcgtacagtctgacgcaacgcgagggcacctaaaaacaacgaaaaaattcaaattccaacgccgatgacgtcatttttgtagttgcagtttcagttgcagcggttgtatatgaataaaatattatgatgataataataataataataataataataataataagttagcaaaaaaaaaaaaatgtaaaaaaaaaattataattatgttacgttttgtatacgtttaaattttgttgtatattaagttttgttataatactgataacgatgatttcaatgatgcccagtaacagagtaatttattacgcgataataataatgataattcacagacactgcagtacgcgatgtgcccaatataaaaaatattatattattaattattattataatgataaaatgtatgattattatttattattattaatttattgttttg

SEQ ID NO: 5 - DNA, 2532 bp, Acyrthosiphon pisum
  misc_feature 1..2532: note = alpha4 sub-unit of the nicotinic acetylcholine receptor
  source 1..2532: mol_type = genomic DNA
gtggcgcatttcagtagggccccacactcggcgtactctgtcgcgcacacccgtccgccgcgtacctacactgtcactgtacgctgctcccgcgaactcgtgtttatcgtcgcactgaccaccctcaacgaccgccgccacacatctcgctgaaaataatttcgtcgttttttatttaattttccgtcgtcgcagttattgccttcccaccaccattgttgtcgtttgtttcaactcgttcactgcacttgtgtcgtattatattgtaatgatatcataacaataaaaaaaatatcacgtacgaatataacaatataataatattatataatagtgtctcgcccgaaaccgtacggctacgccgccgtcaacagccccggatcggtcctcagatatgtggccgaaatttgcatttctcggactcatgattatccggttaatggtatctgcgaatccggatgcaaagagactgtacgacgatctcttgtccagttacaacaagctcgtgagaccagtggtgaacaccagtgatgttctacgtgtgtctataaaactgaaactgtcgcagctcatcgatgtgaacttgaaaaatcagattatgaccaccaatttgtgggtagagcaggcgtggaatgattacaaattgcaatgggacccgaaggagtatggtggcgtgcaaatgctccacgtgccttccgatcacatttggcggccggatatcgtgctctacaacaacgccgacggcaatttcgacgtgacgctggccaccaaggcgaccatataccacgtagggctggtagaatggaaaccgccggccatattcaaatcgtcgtgcgagatcgacgtggaatttttcccgttcgacgagcagacgtgcgtgttgaaattcggtagctggacttacgacggattcaaggtagacttgagacacatggacgaaaaggtcggttcgaacatcgtcgacgtcggagtggatctttcagaattttacatgtcggtcgaatgggatatactggaagtaccggcggtcagaaacgaaaagatctacccatgttgtgacgaaccgtatttggacataacgttcaacatcaccatgagaaggaaaacgcttttctacacggtaaacatcatcattccgtgcatgggcatctcattcctcacagttttgacgttctacttgccatcggacagcggtgaaaaggtgacgttgtctatatcaattttgattagtctccacgtttttttcttgctggttgtcgaaattatacctccgacgtcattagtagtcccattactgggaaaatatttgatatttgctatgatactggtttcaataagcatatgtgttacagtagtcgtattgaacgttcatttccgatcacctcagacgcacaaaatggcaccttgggtaaaaagagtgttcatccatatactgccaagacttttagtcatgcgaagacctcagtaccagtttgaaccaaacaaattcaacgccggttcgataatgctgaagggcagagacatacatccgtgtttcttccaggctggctgcggctacgacagcacggaacccggaaaagataaaagtcgtcgacaaacagacgacagcagcgtgtcgaaaggcaccagagaaacgtctaaagaagacttatctagtgacggcgaagggggaagctgtcagatacacggagtggcgcagagtctgatttccgacgagatatcagtcaccgaaatgactactgactgcagcaagagctcgatcctgctcaggagccccggatttctgcattcgtcctgtcctccggccatgcacaagtcgtgtttctgcatccgattcatagcggaacacacgaaaataacggacgatgcgaccaaagtaaaagaagattggaagtacgtcgcaatggtcttggacagactatttttgtggatattcacactggccgtgttcgcgggcacggcgggtattatcctacaagcacccacgctgtacgacgaccggataccgatcgataaaaaattcagcgaataccaaacgtcacctagcataaggaagaaactgaagataggccgttattgaaataaaatatacatcgtacagtctgacgcaacgcgagggcacctaaaaacaacgaaaaaattcaaattccaacgccgatgacgtcatttttgtagttgcagtttcagttgcagcggttgtatatgaataaaatattatgatgataataataataataataataataataataataagttagcaaaaaaaaaaaaatgtaaaaaaaaaattataattatgttacgttttgtatacgtttaaattttgttgtatattaagttttgttataatactgataacgatgatttcaatgatgcccagtaacagagtaatttattacgcgataataataatgataattcacagacactgcagtacgcgatgtgcccaatataaaaaatattatattattaattattattataatgataaaatgtatgattattatttattattattaatttattgttttg

SEQ ID NO: 6 - DNA, 2381 bp, Acyrthosiphon pisum
  misc_feature 1..2381: note = alpha6 sub-unit of the nicotinic acetylcholine receptor
  source 1..2381: mol_type = genomic DNA
gtcccggcggctagtcgtgtatgagccgccgccactgacaccgctgttcgtcgtagtcgtcgtcgtccgccgcggccgacgtgtccgcagctcgtccactgaagacgccgccgccgtcgtagtcgtcttaccagtcggtcccatcgtaatcttcgactacaggtttttcgggttcttttgaattttttttcaccgtcttttctccctccaccctcttcctccttcccctttttacctcgtcgttccatttctcgcccgcgtgtaaatgatataatacacacacgtcgcgtatcacgcgttccgtgtaataataaccgcgtgtggaacccacctacataccgtcgccgtcgtttatcttggtttctctcgcgttaccaccgccgtgtcgccgcggtacaccgcgcaacaaatacgccggtgccggtaatgggtggccggcgcgccggtaacgccaccgcgagccgtccgcgtcgtcgccgccgtcgaccgcacgacggacagcgttaacgaccatcgctatcacgaccactgtcgtcgccgtcgccgccgtacgctgagacacgccacaagagccacaggccgcggtagcgatgcccgcgatgattcggtgcaactggtggccgtgtcgccggcggtccgtccgacggtccgttcgccccgagccagtcgtcccaccgcgaagttgttgtcgctgcgtaccagcgtgattgtcgtcgtcgtcgtcggttcttgccacgcgcgcaccgtccacacgagcgatgacgtcgtccttggcaaagtttgtcgccgtgctgttcctgttggcggccgtgaagataaaagaaagcgttcaaggtaaattcgagaggcaactcttggatgacctcctaatagattacaaccctctggagagacctgtatccaacgaatccgatccactcgaagttacatttggcatcacgctacagcagataatagacgtggatgaaaaaaatcaaatcgtcataacaaatgcgtggttaaacatggaatgggtggattacaatatgaggtggaacatttccgaatatggcggtgtcaaggatctcaggctcaatccaaaccgactgtggaaaccagacatactcatgtataacagtgccgacgaaaaatttgatggtacttatcaaaccaatgtggtagtgaggcacaatggtacatgcctgtacgtgccaccgggtattttcaaaagtacgtgtaagatcgacatcacatggttcccatttgatgaccaaaattgccttatgaagtttggtagttggacatacaacggttttcagattgatttagtgctcaaatcagatggcggcgatctatcggactttacaacgaacggtgaatggtatttgctaggtatgcctggaaaaaagaacgtgataaactacgcctgttgcccagaaccgtacgtggacatcacgtttaccataaagattagacgaaggacgttatattatttcttcaacctgatcgtgccgtgcgtgctaatctcgtcgatggctctcctgggctttacgctgcctccggactgcggcgaaaagctcacactagaaataactatactactgtcactaacagtatttctgaatctggtcgccgagtcaatgccaaccacgtccgaagcggttccccttataggtacatacttcaattgcatcatgtttatggtggcttcgtcggtagttctcacattggtggtgctcaactatcatcatcgaacagctgacatacatacaatgggaccatgggttaagtgctttttcttgctatggttgccgtggatcttgtgtatgcaaaggccagggaaagacatatcgttcaaagccatcagaagtagatatacagatcggagaggcatcgaactccgggagcgatcgtcgagaagtttattagccaacgttcttgatatcgatgatgattttcgagcgagcgttctatcacaccgcgtgtacaacaacggggctgccagttcgtcgtcaccggcgtccgagtcgagaagcgtctgcaactactcggccagagaattgcaactcatattgcaagaggtgcggtacatcactgaccacatgcgaaaacaacaggacgaccaagaagtgatcaacgactggaaatacgcagcgatggtagtggacaggttctgtttgatagtgttcacttttttcacattagtcgccactgttgccgtaatgtattcagcgcctcacataattgttgagtgactcagacattgtgttcacttcttggatgtcgatatggcagtcatgattttgttaaatatttatttcagagagtatgagttgttgttttgaaatataatattaaaagtttttttttgtaataccataaaaaaatcgacgaagagctta

SEQ ID NO: 7 - DNA, 2381 bp, Acyrthosiphon pisum
  misc_feature 1..2381: note = alpha6 sub-unit of the nicotinic acetylcholine receptor
  source 1..2381: mol_type = genomic DNA
gtcccggcggctagtcgtgtatgagccgccgccactgacaccgctgttcgtcgtagtcgtcgtcgtccgccgcggccgacgtgtccgcagctcgtccactgaagacgccgccgccgtcgtagtcgtcttaccagtcggtcccatcgtaatcttcgactacaggtttttcgggttcttttgaattttttttcaccgtcttttctccctccaccctcttcctccttcccctttttacctcgtcgttccatttctcgcccgcgtgtaaatgatataatacacacacgtcgcgtatcacgcgttccgtgtaataataaccgcgtgtggaacccacctacataccgtcgccgtcgtttatcttggtttctctcgcgttaccaccgccgtgtcgccgcggtacaccgcgcaacaaatacgccggtgccggtaatgggtggccggcgcgccggtaacgccaccgcgagccgtccgcgtcgtcgccgccgtcgaccgcacgacggacagcgttaacgaccatcgctatcacgaccactgtcgtcgccgtcgccgccgtacgctgagacacgccacaagagccacaggccgcggtagcgatgcccgcgatgattcggtgcaactggtggccgtgtcgccggcggtccgtccgacggtccgttcgccccgagccagtcgtcccaccgcgaagttgttgtcgctgcgtaccagcgtgattgtcgtcgtcgtcgtcggttcttgccacgcgcgcaccgtccacacgagcgatgacgtcgtccttggcaaagtttgtcgccgtgctgttcctgttggcggccgtgaagataaaagaaagcgttcaaggtaaattcgagaggcaactcttggatgacctcctaatagattacaaccctctggagagacctgtatccaacgaatccgatccactcgaagttacatttggcatcacgctacagcagataatagacgtggatgaaaaaaatcaaatcgtcataacaaatgcgtggttaaacatggaatgggtggattacaatatgaggtggaacatttccgaatatggcggtgtcaaggatctcaggctcaatccaaaccgactgtggaaaccagacatactcatgtataacagtgccgacgaaaaatttgatggtacttatcaaaccaatgtggtagtgaggcacaatggtacatgcctgtacgtgccaccgggtattttcaaaagtacgtgtaagatcgacatcacatggttcccatttgatgaccaaaattgccttatgaagtttggtagttggacatacaacggttttcagattgatttagtgctcaaatcagatggcggcgatctatcggactttacaacgaacggtgaatggtatttgctaggtatgcctggaaaaaagaacgtgataaactacgcctgttgcccagaaccgtacgtggacatcacgtttaccataaagattagacgaaggacgttatattatttcttcaacctgatcgtgccgtgcgtgctaatctcgtcgatggctctcctgggctttacgctgcctccggactgcggcgaaaagctcacactaggagtgaccatcttgctatcactgacagtgttcctgaatttggtggcggaaacgcttccacaagtctccgacgcgatacccttgctaggtacatacttcaattgcatcatgtttatggtggcttcgtcggtagttctcacattggtggtgctcaactatcatcatcgaacagctgacatacatacaatgggaccatgggttaagtgctttttcttgctatggttgccgtggatcttgtgtatgcaaaggccagggaaagacatatcgttcaaagccatcagaagtagatatacagatcggagaggcatcgaactccgggagcgatcgtcgagaagtttattagccaacgttcttgatatcgatgatgattttcgagcgagcgttctatcacaccgcgtgtacaacaacggggctgccagttcgtcgtcaccggcgtccgagtcgagaagcgtctgcaactactcggccagagaattgcaactcatattgcaagaggtgcggtacatcactgaccacatgcgaaaacaacaggacgaccaagaagtgatcaacgactggaaatacgcagcgatggtagtggacaggttctgtttgatagtgttcacttttttcacattagtcgccactgttgccgtaatgtattcagcgcctcacataattgttgagtgactcagacattgtgttcacttcttggatgtcgatatggcagtcatgattttgttaaatatttatttcagagagtatgagttgttgttttgaaatataatattaaaagtttttttttgtaataccataaaaaaatcgacgaagagctta

SEQ ID NO: 8 - DNA, 5435 bp, Acyrthosiphon pisum
  misc_feature 1..5435: note = alpha7 sub-unit of the nicotinic acetylcholine receptor
  source 1..5435: mol_type = genomic DNA
ccgccaccaccgccgtagctgtagccgtagccttcgtcgtcccgccacccgacgcaatacgaaacgcggtggtgcacgcgccgcccgggcagaaacacgggcgcgcgctacggcgagcaaatatagaggtagtatacgcgggcggtacggggcggggtctggaccgccgcggagaattgtgcgcgcgcgcgcgcgctcgtacggtcgcttgacggcagggcgaaagctagcggcacgcaccatcagcaccaccgacagcgacggcagcagcggcaggaccagcatttggagcagccagccggcggttggaacggcagtattgtggtagtggcgcatttggacgcacgtctgtgtgtgtgtgtgtgtgtgtgtgtgtttgtgtgtgtgtgtgtgtgtgcgtgtgtgtgtgtgagcgtgtgcgtgtggaagagagagagagtgagagagagagagagagataaagagagaaaaagctgcgcgtgcgtgcgtgggtgggtgtgtgtgtgacggcgcgcgccaaagacgttccgccgagtacatagggacaggttttcgtacacagtcgtaccgcggttaccgctgtcgcaataccgcaacgaaaacagctcatcaatataatattattattattatacgggcatcccataaaaatatattacatagtttccgtggacgagacgtcgtcgtcatcgcctgccgtcgtatcacactagctgccttcaccttaggaaccggctcccatccgttgaacagtcgcggacgctcgctgccgccgctctctgcagagccgctgcgaggtttttccgtctctatttttttccggcacgtgccacaccgaccacggacggcactgttccggtccaccactaccaccgttgaaatggacgcgacggtcggccaccgtctcgcggatacccatcggcgccacaacaccggcgacagacccggcgaccagcagctggtatacggccgtcagcgacgacaggatgacggacgtcagaaggagccactgatcgttgttggccacaacgcgtcctgttgttgataggagaccggacgtcgacgacgaggacgggtcgcgcgcgccaccgcaaccgccgtcgccgccgctgaatgggacatgggaatactcccgtatctgctcatcgtctacctgatcattgcagaaacgctatgcggacctcacgagaagcgattgctcaacgatttattagacccttacaatacactagagaggccagtagctaatgaatcggagccacttcaacttagtttcggccttatgctcatgcaaatcatagatgtggatgaaaaaaatcagctgttgataaccaacatgtggttaaaattggaatggaacgatgtcaacctcaggtggaaccatagtgactatggtggtgtaaaggatttgagaataccgccgcataagatatggaaaccagatatgctcatgtataatagcgctgacgaaggtttcgacggcacgtacgcaaccaacgtggtggtcaacagcaatggtagctgcgtattcataccaccgggtattttcaagagcacgtgcaagatcgatatcacgtggttccccttcgacgatcaaaaatgtgatatgaaatttggcagctggacatacgacggatttcagttggatcttcaactccaagatgatcatggaggagatatcagtggttttattactaacggtgaatgggacctgcttggcgttcccggaaagcgcaatgtgatctactacagttgctgccccgaaccgtacatagacataacgttttccatcctaattagaaggcgcaccttgtattactttttcaacctgatagtcccgtgcgtgttgattgcgtctatggccgtactaggatttactctaccgccggactgtggcgaaaagctatctctaggtgtaacgattctcctgtcgttgaccgtttttctaaacatggtggccgagacaatgccggctacttcggacgcagttcctcttttaggtacttatttcaactgtatcatgttcatggttgcatcgtcagtcgtgtccacaatcatgattctgaattatcatcaccgtaatgcggatactcacgtcatgtccaaatgggtgaaactcatgttcctggtgtggatgccgtgcttgttgtgcatgagccgacctgatcgcgacgagccacccagcaatgggaacaagtcgtcggactccaagaacaagtcggcggcgtcgtccatccaccacctgcctgacctggagttccgggccaagtcgtccaggagcctactggctaacgtgctcgacctggacgacgacctacgctcgtccactcggagctacccgggagcccggagccaagtggcgccgatgcccgagcacgtgttggtcgaacagcagcagcaacagcacacgatgttgaccggcggcggaggcggagcggcgtcggcggtagccgcggcctcgtgtctggggccgcaccgcgagctgtcgctcatcctcaaggagctccgggtgatcacggacaagctgcgcaaggacgaggaggacgaggagaccaccaacgactggaagttcgcggccatggtggtcgaccgcatgtgcctgtacatattcacattcttcacggtggccgccaccatagcggtactcatgtccgcgccacatgtcatcgtcacgtgatcacgccaccgacgtcatcgaatccggttccggcttttatatatagattttgttatatattacaaaataaaatggttgcggcggagcagccatattatattgtatgacgaccacatctgaagtcgcatctgctagctcgcggaaaagtggcatgtagaaatcgatgtggtaccgttttaaatataatattatacgcatacgagataactataatattattaaaaattattaaattattattattaaataattattaaatactcctcctaatattatctaacattattataactattataattttttttcttgaaactacttcttttctgatgatggttaactaaagtatatatatatatctataaatatatatctatttggtttactatatgtaataataataatgatatatattaaactgcccctgctccttgaaacgaattaatgaacgcacaaaaacaatttgagtagatcgtacattattaatgtttaaatctgttccataaaatattaaaccggacacgttaaagtgtaaaacataatattatttacataaccgataatattaaaataatatatccgtgcgaataacatttcaaaaacaaacataatattttatacaagaaattaaaataggcatatttatactataatataacaaaccaattattgaatttactacaaagttggtacctatacctacgtttcggtgtacgatattgtcaaatttttaaaatatatttgttaattttttgcatattttcataagatcggaaaagttatagttgaatttgatgtagttaaactgccatgcgaatatattatattcgtttattcattttttttcatcttatttaaggaataagggcactacttatcaaaaaaaaaaaatttcctatttgacatcggaattctcataaaaatattatacacattctagaatcgataaatttttcattgtcacagcgtcgtaaaaccaacaacatgtcgtgacactattaagttttaaaatcagtaattattactatagcgatatattatcatattatataactggacatcgagatacaatatgaaatttctattactaatttccaaaaaaaattgattattctaaaatttttatttagcttagtataatagtgatattgtattttcacataaattcacttctttaaaattatctaacttctattgcatataatattgataatatttttaattattgatattgggttccttaatattattgatttaactgtgaatagtttgaaatacaaattcgattttcacaccaatgtaaacacattttgcctcgaaccgtcaacaatatatttagtttattatcattaatataccttcccacacagtgcaaaaacgcttatatcaaaatgcacaggtttcgcagctgctctgaaaataaaacaaagtacaagtacctaccttctccgagtggcttgattatcgacagtcattgtctttagatgcccacctataaagtataggttcaatatattactctaagtacttaactatttttggttttaataattaaaaattaaaaatgattttaaacatctttggctttgcaaattgcttacataacgtgtgttgctaatttaatttatttattttttaacggtgataataattattaataataatttttcattgggaaagtatttcactgatatagaaattgcaataaattataagtctgagatgatgaaaaaccataccattatagcgttttaaacgaatttaaaagtctacttgaacatttgtctgtagtaggaaatggattttccctcacgaaaaaacaatccaaatgtcaaactatgttttatttcaacttcggttcttaatacctatttatttttaaataatgtgctgataaaacgtatatgtagctataattaatattagtataattattagatcgtaaatattttactggaatacatgtgtcaataacttaatttattccataatctatctctataataattaataaaattactaaatattatacaattatttgtcatgacacccaatttttgtgtcttcttgctcaaattaatatatatatatatatatttatagacttgataagatcatattattagtttagctaaaacgatttgctttttattattaattatctgtgaaggaagaaataaatatagattagtcagacacaccttaatatctaatacgtaactaaataaatagattattatgcgatcggattatacaaataatcgtatttaaaattagtacaaaatcgatcacattatacattgtttatatattttttagacatgtattagagtacttaactgttttattttacgtgcacgttttgctgtgacaatcttaaaattttaacatcataacgcccaacggtaaattacaatagtaatactaataattattaaaattattatcaatatcattattattattattattattattattattattattattattattataaatttaactattaaaaatgtctaatgcattaaatgacaaaatcttgagtcgtcttccggttcaaataaatgtcataaaacaaaaacaaaaaaataaatgaaaaatatataggttataaaatacttaaatttcatttgatatatatacatatataaattatatactacattatacaatttagtagggatatcatagaatagcgatttggaattatttaattttcttcgatgatgtttcatgattgatcgttaacaaa

SEQ ID NO: 9 - DNA, 5524 bp, Acyrthosiphon pisum
  misc_feature 1..5524: note = alpha7 sub-unit of the nicotinic acetylcholine receptor
  source 1..5524: mol_type = genomic DNA
caccgccaccaccgccgtagctgtagccgtagccttcgtcgtcccgccacccgacgcaatacgaaacgcggtggtgcacgcgccgcccgggcagaaacacgggcgcgcgctacggcgagcaaatatagaggtagtatacgcgggcggtacggggcggggtctggaccgccgcggagaattgtgcgcgcgcgcgcgcgctcgtacggtcgcttgacggcagggcgaaagctagcggcacgcaccatcagcaccaccgacagcgacggcagcagcggcaggaccagcatttggagcagccagccggcggttggaacggcagtattgtggtagtggcgcatttggacgcacgtctgtgtgtgtgtgtgtgtgtgtgtgtgtttgtgtgtgtgtgtgtgtgtgcgtgtgtgtgtgtgagcgtgtgcgtgtggaagagagagagagtgagagagagagagagagataaagagagaaaaagctgcgcgtgcgtgcgtgggtgggtgtgtgtgtgacggcgcgcgccaaagacgttccgccgagtacatagggacaggttttcgtacacagtcgtaccgcggttaccgctgtcgcaataccgcaacgaaaacagctcatcaatataatattattattattatacgggcatcccataaaaatatattacatagtttccgtggacgagacgtcgtcgtcatcgcctgccgtcgtatcacactagctgccttcaccttaggaaccggctcccatccgttgaacagtcgcggacgctcgctgccgccgctctctgcagagccgctgcgaggtttttccgtctctatttttttccggcacgtgccacaccgaccacggacggcactgttccggtccaccactaccaccgttgaaatggacgcgacggtcggccaccgtctcgcggatacccatcggcgccacaacaccggcgacagacccggcgaccagcagctggtatacggccgtcagcgacgacaggatgacggacgtcagaaggagccactgatcgttgttggccacaacgcgtcctgttgttgataggagaccggacgtcgacgacgaggacgggtcgcgcgcgccaccgcaaccgccgtcgccgccgctgaatgggacatgggaatactcccgtatctgctcatcgtctacctgatcattgcagaaacgctatgcggacctcacgagaagcgattgctcaacgatttattagacccttacaatacactagagaggccagtagctaatgaatcggagccacttcaacttagtttcggccttatgctcatgcaaatcatagatgtggatgaaaaaaatcagctgttgataaccaacatgtggttaaaattggaatggaacgatgtcaacctcaggtggaaccatagtgactatggtggtgtaaaggatttgagaataccgccgcataagatatggaaaccagatatgctcatgtataatagcgctgacgaaggtttcgacggcacgtacgcaaccaacgtggtggtcaacagcaatggtagctgcgtattcataccaccgggtattttcaagagcacgtgcaagatcgatatcacgtggttccccttcgacgatcaaaaatgtgatatgaaatttggcagctggacatacgacggatttcagttggatcttcaactccaagatgatcatggaggagatatcagtggttttattactaacggtgaatgggacctgcttggcgttcccggaaagcgcaatgtgatctactacagttgctgccccgaaccgtacatagacataacgttttccatcctaattagaaggcgcaccttgtattactttttcaacctgatagtcccgtgcgtgttgattgcgtctatggccgtactaggatttactctaccgccggactgtggcgaaaagctatctctaggtgtaacgattctcctgtcgttgaccgtttttctaaacatggtggccgagacaatgccggctacttcggacgcagttcctcttttaggagtgacaatcctcctatcactgtccgtattctcactcatgatagccgatgccttaccacaaacatctgaagctatccccttactaggtacttatttcaactgtatcatgttcatggttgcatcgtcagtcgtgtccacaatcatgattctgaattatcatcaccgtaatgcggatactcacgtcatgtccaaatgggtgaaactcatgttcctggtgtggatgccgtgcttgttgtgcatgagccgacctgatcgcgacgagccacccagcaatgggaacaagtcgtcggactccaagaacaagtcggcggcgtcgtccatccaccacctgcctgacctggagttccgggccaagtcgtccaggagcctactggctaacgtgctcgacctggacgacgacctacgctcgtccactcggagctacccgggagcccggagccaagtggcgccgatgcccgagcacgtgttggtcgaacagcagcagcaacagcacacgatgttgaccggcggcggaggcggagcggcgtcggcggtagccgcggcctcgtgtctggggccgcaccgcgagctgtcgctcatcctcaaggagctccgggtgatcacggacaagctgcgcaaggacgaggaggacgaggagaccaccaacgactggaagttcgcggccatggtggtcgaccgcatgtgcctgtacatattcacattcttcacggtggccgccaccatagcggtactcatgtccgcgccacatgtcatcgtcacgtgatcacgccaccgacgtcatcgaatccggttccggcttttatatatagattttgttatatattacaaaataaaatggttgcggcggagcagccatattatattgtatgacgaccacatctgaagtcgcatctgctagctcgcggaaaagtggcatgtagaaatcgatgtggtaccgttttaaatataatattatacgcatacgagataactataatattattaaaaattattaaattattattattaaataattattaaatactcctcctaatattatctaacattattataactattataattttttttcttgaaactacttcttttctgatgatggttaactaaagtatatatatatatctataaatatatatctatttggtttactatatgtaataataataatgatatatattaaactgcccctgctccttgaaacgaattaatgaacgcacaaaaacaatttgagtagatcgtacattattaatgtttaaatctgttccataaaatattaaaccggacacgttaaagtgtaaaacataatattatttacataaccgataatattaaaataatatatccgtgcgaataacatttcaaaaacaaacataatattttatacaagaaattaaaataggcatatttatactataatataacaaaccaattattgaatttactacaaagttggtacctatacctacgtttcggtgtacgatattgtcaaatttttaaaatatatttgttaattttttgcatattttcataagatcggaaaagttatagttgaatttgatgtagttaaactgccatgcgaatatattatattcgtttattcattttttttcatcttatttaaggaataagggcactacttatcaaaaaaaaaaaatttcctatttgacatcggaattctcataaaaatattatacacattctagaatcgataaatttttcattgtcacagcgtcgtaaaaccaacaacatgtcgtgacactattaagttttaaaatcagtaattattactatagcgatatattatcatattatataactggacatcgagatacaatatgaaatttctattactaatttccaaaaaaaattgattattctaaaatttttatttagcttagtataatagtgatattgtattttcacataaattcacttctttaaaattatctaacttctattgcatataatattgataatatttttaattattgatattgggttccttaatattattgatttaactgtgaatagtttgaaatacaaattcgattttcacaccaatgtaaacacattttgcctcgaaccgtcaacaatatatttagtttattatcattaatataccttcccacacagtgcaaaaacgcttatatcaaaatgcacaggtttcgcagctgctctgaaaataaaacaaagtacaagtacctaccttctccgagtggcttgattatcgacagtcattgtctttagatgcccacctataaagtataggttcaatatattactctaagtacttaactatttttggttttaataattaaaaattaaaaatgattttaaacatctttggctttgcaaattgcttacataacgtgtgttgctaatttaatttatttattttttaacggtgataataattattaataataatttttcattgggaaagtatttcactgatatagaaattgcaataaattataagtctgagatgatgaaaaaccataccattatagcgttttaaacgaatttaaaagtctacttgaacatttgtctgtagtaggaaatggattttccctcacgaaaaaacaatccaaatgtcaaactatgttttatttcaacttcggttcttaatacctatttatttttaaataatgtgctgataaaacgtatatgtagctataattaatattagtataattattagatcgtaaatattttactggaatacatgtgtcaataacttaatttattccataatctatctctataataattaataaaattactaaatattatacaattatttgtcatgacacccaatttttgtgtcttcttgctcaaattaatatatatatatatatatttatagacttgataagatcatattattagtttagctaaaacgatttgctttttattattaattatctgtgaaggaagaaataaatatagattagtcagacacaccttaatatctaatacgtaactaaataaatagattattatgcgatcggattatacaaataatcgtatttaaaattagtacaaaatcgatcacattatacattgtttatatattttttagacatgtattagagtacttaactgttttattttacgtgcacgttttgctgtgacaatcttaaaattttaacatcataacgcccaacggtaaattacaatagtaatactaataattattaaaattattatcaatatcattattattattattattattattattattattattattattattataaatttaactattaaaaatgtctaatgcattaaatgacaaaatcttgagtcgtcttccggttcaaataaatgtcataaaacaaaaacaaaaaaataaatgaaaaatatataggttataaaatacttaaatttcatttgatatatatacatatataaattatatactacattatacaatttagtagggatatcatagaatagcgatttggaattatttaattttcttcgatgatgtttcatgattgatcgttaacaaa

SEQ ID NO: 10 - DNA, 5432 bp, Acyrthosiphon pisum
  misc_feature 1..5432: note = alpha7 sub-unit of the nicotinic acetylcholine receptor
  source 1..5432: mol_type = genomic DNA
ccgccaccaccgccgtagctgtagccgtagccttcgtcgtcccgccacccgacgcaatacgaaacgcggtggtgcacgcgccgcccgggcagaaacacgggcgcgcgctacggcgagcaaatatagaggtagtatacgcgggcggtacggggcggggtctggaccgccgcggagaattgtgcgcgcgcgcgcgcgctcgtacggtcgcttgacggcagggcgaaagctagcggcacgcaccatcagcaccaccgacagcgacggcagcagcggcaggaccagcatttggagcagccagccggcggttggaacggcagtattgtggtagtggcgcatttggacgcacgtctgtgtgtgtgtgtgtgtgtgtgtgtgtttgtgtgtgtgtgtgtgtgtgcgtgtgtgtgtgtgagcgtgtgcgtgtggaagagagagagagtgagagagagagagagagataaagagagaaaaagctgcgcgtgcgtgcgtgggtgggtgtgtgtgtgacggcgcgcgccaaagacgttccgccgagtacatagggacaggttttcgtacacagtcgtaccgcggttaccgctgtcgcaataccgcaacgaaaacagctcatcaatataatattattattattatacgggcatcccataaaaatatattacatagtttccgtggacgagacgtcgtcgtcatcgcctgccgtcgtatcacactagctgccttcaccttaggaaccggctcccatccgttgaacagtcgcggacgctcgctgccgccgctctctgcagagccgctgcgaggtttttccgtctctatttttttccggcacgtgccacaccgaccacggacggcactgttccggtccaccactaccaccgttgaaatggacgcgacggtcggccaccgtctcgcggatacccatcggcgccacaacaccggcgacagacccggcgaccagcagctggtatacggccgtcagcgacgacaggatgacggacgtcagaaggagccactgatcgttgttggccacaacgcgtcctgttgttgataggagaccggacgtcgacgacgaggacgggtcgcgcgcgccaccgcaaccgccgtcgccgccgctgaatgggacatgggaatactcccgtatctgctcatcgtctacctgatcattgcagaaacgctatgcggacctcacgagaagcgattgctcaacgatttattagacccttacaatacactagagaggccagtagctaatgaatcggagccacttcaacttagtttcggccttatgctcatgcaaatcatagatgtggatgaaaaaaatcagctgttgataaccaacatgtggttaaaattggaatggaacgatgtcaacctcaggtggaaccatagtgactatggtggtgtaaaggatttgagaataccgccgcataagatatggaaaccagatatgctcatgtataatagcgctgacgaaggtttcgacggcacgtacgcaaccaacgtggtggtcaacagcaatggtagctgcgtattcataccaccgggtattttcaagagcacgtgcaagatcgatatcacgtggttccccttcgacgatcaaaaatgtgatatgaaatttggcagctggacatacgacggatttcagttggatcttcaactccaagatgatcatggaggagatatcagtggttttattactaacggtgaatgggacctgcttggcgttcccggaaagcgcaatgtgatctactacagttgctgccccgaaccgtacatagacataacgttttccatcctaattagaaggcgcaccttgtattactttttcaacctgatagtcccgtgcgtgttgattgcgtctatggccgtactaggatttactctaccgccggactgtggcgaaaagctatctctaggagttacggttttgctatcgcttacattcttcctaaatatggtgtgcgaaacaatgccacctgcctctgagttaccactgataggtacttatttcaactgtatcatgttcatggttgcatcgtcagtcgtgtccacaatcatgattctgaattatcatcaccgtaatgcggatactcacgtcatgtccaaatgggtgaaactcatgttcctggtgtggatgccgtgcttgttgtgcatgagccgacctgatcgcgacgagccacccagcaatgggaacaagtcgtcggactccaagaacaagtcggcggcgtcgtccatccaccacctgcctgacctggagttccgggccaagtcgtccaggagcctactggctaacgtgctcgacctggacgacgacctacgctcgtccactcggagctacccgggagcccggagccaagtggcgccgatgcccgagcacgtgttggtcgaacagcagcagcaacagcacacgatgttgaccggcggcggaggcggagcggcgtcggcggtagccgcggcctcgtgtctggggccgcaccgcgagctgtcgctcatcctcaaggagctccgggtgatcacggacaagctgcgcaaggacgaggaggacgaggagaccaccaacgactggaagttcgcggccatggtggtcgaccgcatgtgcctgtacatattcacattcttcacggtggccgccaccatagcggtactcatgtccgcgccacatgtcatcgtcacgtgatcacgccaccgacgtcatcgaatccggttccggcttttatatatagattttgttatatattacaaaataaaatggttgcggcggagcagccatattatattgtatgacgaccacatctgaagtcgcatctgctagctcgcggaaaagtggcatgtagaaatcgatgtggtaccgttttaaatataatattatacgcatacgagataactataatattattaaaaattattaaattattattattaaataattattaaatactcctcctaatattatctaacattattataactattataattttttttcttgaaactacttcttttctgatgatggttaactaaagtatatatatatatctataaatatatatctatttggtttactatatgtaataataataatgatatatattaaactgcccctgctccttgaaacgaattaatgaacgcacaaaaacaatttgagtagatcgtacattattaatgtttaaatctgttccataaaatattaaaccggacacgttaaagtgtaaaacataatattatttacataaccgataatattaaaataatatatccgtgcgaataacatttcaaaaacaaacataatattttatacaagaaattaaaataggcatatttatactataatataacaaaccaattattgaatttactacaaagttggtacctatacctacgtttcggtgtacgatattgtcaaatttttaaaatatatttgttaattttttgcatattttcataagatcggaaaagttatagttgaatttgatgtagttaaactgccatgcgaatatattatattcgtttattcattttttttcatcttatttaaggaataagggcactacttatcaaaaaaaaaaaatttcctatttgacatcggaattctcataaaaatattatacacattctagaatcgataaatttttcattgtcacagcgtcgtaaaaccaacaacatgtcgtgacactattaagttttaaaatcagtaattattactatagcgatatattatcatattatataactggacatcgagatacaatatgaaatttctattactaatttccaaaaaaaattgattattctaaaatttttatttagcttagtataatagtgatattgtattttcacataaattcacttctttaaaattatctaacttctattgcatataatattgataatatttttaattattgatattgggttccttaatattattgatttaactgtgaatagtttgaaatacaaattcgattttcacaccaatgtaaacacattttgcctcgaaccgtcaacaatatatttagtttattatcattaatataccttcccacacagtgcaaaaacgcttatatcaaaatgcacaggtttcgcagctgctctgaaaataaaacaaagtacaagtacctaccttctccgagtggcttgattatcgacagtcattgtctttagatgcccacctataaagtataggttcaatatattactctaagtacttaactatttttggttttaataattaaaaattaaaaatgattttaaacatctttggctttgcaaattgcttacataacgtgtgttgctaatttaatttatttattttttaacggtgataataattattaataataatttttcattgggaaagtatttcactgatatagaaattgcaataaattataagtctgagatgatgaaaaaccataccattatagcgttttaaacgaatttaaaagtctacttgaacatttgtctgtagtaggaaatggattttccctcacgaaaaaacaatccaaatgtcaaactatgttttatttcaacttcggttcttaatacctatttatttttaaataatgtgctgataaaacgtatatgtagctataattaatattagtataattattagatcgtaaatattttactggaatacatgtgtcaataacttaatttattccataatctatctctataataattaataaaattactaaatattatacaattatttgtcatgacacccaatttttgtgtcttcttgctcaaattaatatatatatatatatatttatagacttgataagatcatattattagtttagctaaaacgatttgctttttattattaattatctgtgaaggaagaaataaatatagattagtcagacacaccttaatatctaatacgtaactaaataaatagattattatgcgatcggattatacaaataatcgtatttaaaattagtacaaaatcgatcacattatacattgtttatatattttttagacatgtattagagtacttaactgttttattttacgtgcacgttttgctgtgacaatcttaaaattttaacatcataacgcccaacggtaaattacaatagtaatactaataattattaaaattattatcaatatcattattattattattattattattattattattattattattattataaatttaactattaaaaatgtctaatgcattaaatgacaaaatcttgagtcgtcttccggttcaaataaatgtcataaaacaaaaacaaaaaaataaatgaaaaatatataggttataaaatacttaaatttcatttgatatatatacatatataaattatatactacattatacaatttagtagggatatcatagaatagcgatttggaattatttaattttcttcgatgatgtttcatgattgatcgttaacaaa

SEQ ID NO: 11 - DNA, 5634 bp, Acyrthosiphon pisum
  misc_feature 1..5634: note = alpha7 sub-unit of the nicotinic acetylcholine receptor
  source 1..5634: mol_type = genomic DNA
accaccgccaccaccgccgtagctgtagccgtagccttcgtcgtcccgccacccgacgcaatacgaaacgcggtggtgcacgcgccgcccgggcagaaacacgggcgcgcgctacggcgagcaaatatagaggtagtatacgcgggcggtacggggcggggtctggaccgccgcggagaattgtgcgcgcgcgcgcgcgctcgtacggtcgcttgacggcagggcgaaagctagcggcacgcaccatcagcaccaccgacagcgacggcagcagcggcaggaccagcatttggagcagccagccggcggttggaacggcagtattgtggtagtggcgcatttggacgcacgtctgtgtgtgtgtgtgtgtgtgtgtgtgtttgtgtgtgtgtgtgtgtgtgcgtgtgtgtgtgtgagcgtgtgcgtgtggaagagagagagagtgagagagagagagagagataaagagagaaaaagctgcgcgtgcgtgcgtgggtgggtgtgtgtgtgacggcgcgcgccaaagacgttccgccgagtacatagggacaggttttcgtacacagtcgtaccgcggttaccgctgtcgcaataccgcaacgaaaacagctcatcaatataatattattattattatacgggcatcccataaaaatatattacatagtttccgtggacgagacgtcgtcgtcatcgcctgccgtcgtatcacactagctgccttcaccttaggaaccggctcccatccgttgaacagtcgcggacgctcgctgccgccgctctctgcagagccgctgcgaggtttttccgtctctatttttttccggcacgtgccacaccgaccacggacggcactgttccggtccaccactaccaccgttgaaatggacgcgacggtcggccaccgtctcgcggatacccatcggcgccacaacaccggcgacagacccggcgaccagcagctggtatacggccgtcagcgacgacaggatgacggacgtcagaaggagccactgatcgttgttggccacaacgcgtcctgttgttgataggagaccggacgtcgacgacgaggacgggtcgcgcgcgccaccgcaaccgccgtcgccgccgctgaatgggacatgggaatactcccgtatctgctcatcgtctacctgatcattgcagaaacgctatgcggacctcacgagaagcgattgctcaacgatttattagacccttacaatacactagagaggccagtagctaatgaatcggagccacttcaacttagtttcggccttatgctcatgcaaatcatagatgtggatgaaaaaaatcagctgttgataaccaacatgtggttaaaattggaatggaacgatgtcaacctcaggtggaaccatagtgactatggtggtgtaaaggatttgagaataccgccgcataagatatggaaaccagatatgctcatgtataatagcgctgacgaaggtttcgacggcacgtacgcaaccaacgtggtggtcaacagcaatggtagctgcgtattcataccaccgggtattttcaagagcacgtgcaagatcgatatcacgtggttccccttcgacgatcaaaaatgtgatatgaaatttggcagctggacatacgacggatttcagttggatcttcaactccaagatgatcatggaggagatatcagtggttttattactaacggtgaatgggacctgcttggcgttcccggaaagcgcaatgtgatctactacagttgctgccccgaaccgtacatagacataacgttttccatcctaattagaaggcgcaccttgtattactttttcaacctgatagtcccgtgcgtgttgattgcgtctatggccgtactaggatttactctaccgccggactgtggcgaaaagctatctctagacatgagagccacgagaagcttgaaaaactactcttgttgtccgaatccgtacgtggatatcacttttaccatattcgttcgacgcaggacagtttattacatcgtcaacttgatattgccgagcgtaacgctggccgctatggccatgctcgggttcacgctgccctcagattgtggcgagaaaatgaccttaggagtgacaatcctcctatcactgtccgtattctcactcatgatagccgatgccttaccacaaacatctgaagctatccccttactaggtacttatttcaactgtatcatgttcatggttgcatcgtcagtcgtgtccacaatcatgattctgaattatcatcaccgtaatgcggatactcacgtcatgtccaaatgggtgaaactcatgttcctggtgtggatgccgtgcttgttgtgcatgagccgacctgatcgcgacgagccacccagcaatgggaacaagtcgtcggactccaagaacaagtcggcggcgtcgtccatccaccacctgcctgacctggagttccgggccaagtcgtccaggagcctactggctaacgtgctcgacctggacgacgacctacgctcgtccactcggagctacccgggagcccggagccaagtggcgccgatgcccgagcacgtgttggtcgaacagcagcagcaacagcacacgatgttgaccggcggcggaggcggagcggcgtcggcggtagccgcggcctcgtgtctggggccgcaccgcgagctgtcgctcatcctcaaggagctccgggtgatcacggacaagctgcgcaaggacgaggaggacgaggagaccaccaacgactggaagttcgcggccatggtggtcgaccgcatgtgcctgtacatattcacattcttcacggtggccgccaccatagcggtactcatgtccgcgccacatgtcatcgtcacgtgatcacgccaccgacgtcatcgaatccggttccggcttttatatatagattttgttatatattacaaaataaaatggttgcggcggagcagccatattatattgtatgacgaccacatctgaagtcgcatctgctagctcgcggaaaagtggcatgtagaaatcgatgtggtaccgttttaaatataatattatacgcatacgagataactataatattattaaaaattattaaattattattattaaataattattaaatactcctcctaatattatctaacattattataactattataattttttttcttgaaactacttcttttctgatgatggttaactaaagtatatatatatatctataaatatatatctatttggtttactatatgtaataataataatgatatatattaaactgcccctgctccttgaaacgaattaatgaacgcacaaaaacaatttgagtagatcgtacattattaatgtttaaatctgttccataaaatattaaaccggacacgttaaagtgtaaaacataatattatttacataaccgataatattaaaataatatatccgtgcgaataacatttcaaaaacaaacataatattttatacaagaaattaaaataggcatatttatactataatataacaaaccaattattgaatttactacaaagttggtacctatacctacgtttcggtgtacgatattgtcaaatttttaaaatatatttgttaattttttgcatattttcataagatcggaaaagttatagttgaatttgatgtagttaaactgccatgcgaatatattatattcgtttattcattttttttcatcttatttaaggaataagggcactacttatcaaaaaaaaaaaatttcctatttgacatcggaattctcataaaaatattatacacattctagaatcgataaatttttcattgtcacagcgtcgtaaaaccaacaacatgtcgtgacactattaagttttaaaatcagtaattattactatagcgatatattatcatattatataactggacatcgagatacaatatgaaatttctattactaatttccaaaaaaaattgattattctaaaatttttatttagcttagtataatagtgatattgtattttcacataaattcacttctttaaaattatctaacttctattgcatataatattgataatatttttaattattgatattgggttccttaatattattgatttaactgtgaatagtttgaaatacaaattcgattttcacaccaatgtaaacacattttgcctcgaaccgtcaacaatatatttagtttattatcattaatataccttcccacacagtgcaaaaacgcttatatcaaaatgcacaggtttcgcagctgctctgaaaataaaacaaagtacaagtacctaccttctccgagtggcttgattatcgacagtcattgtctttagatgcccacctataaagtataggttcaatatattactctaagtacttaactatttttggttttaataattaaaaattaaaaatgattttaaacatctttggctttgcaaattgcttacataacgtgtgttgctaatttaatttatttattttttaacggtgataataattattaataataatttttcattgggaaagtatttcactgatatagaaattgcaataaattataagtctgagatgatgaaaaaccataccattatagcgttttaaacgaatttaaaagtctacttgaacatttgtctgtagtaggaaatggattttccctcacgaaaaaacaatccaaatgtcaaactatgttttatttcaacttcggttcttaatacctatttatttttaaataatgtgctgataaaacgtatatgtagctataattaatattagtataattattagatcgtaaatattttactggaatacatgtgtcaataacttaatttattccataatctatctctataataattaataaaattactaaatattatacaattatttgtcatgacacccaatttttgtgtcttcttgctcaaattaatatatatatatatatatttatagacttgataagatcatattattagtttagctaaaacgatttgctttttattattaattatctgtgaaggaagaaataaatatagattagtcagacacaccttaatatctaatacgtaactaaataaatagattattatgcgatcggattatacaaataatcgtatttaaaattagtacaaaatcgatcacattatacattgtttatatattttttagacatgtattagagtacttaactgttttattttacgtgcacgttttgctgtgacaatcttaaaattttaacatcataacgcccaacggtaaattacaatagtaatactaataattattaaaattattatcaatatcattattattattattattattattattattattattattattattataaatttaactattaaaaatgtctaatgcattaaatgacaaaatcttgagtcgtcttccggttcaaataaatgtcataaaacaaaaacaaaaaaataaatgaaaaatatataggttataaaatacttaaatttcatttgatatatatacatatataaattatatactacattatacaatttagtagggatatcatagaatagcgatttggaattatttaattttcttcgatgatgtttcatgattgatcgttaacaaa

SEQ ID NO: 12 - DNA, 5439 bp, Acyrthosiphon pisum
  misc_feature 1..5439: note = alpha7 sub-unit of the nicotinic acetylcholine receptor
  source 1..5439: mol_type = genomic DNA
accaccgccaccaccgccgtagctgtagccgtagccttcgtcgtcccgccacccgacgcaatacgaaacgcggtggtgcacgcgccgcccgggcagaaacacgggcgcgcgctacggcgagcaaatatagaggtagtatacgcgggcggtacggggcggggtctggaccgccgcggagaattgtgcgcgcgcgcgcgcgctcgtacggtcgcttgacggcagggcgaaagctagcggcacgcaccatcagcaccaccgacagcgacggcagcagcggcaggaccagcatttggagcagccagccggcggttggaacggcagtattgtggtagtggcgcatttggacgcacgtctgtgtgtgtgtgtgtgtgtgtgtgtgtttgtgtgtgtgtgtgtgtgtgcgtgtgtgtgtgtgagcgtgtgcgtgtggaagagagagagagtgagagagagagagagagataaagagagaaaaagctgcgcgtgcgtgcgtgggtgggtgtgtgtgtgacggcgcgcgccaaagacgttccgccgagtacatagggacaggttttcgtacacagtcgtaccgcggttaccgctgtcgcaataccgcaacgaaaacagctcatcaatataatattattattattatacgggcatcccataaaaatatattacatagtttccgtggacgagacgtcgtcgtcatcgcctgccgtcgtatcacactagctgccttcaccttaggaaccggctcccatccgttgaacagtcgcggacgctcgctgccgccgctctctgcagagccgctgcgaggtttttccgtctctatttttttccggcacgtgccacaccgaccacggacggcactgttccggtccaccactaccaccgttgaaatggacgcgacggtcggccaccgtctcgcggatacccatcggcgccacaacaccggcgacagacccggcgaccagcagctggtatacggccgtcagcgacgacaggatgacggacgtcagaaggagccactgatcgttgttggccacaacgcgtcctgttgttgataggagaccggacgtcgacgacgaggacgggtcgcgcgcgccaccgcaaccgccgtcgccgccgctgaatgggacatgggaatactcccgtatctgctcatcgtctacctgatcattgcagaaacgctatgcggacctcacgagaagcgattgctcaacgatttattagacccttacaatacactagagaggccagtagctaatgaatcggagccacttcaacttagtttcggccttatgctcatgcaaatcatagatgtggatgaaaaaaatcagctgttgataaccaacatgtggttaaaattggaatggaacgatgtcaacctcaggtggaaccatagtgactatggtggtgtaaaggatttgagaataccgccgcataagatatggaaaccagatatgctcatgtataatagcgctgacgaaggtttcgacggcacgtacgcaaccaacgtggtggtcaacagcaatggtagctgcgtattcataccaccgggtattttcaagagcacgtgcaagatcgatatcacgtggttccccttcgacgatcaaaaatgtgatatgaaatttggcagctggacatacgacggatttcagttggatcttcaactccaagatgatcatggaggagatatcagtggttttattactaacggtgaatgggacctgcttgacatgagagccacgagaagcttgaaaaactactcttgttgtccgaatccgtacgtggatatcacttttaccatattcgttcgacgcaggacagtttattacatcgtcaacttgatattgccgagcgtaacgctggccgctatggccatgctcgggttcacgctgccctcagattgtggcgagaaaatgaccttaggagtgacaatcctcctatcactgtccgtattctcactcatgatagccgatgccttaccacaaacatctgaagctatccccttactaggtacttatttcaactgtatcatgttcatggttgcatcgtcagtcgtgtccacaatcatgattctgaattatcatcaccgtaatgcggatactcacgtcatgtccaaatgggtgaaactcatgttcctggtgtggatgccgtgcttgttgtgcatgagccgacctgatcgcgacgagccacccagcaatgggaacaagtcgtcggactccaagaacaagtcggcggcgtcgtccatccaccacctgcctgacctggagttccgggccaagtcgtccaggagcctactggctaacgtgctcgacctggacgacgacctacgctcgtccactcggagctacccgggagcccggagccaagtggcgccgatgcccgagcacgtgttggtcgaacagcagcagcaacagcacacgatgttgaccggcggcggaggcggagcggcgtcggcggtagccgcggcctcgtgtctggggccgcaccgcgagctgtcgctcatcctcaaggagctccgggtgatcacggacaagctgcgcaaggacgaggaggacgaggagaccaccaacgactggaagttcgcggccatggtggtcgaccgcatgtgcctgtacatattcacattcttcacggtggccgccaccatagcggtactcatgtccgcgccacatgtcatcgtcacgtgatcacgccaccgacgtcatcgaatccggttccggcttttatatatagattttgttatatattacaaaataaaatggttgcggcggagcagccatattatattgtatgacgaccacatctgaagtcgcatctgctagctcgcggaaaagtggcatgtagaaatcgatgtggtaccgttttaaatataatattatacgcatacgagataactataatattattaaaaattattaaattattattattaaataattattaaatactcctcctaatattatctaacattattataactattataattttttttcttgaaactacttcttttctgatgatggttaactaaagtatatatatatatctataaatatatatctatttggtttactatatgtaataataataatgatatatattaaactgcccctgctccttgaaacgaattaatgaacgcacaaaaacaatttgagtagatcgtacattattaatgtttaaatctgttccataaaatattaaaccggacacgttaaagtgtaaaacataatattatttacataaccgataatattaaaataatatatccgtgcgaataacatttcaaaaacaaacataatattttatacaagaaattaaaataggcatatttatactataatataacaaaccaattattgaatttactacaaagttggtacctatacctacgtttcggtgtacgatattgtcaaatttttaaaatatatttgttaattttttgcatattttcataagatcggaaaagttatagttgaatttgatgtagttaaactgccatgcgaatatattatattcgtttattcattttttttcatcttatttaaggaataagggcactacttatcaaaaaaaaaaaatttcctatttgacatcggaattctcataaaaatattatacacattctagaatcgataaatttttcattgtcacagcgtcgtaaaaccaacaacatgtcgtgacactattaagttttaaaatcagtaattattactatagcgatatattatcatattatataactggacatcgagatacaatatgaaatttctattactaatttccaaaaaaaattgattattctaaaatttttatttagcttagtataatagtgatattgtattttcacataaattcacttctttaaaattatctaacttctattgcatataatattgataatatttttaattattgatattgggttccttaatattattgatttaactgtgaatagtttgaaatacaaattcgattttcacaccaatgtaaacacattttgcctcgaaccgtcaacaatatatttagtttattatcattaatataccttcccacacagtgcaaaaacgcttatatcaaaatgcacaggtttcgcagctgctctgaaaataaaacaaagtacaagtacctaccttctccgagtggcttgattatcgacagtcattgtctttagatgcccacctataaagtataggttcaatatattactctaagtacttaactatttttggttttaataattaaaaattaaaaatgattttaaacatctttggctttgcaaattgcttacataacgtgtgttgctaatttaatttatttattttttaacggtgataataattattaataataatttttcattgggaaagtatttcactgatatagaaattgcaataaattataagtctgagatgatgaaaaaccataccattatagcgttttaaacgaatttaaaagtctacttgaacatttgtctgtagtaggaaatggattttccctcacgaaaaaacaatccaaatgtcaaactatgttttatttcaacttcggttcttaatacctatttatttttaaataatgtgctgataaaacgtatatgtagctataattaatattagtataattattagatcgtaaatattttactggaatacatgtgtcaataacttaatttattccataatctatctctataataattaataaaattactaaatattatacaattatttgtcatgacacccaatttttgtgtcttcttgctcaaattaatatatatatatatatatttatagacttgataagatcatattattagtttagctaaaacgatttgctttttattattaattatctgtgaaggaagaaataaatatagattagtcagacacaccttaatatctaatacgtaactaaataaatagattattatgcgatcggattatacaaataatcgtatttaaaattagtacaaaatcgatcacattatacattgtttatatattttttagacatgtattagagtacttaactgttttattttacgtgcacgttttgctgtgacaatcttaaaattttaacatcataacgcccaacggtaaattacaatagtaatactaataattattaaaattattatcaatatcattattattattattattattattattattattattattattattataaatttaactattaaaaatgtctaatgcattaaatgacaaaatcttgagtcgtcttccggttcaaataaatgtcataaaacaaaaacaaaaaaataaatgaaaaatatataggttataaaatacttaaatttcatttgatatatatacatatataaattatatactacattatacaatttagtagggatatcatagaatagcgatttggaattatttaattttcttcgatgatgtttcatgattgatcgttaacaaa

SEQ ID NO: 13 - DNA, 5436 bp, Acyrthosiphon pisum
  misc_feature 1..5436: note = alpha7 sub-unit of the nicotinic acetylcholine receptor
  source 1..5436: mol_type = genomic DNA
accgccaccaccgccgtagctgtagccgtagccttcgtcgtcccgccacccgacgcaatacgaaacgcggtggtgcacgcgccgcccgggcagaaacacgggcgcgcgctacggcgagcaaatatagaggtagtatacgcgggcggtacggggcggggtctggaccgccgcggagaattgtgcgcgcgcgcgcgcgctcgtacggtcgcttgacggcagggcgaaagctagcggcacgcaccatcagcaccaccgacagcgacggcagcagcggcaggaccagcatttggagcagccagccggcggttggaacggcagtattgtggtagtggcgcatttggacgcacgtctgtgtgtgtgtgtgtgtgtgtgtgtgtttgtgtgtgtgtgtgtgtgtgcgtgtgtgtgtgtgagcgtgtgcgtgtggaagagagagagagtgagagagagagagagagataaagagagaaaaagctgcgcgtgcgtgcgtgggtgggtgtgtgtgtgacggcgcgcgccaaagacgttccgccgagtacatagggacaggttttcgtacacagtcgtaccgcggttaccgctgtcgcaataccgcaacgaaaacagctcatcaatataatattattattattatacgggcatcccataaaaatatattacatagtttccgtggacgagacgtcgtcgtcatcgcctgccgtcgtatcacactagctgccttcaccttaggaaccggctcccatccgttgaacagtcgcggacgctcgctgccgccgctctctgcagagccgctgcgaggtttttccgtctctatttttttccggcacgtgccacaccgaccacggacggcactgttccggtccaccactaccaccgttgaaatggacgcgacggtcggccaccgtctcgcggatacccatcggcgccacaacaccggcgacagacccggcgaccagcagctggtatacggccgtcagcgacgacaggatgacggacgtcagaaggagccactgatcgttgttggccacaacgcgtcctgttgttgataggagaccggacgtcgacgacgaggacgggtcgcgcgcgccaccgcaaccgccgtcgccgccgctgaatgggacatgggaatactcccgtatctgctcatcgtctacctgatcattgcagaaacgctatgcggacctcacgagaagcgattgctcaacgatttattagacccttacaatacactagagaggccagtagctaatgaatcggagccacttcaacttagtttcggccttatgctcatgcaaatcatagatgtggatgaaaaaaatcagctgttgataaccaacatgtggttaaaattggaatggaacgatgtcaacctcaggtggaaccatagtgactatggtggtgtaaaggatttgagaataccgccgcataagatatggaaaccagatatgctcatgtataatagcgctgacgaaggtttcgacggcacgtacgcaaccaacgtggtggtcaacagcaatggtagctgcgtattcataccaccgggtattttcaagagcacgtgcaagatcgatatcacgtggttccccttcgacgatcaaaaatgtgatatgaaatttggcagctggacatacgacggatttcagttggatcttcaactccaagatgatcatggaggagatatcagtggttttattactaacggtgaatgggacctgcttggcgttcccggaaagcgcaatgtgatctactacagttgctgccccgaaccgtacatagacataacgttttccatcctaattagaaggcgcaccttgtattactttttcaacctgatagtcccgtgcgtgttgattgcgtctatggccgtactaggatttactctaccgccggactgtggcgaaaagctatctctaggagtgacaatcctcctatcactgtccgtattctcactcatgatagccgatgccttaccacaaacatctgaagctatccccttactaggtacttatttcaactgtatcatgttcatggttgcatcgtcagtcgtgtccacaatcatgattctgaattatcatcaccgtaatgcggatactcacgtcatgtccaaatgggtgaaactcatgttcctggtgtggatgccgtgcttgttgtgcatgagccgacctgatcgcgacgagccacccagcaatgggaacaagtcgtcggactccaagaacaagtcggcggcgtcgtccatccaccacctgcctgacctggagttccgggccaagtcgtccaggagcctactggctaacgtgctcgacctggacgacgacctacgctcgtccactcggagctacccgggagcccggagccaagtggcgccgatgcccgagcacgtgttggtcgaacagcagcagcaacagcacacgatgttgaccggcggcggaggcggagcggcgtcggcggtagccgcggcctcgtgtctggggccgcaccgcgagctgtcgctcatcctcaaggagctccgggtgatcacggacaagctgcgcaaggacgaggaggacgaggagaccaccaacgactggaagttcgcggccatggtggtcgaccgcatgtgcctgtacatattcacattcttcacggtggccgccaccatagcggtactcatgtccgcgccacatgtcatcgtcacgtgatcacgccaccgacgtcatcgaatccggttccggcttttatatatagattttgttatatattacaaaataaaatggttgcggcggagcagccatattatattgtatgacgaccacatctgaagtcgcatctgctagctcgcggaaaagtggcatgtagaaatcgatgtggtaccgttttaaatataatattatacgcatacgagataactataatattattaaaaattattaaattattattattaaataattattaaatactcctcctaatattatctaacattattataactattataattttttttcttgaaactacttcttttctgatgatggttaactaaagtatatatatatatctataaatatatatctatttggtttactatatgtaataataataatgatatatattaaactgcccctgctccttgaaacgaattaatgaacgcacaaaaacaatttgagtagatcgtacattattaatgtttaaatctgttccataaaatattaaaccggacacgttaaagtgtaaaacataatattatttacataaccgataatattaaaataatatatccgtgcgaataacatttcaaaaacaaacataatattttatacaagaaattaaaataggcatatttatactataatataacaaaccaattattgaatttactacaaagttggtacctatacctacgtttcggtgtacgatattgtcaaatttttaaaatatatttgttaattttttgcatattttcataagatcggaaaagttatagttgaatttgatgtagttaaactgccatgcgaatatattatattcgtttattcattttttttcatcttatttaaggaataagggcactacttatcaaaaaaaaaaaatttcctatttgacatcggaattctcataaaaatattatacacattctagaatcgataaatttttcattgtcacagcgtcgtaaaaccaacaacatgtcgtgacactattaagttttaaaatcagtaattattactatagcgatatattatcatattatataactggacatcgagatacaatatgaaatttctattactaatttccaaaaaaaattgattattctaaaatttttatttagcttagtataatagtgatattgtattttcacataaattcacttctttaaaattatctaacttctattgcatataatattgataatatttttaattattgatattgggttccttaatattattgatttaactgtgaatagtttgaaatacaaattcgattttcacaccaatgtaaacacattttgcctcgaaccgtcaacaatatatttagtttattatcattaatataccttcccacacagtgcaaaaacgcttatatcaaaatgcacaggtttcgcagctgctctgaaaataaaacaaagtacaagtacctaccttctccgagtggcttgattatcgacagtcattgtctttagatgcccacctataaagtataggttcaatatattactctaagtacttaactatttttggttttaataattaaaaattaaaaatgattttaaacatctttggctttgcaaattgcttacataacgtgtgttgctaatttaatttatttattttttaacggtgataataattattaataataatttttcattgggaaagtatttcactgatatagaaattgcaataaattataagtctgagatgatgaaaaaccataccattatagcgttttaaacgaatttaaaagtctacttgaacatttgtctgtagtaggaaatggattttccctcacgaaaaaacaatccaaatgtcaaactatgttttatttcaacttcggttcttaatacctatttatttttaaataatgtgctgataaaacgtatatgtagctataattaatattagtataattattagatcgtaaatattttactggaatacatgtgtcaataacttaatttattccataatctatctctataataattaataaaattactaaatattatacaattatttgtcatgacacccaatttttgtgtcttcttgctcaaattaatatatatatatatatatttatagacttgataagatcatattattagtttagctaaaacgatttgctttttattattaattatctgtgaaggaagaaataaatatagattagtcagacacaccttaatatctaatacgtaactaaataaatagattattatgcgatcggattatacaaataatcgtatttaaaattagtacaaaatcgatcacattatacattgtttatatattttttagacatgtattagagtacttaactgttttattttacgtgcacgttttgctgtgacaatcttaaaattttaacatcataacgcccaacggtaaattacaatagtaatactaataattattaaaattattatcaatatcattattattattattattattattattattattattattattattataaatttaactattaaaaatgtctaatgcattaaatgacaaaatcttgagtcgtcttccggttcaaataaatgtcataaaacaaaaacaaaaaaataaatgaaaaatatataggttataaaatacttaaatttcatttgatatatatacatatataaattatatactacattatacaatttagtagggatatcatagaatagcgatttggaattatttaattttcttcgatgatgtttcatgattgatcgttaacaaa

SEQ ID NO: 14 - DNA, 1722 bp, Acyrthosiphon pisum
  misc_feature 1..1722: note = alpha8 sub-unit of the nicotinic acetylcholine receptor
  source 1..1722: mol_type = genomic DNA
atgaatattttatttatcctcccgttactattgcctctcagctattgtgaattcgcgttaaacaatacaaaagtcgaaggaaacccttacaaaaaacatcttttcgatgaccttctgagcaactacaatcggctgatcagaccagtccaaaaccattcggagaacttgaccgtttggatgggtttaaaactcactcagctaaccgaaatgaatctcaaaagtcaagtgatgacaacgagtgtgtggttgatccagaaatggttcgactgcaatttgaaatgggatcccaaagactatggtggcttagacaagttatacgtgccatcggatcatatttggttgcccgacatagttttgtttaacaatgccgacgggaactacgaagtaaccttgatgacaaaagctgtgctcaagtataccggtgaagtattatggtcacctccggccatctacaaatcatactgcgagataaacgttctgtactttccgtttgacgaacagaactgttatatgatgttcggttcgtggacgtacaacggaaatcaggtagacttgaggcacatcgaccaatcgcagggcagaaaccgcgtggatgttggcattgatctaagcgaattctatctatccgtcgaatgggatttactcgaagtccctgcagtcaggaacgaagagttctattcttgttgctcagaatcgtatacggacataactttcaacataaagatgcgacgcaagactttgttctacactgttaatctgatcattccgtgcgtcggactgacattcatgacggtgttggtgttctatttgccgtctgactctggtgaaaaggtgacactgtgcagtaacattctagtgtcgttgactgtgttcttcttgctgctagccgagatcatccccccgacgtctctggccgttccactcctcggcaagtacttgttgtttaccatggttctagtgtccatgtcgatcggcatgaccgtcgtcgttttaaacatccatttccggtcaccatcaacccacaccctgtcaccttgggtgagaaccgtgtttctgcacatgatgccacagatactgatgatgcgccgaccgccgtattcgctgatgaccaacgacggattccaaagctcacccggttacttcaacgaactggactacagtcgagacagcataagcgatagcggtggaaccgatcagaaacctccatctttgacccagagacactggtccttcaaaaaaattcaatctgtacagcctcacaattatccggagaatgtaatgccaaagacaatgtccccagatctactcaaggctttacaaggggtctgttatattgctcaacatataaaagacgcagacaaagataaagaggtgatcgaggactggaagtacgtgtcgatggtgttcgaccggttcttcctgtgggtgttcactctagcctgtatagtgggaacatgcgccataatattccaggcaccctcgctgtacgaccaacggcaacccatcgacttccagctgtccagcataccccgccgccggagcaacctggcactacctcatgaaatcgacttttacgagcgcacctatggtgccaactagagtatctaactattgcattttatagatatatccgttgtaatcccactgcaggctcattttaatatgtacataattaaatatatatatatacttataaacctacgtagccatgtagataacgaa

SEQ ID NO: 15 - DNA, 1424 bp, Acyrthosiphon pisum
  misc_feature 1..1424: note = alpha9 sub-unit of the nicotinic acetylcholine receptor
  source 1..1424: mol_type = genomic DNA
aaatcattttgcattcgactacgctccgcgaatattattttccttagtagtttccagtgtcgatatcgccaccggacacgatggacggtccacgactgtcaggattgttcctgataattttaaccacatactgtgcgaccggcgacgcaagtaattcaaaactaccagacgaaaacatcacggctggacatgagctgagaaaacaactgttgaagaattacgataaagtagtggtgcccgctacaagcaaaaaacctgttcatatgaaagttaacgctgtatttaacagcgtagaaatgagttacgaaacgtctcatatgatcatttacgccagcttaaaagtggcgtggattgacgatcgactgacatgggacccgaacgattacgataaaatcaatttcatagctgttgaacacaacgaaatttggcgtccagacatcatggcctacaacaacattgaggccaacaccgtagtgcacggaaacacacagagcatcgtgtacaacaccggagacgtcttgtgggtgcaacccgtgcagtacaggatatactgccaagccgacatgacctattggccgtatgacacgcaaaccggactgttgatagtcgggtcgtgggtttacagcacgaacgcaataaacatcacagacgccgcgcacgagattttcctcacccctcacacggaatggcagatattacgaatttccagcgataaaataacgaaattctattcgtgttgcccgaacgaaccatacgtcagtgtcgagtacaacatcaccatcaatcgcaagtccaaccaatatcatccggtggttttcgtaccagccttatgtacagcactattgaatttactggtgtttgggctttcactagatgacaacagatacaaacttacaataagcctattcgacgcattgattgttgtcgtcattctacaggtgctgtactcgaaaatacctctgatactatccacagttccattaattgctatatactacacgtacagcctgggactgatagttttgacggctataatatcagtggtgataaaaaatatgacaataacacggaaaccactttcaaaaccaatttcaaacatcgttgaatccaaatttatagaattcctagcaatgggagtggacggaccatcgtcgagcaaccaaactctaaacgaagacaaagaaatagatttacaatatatagagtataacaaaagacagacattcgctaaaataattgataaaatatgttttattatattttcaattatttatggtattcttctatcacgctttgtgttttaattgatgaatataatatgatatttttaagttatttatcatttacaatatcatataatttggattaattaatgtacaataattattaaaatttggcctcgtatttatatgcttgcaaatgta

SEQ ID NO: 16 - DNA, 1081 bp, Acyrthosiphon pisum
  misc_feature 1..1081: note = alpha10 sub-unit of the nicotinic acetylcholine receptor
  source 1..1081: mol_type = genomic DNA
tcctgtactcaagtggacagaatgttataatcacggacgttcgtaactaaattgtccagtattccacgcgtacgcacaatacgttcacacaaccggtcaagttgttttttttcgatagatcgttgttgctcgtggtttttgaattctctcaacaatgttgtgcactcatcatattggattattcataattattatagctacacaagctacacacctgtgggctttcttgagaacagtccagatatataatcacaattcaacagtagaacatatcttgagaaaaaatatttttaagaactatgacaagatagtgaaaccttacgccccccaggataaaaactccttagaagtgagcatgaaaatgttggtaaaaagtgcagagttgaattatgaaaaatctcaaatgatactcagcacatggttcatagcgaactggatcgacgataggttgacttggaatccgagcgaatatgaacacctgaattctataatagtcgaccaccgtgagatctggcacccggacatcatggcgttcaacaacgtcgacgccaacatggaagacgatcggacacacgtcaacagcgtgttgaacagaagcggtggggtgatatgggttgaaccggtgcagtacattatacattgcaagacggacacaacaaactggcctcacgacacgcagaccggagtcttgaaactcgggtcgtggttgtacttgggcagagacctgaacctgaccatcgacgaaggagaagaagtagaaatggctagctcacactcggaatgggacatcactaacgtttccaaggagagaaacatcaggtactatgactgctgtcccgacgaacaatatatcgacatcgaatacaatatcaccatcaggcggaaggtgcattcctataagtcagtcctctatatcccagctctatgcagtatttcgttcaatttactaacattttggctctcgtactccgacaatcaaagtaaattgtttgtgaacctgttcaaacgcactgttcgtcactttggccattatggtcgtgtacacaaaagtaccgattgtcacttccactgttccggttga

SEQ ID NO: 17 - DNA, 4051 bp, Acyrthosiphon pisum
  misc_feature 1..4051: note = beta1 sub-unit of the nicotinic acetylcholine receptor
  source 1..4051: mol_type = genomic DNA
cgcgctatcgtttcgcgctatactacgcacccacacactcgcgcgtacacacaacacacacatagtgccgcgctcgcgcacaacgtaagcggtaatacacacacccaacacacacacatgcacacgcgcgcgcacacgttgagggtccgtagtacacgcagcagcagtattgtgtgtggtgtgcgtgacgttcgttttctgcagaaacactgtgcgaattttttttagttttcgttttgcgcgtccgatatttgttttcggttttttttgtaaaccgttcctgtggtcatcgtcatcagtgtaatctcttgtggattttaccgtcgttgtagtcgttttcgaaattggtttctcacggtctaccgtcggccggtggtgcaggtggtctcccctgtcatgcccgcgatccgcgtcgccgtcgtcggaatcggtgcacgacgaccgccgccgttgccggaccgctgtccggaacacggccgcttctgatccactggtttcgtagttgtctcgagatgacggtaacggccacggcctctgctgttgctgccggtgctgcggtgattaccacaaccatcatcaccaccgccatcaccgccactatcacgtcgacaggacgtgacgcgtcgcggcggtaagaagagcgcacgcggtcttttccggcccactcgtccaccccctcactacccctaaccaaccgctggtaaccgtgtttttgcacggcgacggtgctggcagcagcggcagcaggcgcgacgtattgttttggacgatatgaagtgctgcgttacgcgggacgcgtgaacgaaaatgcaacgtgacaaggccataatagtctttttcactacagctattaaaattactgcgaggacgacggagacgattgcgaccaccgtacaagtgccaaaggccgcagttaaaacatgaacactcccgttggactgctgatggccgtgttcttcgtctgttcacagttcatccgaggttgttggtgttcagaagacgaggagcgtctggttagagatcttttcaggggctacaataaactaatcaggccagtccagaacatgaccgaaaaagtcaatgtacagtttggtttagcattcgttcaactcatcaacgtgaatgaaaagagtcaaataatgaaatcaaacgtttggttgagacttgtatggagggactatcaattacaatgggacgaagcagactacggtggtatacaggtgctcaggttaccaccggataaagtgtggaagccggatatagtgttgttcaacaatgctgatggaaattatgaagttcgttacaaatcgaacgtgctcattcgaccaaatggagaacttctatggataccaccggcaatttaccagagctcctgcaccatagacgttacatattttccttttgatcagcaaacttgcataatgaaatttggctcttggacatttaatggcgatcaagtatcgttagcgttatacaatgacaaacagttcgtcgacctgtctgactactggaaatccggaacttgggatattatagaggtacctgcgtatctgaacgtataccaggagtcgcctacccagacggacattacattctacatagtgattcgaaggaaaactctgttctacacagtgaatttgatactgcccacggtcctcatatctttcctgtgcgtgctagtgttctacttgccagccgaagccggtgaaaaggtcacgctgggcatcagcattttactgtcactagtcgtcttcctattgctcgtctccaaaatactgccacccacttctttagtactgccgctcatcgccaaatatttgctattcacgttcatcatgaacaccgtcagcatattggtcactgttatcataatcaactggaattttcgagggcctcgtacccaccgtatgccaccttggataaggactgtgttcctatactaccttccggcatgcatgttcatgaaacggccgaagaaaacgcgactgaggtggatgatggaaatgccgggtatgagtggtccgccacatccgcaccacacgtcaccgtcggacctacccgcgcccgcgcccccgagttccgcgacgccgtccaaacacaagatggaggcaatggagctggccgacctgcatcatcccaactgcaagatcaaccgaaaggcaagcgcggagcggagggagagcgagagctccgactcgctaatcttatcaccggaggcatccaaggccaccgaagcagtggagttcatcgccgaacacttgaggaacgaggatcaatacatacagataagagaagactggaagtacgtagcgatggttattgatcggctgcagttgtatctgttcttctttgtcaccaccgccggcacccttggcatactaatggatgcccctcatatattcgagactgtcgaccaggacaaaatcatcgaaatctacggcggaaaataaacatttaagaaataaaaaagatttatagatgaaaatgctcaagttagaactaatttgcttcagaggagttatttaaaataataataataattaaaataataattttattattttaaaattaatgaacaaaaaaaaaacagtactgtataaccatcgtgttgttattattaatataaaaatacaacttagttagtttaagtgccaaaaaaaaagaaatgttatttattactattgtgtaaaataattaatgatgtttaagaaaaaaaaacaaataatattactaatatttttagattagtgttaaagttggaatgaacagctcgcgaaaatgtctcgaacgtccgtacgtatactgtttgtattgcaaaaatatcctaaggggttttgaatgtgctcaaatcgttcattttccaagcacaacacacaggggggtgatttatcacagattcaaaccaaatttattttgtcctttagaatatctcgtatttccgctgtgtcccttccaataacgcaccggtatgctgtgaaccgctcaccctgtgtaactccaatgtgccaatatcgttgacgtgacagatgataaactaaattttttttgctcaaactcgtcgcgtataatatgtaaacaaaataaatggcccaaaattattattttataggtttttctaatgacaataattaaaaagaaaagccgaaacatattattactaccgcaatgtttgtttataattatagtttaatttgtttatcataatatttcctcatctaaatatattaaaatcttcgctcaaatatttttcgcttatgctgtactatagacgatgacgtccgggacatattcgtcaaattttgaaccaacgtttggtgtacataactattattgttatatttttttagtttttttatttctcagtttagtagaagtcattccgtgcccggtttgtctagaacactcaaattatgaacattatataagaataactttgcgcggtctactattatattataataatgataatattatacgtgtttgaatgtgcccaacgattgtctgggtgatggagctaaccgggctaatattactgtccataggttaaatccaacgctggtttcaaaatggtcgtatatacaaattaatcgttagaaaccgtttgcaactttctagcataacctatgttataatcaaatttatattaaatactaatatctaatcatttattgttattattattattattattattattattattattattattattattattatagtgtgcactaacggcatacggctcacgttagttgtatactagaatttagtttattttattatttcattaaaaaaaaaaacaaattaatttgtttttatttttgttttttgtgtagtgtgtgttgcaaaattttggtgtacgtacaattttgttactgcgttttaatattataataatgttacaacccttgaaaaggggaattttttttaagtattgtataaaaataataaatatagttaagtcatggaagatatacaacacagctgttacaatcatccgtgtacacaataataatttaattacaaaaaaaaaaaattaagaaattca

SEQ ID NO: 18 - DNA, 1828 bp, Acyrthosiphon pisum
  misc_feature 1..1828: note = beta2 sub-unit of the nicotinic acetylcholine receptor
  source 1..1828: mol_type = genomic DNA
tatcagcactcagttgtgtcaaaaccgttttttaaacaacacatagcattctgactacagcgagaggattaacaactatgtataccaatgtgattacctacctataattgcattactattttttcagtttgaaatagttaacaatataatacagatatttgtctatatattatacgccacctaatattcgttttttgatttacgttaccatacttgtattcaaaacatttccaaataataattatgtttaaaagttttttaaaaaaagttttgtgtttgattatcattatcaaatttgcgaatatagatgctgcaataacagacaattatgggaaacaacattggaacgacacgtggacggataagctgagaagagatttattgaggttctacgatagacactcgagaccggttatcagtgatcatctgaccaatgtcacggtcaatactgttttaaaatatgtcgatctggacttccaaaattcaattatgaccattaacgcctgggtatcgatgacttggcaagatgaaaaacttctgtggaatgtatcagattacggagacacaaaagttgtaaacatggcttatgatgaaatatggatgcccgatattgtggcctacaacagtgctgacgggcatcatgacgagacttatgggaagattaactgtgtagtccgttcggatggaatggttcattggataccaccagccaagttccgagtcttttgcgatgtagacttgaccaattggccatttggagagcacacctgttcgttgagactcggctcttggacgtacgacggcaattccgttagcatgcaaatcaatcagaaaataaacactgccgacatgataaacttggaagaaggttgtaaatgggacatcacaaaggtaacgcaaagacgaagagtcgagagctactcttgcaattccaacgacatatacgaagacattcactacagcatcaccgtgaagaggaaaataccggcgtaccacagcatagttgtcataccagcgatagtcgtcgtgttcctgacgctatccgtcttttggctacctccggagtcgacggagaagtttatgttgagtggcgttactctggtcatagtatgcatactgttaatgatgatctccggcaatgtgcccatcatgagcgaccgtatcccgttaatagtaatgttttactcaaactcgctgggcatggtcactctatcgatggtattctcagtgttgttacacaatctagctaaatacaagcaacccgaccataaagtacccaagtatatcactcagctgttgaacacaaaattcggaaccgtcatgagatttacatcaatgaagaacgatgacgaggacaagatattggacgaatcaaatgatgataaatcacaatatcctcaactgaaagaaagtcaatggtattatctggctctgattttagatagaacgatgtttattgtttacttatttttgttcattgctatggtgatttattttctgaagtgaataatatgagagtaatatttcacaaaagatcaatgtttgtgtatctatctactagtttctattaaggaacattttttttttatattattcgtatcgatttttgctcttttataactcttatatttttataaatttataaacaaaggtattttttaattatatttatatctaatcctattattaaaaaagtatagttacttatttattgattataatgaatatttttggctttaattattaaatataggtactttaattttatattttatattaatgtatatgagtatctatatgactaatatctataatttgatataagttt

SEQ ID NO: 19 - DNA, 2502 bp, Periplaneta americana
  misc_feature 1..2502: note = alpha1 sub-unit of the nicotinic acetylcholine receptor
  source 1..2502: mol_type = genomic DNA
catggggaaaactagttctgaatctaccctgactctgtcaataacatatacatacaacagacgggtgccacaattgtgtcttcctcaactatgaggaaatcgtgagataaaattcatcggggctctagacgcattcggtctggaatatcaagccgcagctctcgcttgtgccggtacttgtaaagtgtttgtttcattttaatttcgggggtgtccagtaaaacgtctcgggtgatattgagtggttgttgctgatagcggggatgagaagctcacagctggcggccggggacgggacacgaagccgggagatctcccggtcccacggcgctcaactccagcttgtggtgaagcagtgacctcttccagtggaaggaaggcgatgctctcagcggtgatctcaccgggtggactaggcctaagatggggagcgtgtttctggcgctccttctggcggtgatcgggggaaccgtggccaacccggaagcgaagcggctgtacgacgacctgctgagcaactacaaccggctcatccggcccgtgggcaacaactcggaccgcctcaccgtcaagatgggactgagactctcgcagctcattgatgtcaatctgaagaatcagatcatgacaacgaatgtgtgggtagaacaggaatggaacgactacaagctgaaatggaatccggatgactacggcggtgtggacacattgcacgtcccgtcagaacacatctggctgcccgacatcgtgttgtacaacaacgccgacggcaactacgaggtgacgataatgactaaagcgatcctgcaccacacggggaaggtagtgtggaagccgccggccatctacaagtccttctgcgagatcgatgtcgagtacttcccgttcgacgagcagacgtgcttcatgaagttcggatcctggacgtatgacggctatctggtggaccttcgccatatcgcccagtcgccggactcggacacgatagacgtcggcatcgacctgcaggactactacctgtcggtggagtgggacatcatgagagtgccggcggttcgcaacgagaagttctacagctgctgcgaggagccttaccccgacatcatcttcaacatcactctgcgaagaaagacacttttctacaccgtgaacctcatcatcccgtgcgtggggatctcgttcctctccgtgctcgtgttctacctgccgtccgactcgggcgagaaggtgtccctctgcatctccatcctcctgtccctcaccgtgttcttcctgctgctcgccgaaatcattccgccgacctcgctcacagtccctcttttgggcaagtacctcctcttcaccatggttctagtcaccctctcggtagtggtaaccattgctgtgctcaacgtgaattttcggtcgccggtgacacatcggatgaggccttgggtgcaccggttgtttatccagttactcccgaaggtgttacttatagaaagaccgaagtccgaggactctgtggaggacgaggaggacgaactcaagcctcccgagggggtcctcacgggagtgttcgacgtatcttctgatatggacaagtacatgacgtacggaggaaagaggttcagcgcggactacgatatccccgctctacctccgtcgcgtttcgatacgggaggggggccttgcttcggggaaccaccactgcctctaccgggagccgatgatgacctcttcagtcctggcactggctgcgtggctggagcggcatcggcggtttgcggagcccacgatcaaagcccgacgttcgataaaccgtcgcacgagatggagaaaaccatcgaggacgccaggttcattgctcaacatgttaagaataaagacaaattcgaaaacgtagaagaggactggaagtacgtggccatggttctggaccgcctcttcctctggatcttcaccgtggcctgcgtcctaggaaccgcgctcatcatccttcaggcgccgtcgctgtacgacacgacgagacccatcgacatccagtactccaaggtggccaagaaaaagatgcgcatgatgatgatgggtccggaggaggactgaagattacgaatgttgcaggggatagtgccagtaggtgcggccatgcacattacatttttattaaatcagaggtgtttccactccgaaaattttaagtaaatacatttttgtttggatatgagagttaaaatgagtacacaatttggagaactcgtctcctattacgtatacagtgttttcattaagtgtggaatattattcataatgtaataaattttaattttacaaaacaaaaattatacaaaagctgttggagataaaccttacagtatgaaactagtgttagaaggaagttattcagccataaatacatgatagtaaactttattaagaccgacatattacgaatcgccattaaattttatgtatgaatccttagaaattttgcccatttacccctccaccaaaaaaaaaaaa

SEQ ID NO: 20 - DNA, 2657 bp, Periplaneta americana
  misc_feature 1..2657: note = alpha2 sub-unit of the nicotinic acetylcholine receptor
  source 1..2657: mol_type = genomic DNA
catgggggactgcccgtttctcggtatcgcgcagcgcgccgttggttgatcgtccgtccggggagagagcagagcgatccgctgcaaaacatacagtgcataacacgtggaaaacgacgcctacattgtgcgacttacacaagtatcaatgacaactttcagtcataaacaacacaacgtctgtaacagcaacatcaggccagcatttgtatacaaacacgctccacggttaaggtaaccgcgtttccctgttttgtagtgatgcgacgcagagatagatcaactactcgtgtgcaccacagatttaagaactcgagaagaaaatgacatcgtgttcatagaatggacggcgaccctccgtgttgtttagctcttaacgtttcttcctccgaaagataccacccgggtcgtgatcgcatgtgtattatggtgttattagttctactgggatgtataacaactccatctcatgccaaccctgacgccaaacgcctctatgatgatctgctcagcaattacaatcgattgattcgaccagtcagcaataatactgatactgtgctagtcaaattgggccttcgtttgtcgcagctcatcgatttgaacttgaaagatcaaatcctaactacaaacgtctggctggaacatgagtggcaagatcacaagttccgatgggacccgcaggagtatggcggcgtcacagagctgtacgtgccgtcggaacacatctggctgcccgacatcgtactctacaacaacgcggacggggagtacgtggtcaccacgatgacgaaggcagtgctgcaccacaccggcaaggtgctgtggacccctccagccatcttcaagtcgtcttgtgaaatcgacgtgcggtatttccccttcgaccagcaaacctgcttcatgaaattcggatcttggacttacgatggatttcagatcgacctgaagcatatcaaccagaagttcgacgacaacaaagtggaggtgggcatagacctgcgggagtactaccccagcgtggagtgggacatcctgggagtgccggcggagagacacgagaaatactacccctgctgtgctgagccctacccggatatcttcttcaacattacgctgcggcggaagacgctgttctacaccgtgaacctcatcgtgccgtgcgtcggcatctcctacctctcggtgctcgtcttctacctgccggccgactccggggagaagatcgccctctgcatctcgatcctgctgtcgcagacgatgttcttcctgctcatctcggaaatcattccttcgacgtcactcgcactgccattgctgggcaagtacctgctcttcactatgatcttggtaggcctctccgtagtcataaccatcatggttctcaacgtccactatcgcaagcccagcacacacaagatggcgccctgggtcaggaaagtcttcataagaaggctgcccaagttgttgttgatgaaggtgccggaacagcttttggccgacttggccgccaataaaagccgcttgcgggccgctaaaaaatcgaagctcaacgccgcagcagcggcagctgcggcagcagccgcctcatcgtcgtcggtggcgtcgtcgccagactcgctccggcaccatcatcaccaaaggcctggcgggtgcaacggactgcattcctcagcaagtggtgccaccaacaggtttggagggctcatcgcaggcttcaacggactgccgtctgtggtgggactggatgggagcctcagcgacgtcgggaatcgcaagaagtaccccttcgaactggagaaggcgatccacaacgtgatgttcatccagcaccacatgcagcgacaggatgaattcgacgcggaagaccaggactggggcttcgtggccatggttctggatcgtttattcctctggatcttcacgatcgcatcgatcgttggaacgttcgccatcttgtgcgaagcgccggccctctacgacgacaccaaaccaatcgacatggaactgtcgtcagtggcccggcaacagttcctacccgacttggaattctagcagtaaattccctcgccacttgtgatagtggcgaatggacgatatcgcgtgtatatcaaagtgaagctgaaaatgattccacagatttccagttcaacatgtcttaacggcagtgggtgctcctcctcttgtcataaatacattaatgatggtggaattggaaaatggtgcagatgaggaaagaagaacattccagctccatgtgtgttgtgtcaaaaggattttctgtgttgacagtgcgtattcaagcacttctacaaaatgcataaagtgaaaaacacaagacaattagagaagctgaaactatgaatgttcacaaaatttatgctggacgtatagaagagaaaataataggccagaaaatatgaagaggctgttgtcaaaaaaggagctgaagatttctgaaaggaggaagaaaaaacataaatagactggaatcagctgacttcaccgggttttgagggttcctgtatcaatcaaacgtttaaacatttacttatatgcgttttaatgatatgaatactagaaattgcttattatctcttgttaactaaatgttatgtacgattataataaatatttaattattgaaaaaaaaaaaa

SEQ ID NO: 21 - DNA, 2068 bp, Periplaneta americana
  misc_feature 1..2068: note = alpha3 sub-unit of the nicotinic acetylcholine receptor
  source 1..2068: mol_type = genomic DNA
gccaagcagaagatgaggaaccttttggctctcatgtggatcctgttcatatcaggttgctcgggcaaccccgacgccaagcgcctgtacgacgacctcctcagcaactacaacaaactggtgcgtcccgtcgtcaacgtgagcgacgcactgacagtcaagatcaagctgaagctgtcgcaacttatagatgtgaacctcaagaatcaaatcatgactaccaacctctgggtggaacagtcatggtatgactacaaactacaatgggacccaaaggaatatggaggagtagaaatgttacatgtgccatcggaccacatatggagacctgacatcgtgctctataataatgctgacggtaattttgaagttaccttggcaacgaaggcaacgttgaactacactggccgcgtggaatggaagccgccagctatctataaatcgtcgtgcgaaatcgacgtcgagtatttcccgttcgacgagcagacttgcgtcatgaagttcggatcctggacttacgacggctttcaggtggatctccgtcacatcgacgaggtgaaggattcaaacatcgtagagatcggcgtggatctctcagagttctacacctctgttgagtgggacattctagaagtgccggcagttcgaaatgagaagtactatacgtgctgcgacgagccgtatctggacatcacattcaacattacaatgagacgcaagactcttttctacacggtgaacctaataattccttgtatgggcatctcgtttctcaccgtactggttttctacctgccgtctgacagcggtgagaaggtgagtttgtccatctcaatcttgctgtcactgactgtgttcttcctactgctcgcggagatcattccccccacatcattggtcgtgcccctgcttggaaaatttgtcctcttcaccatgatcctcgacaccttcagcatttgtgttacagttgtagtgctcaatgtccacttccgttcaccacagacacacacgatggctccgtgggtacgtcgtgtattcattcatatacttcctcgtcttcttgtaatgagacgcccgcagtaccaaatcgataaacgcagtctagcgggttcatctgcgagaataatggtgcgcacgtgtaatgggcttgaactgcgagatcccacgctgctggctgaatcatccgcttccgaactcgtggactcgacgcataccacagaactgttcccaacattagactctcacgacgagtttactcccagggaactggaagcaacaggtctgggatcagggtgtcggatccatggcacaccaccactacctccattatgccaagaagatggattcctggcaccaggtccacctgaagaggcctctagtccattatgcagacatttgcaccattggcacaactgcccagagctacacaaagctatcgatggggtcaagttcattgctgatcacaccaaaagggaagaagactccaccagggtgaaggaggactggaagtatgtagcgatggtgttagatcgattgttcttatggatcttcacgctagccgtcctggtgggaacagctggtatcatcctacaagcaccaacgctctatgatgatcgtattcccatcgatgttcgcctatctgagatagcttccacgacagccaaaccccacatagccactggcctctgaacattattgtatttgcgctctgaacgcacaaatatcacgaagaagcccattggggtgtggccctgatgtagagcatagcgagtcgcaagcaatggtgtcgctaaatcaaaatactggacacaatggtattcccatctttgtcatttttaaagcttttttattgaggggtggagtttgtttgtttctgttccaacatggctgtcaggcgccacatgaagaatggcctaaatatgcacaggaggatattatggggtattgtggtcactcataggaatttgcttgcctttttatttgatgactgaacaaatgttaaggtaaatgaatatggacagaaagtgcttctcttatatttcgattccgtactaccaaaaaaaaaaaa

SEQ ID NO: 22 - DNA, 2200 bp, Periplaneta americana
  misc_feature 1..2200: note = alpha4 sub-unit of the nicotinic acetylcholine receptor
  source 1..2200: mol_type = genomic DNA
agtgcgagggatgacgtcgcggtaacgagatggtgtgtgccggggatgcgctgagggcctgggtgctatcggcgctggtggtgcacggggcagtggcgggcaaccccgacgccaagcgcctgtacgacgacctcctcagcaactacaacaaactggtgcgtcccgtcgtcaacacgtcggacgtcctcagggtctgcatcaagctcaagttgtcccaactcatagacgtgaacctgaagaaccaaatcatgacgacgaatctgtgggtggagcaatcctggtacgactacaaactacgctgggagcccaaggaatacggaggcgtgcacatgctccacgtcccctcggaccacatctggagaccggacattgtgctgtataacaacgccgacggcagctacgaagtgacgataaaaacgaaggctaccgtgtactacacaggcctcgtggtgtggcaaccgccggccgtgtataaatcctcgtgcgcaatagatgtcgaatttttcccatacgacgtacagacgtgcgttttgaagttaggaagttggacctatgacgggtttaaggtggacttacgtcacatggatgagaaagctggctccaacgtggtggaagtgggtgtggacctctccgaattctacatgtctgtcgagtgggacatcttggaggttccggccgtcaggaacgagaaattctacacgtgctgcgacgagccgtacctagacattacgttcaacatcacaatgcggcggaagactcttttctacacagtgaacatcatcatcccttgtatgggcatctcgtttctcaccgtactaacattctacctgccttcggacagcggtgaaaaggtgaccctgtcaatatcaatcctcatcagtctccacgtgttcttcctgctggtcgtcgagatcatcccgcccacctctctcgtggtgccactcctcggcaagtacctcatcttcgccatgattctcgtctccataagcatatgtgttacggttgtggtgctcaacgtacatttccggtcgccacagactcacaagatggcgccttgggtgaagagagttttcatacacatcttaccaagattgctggttatgcggcgccctcagtatcaattcgagaccaacagatacagctctggacgagtgttgatgcgtacagtgagaggtaaggaaattgcaccgtgctactacccataccaacccacggctcagcaacccagcgacgatgacctcaagacatcctgcatgggcagcggacggacatcgaggcaacgttctcctacaaaggatgacctgtctccttccagtttggtagacggtggtcctctcagtggaagctgtcaaattcatggacctgcggtagtgctgcagtctgagtctacagacgaattgtcagacaacaacaagagtccagtgcttcgaaaccctgcattttcacactctgcctgcccgccagaagtgcacaaatcctgcttctgcgttcgcttcatcgcagaacacaccaaaatgctagaggattctaccaaagtaaaggaggactggaagtatgtagcaatggtattagatcgattgtttttgtggattttcacgctagccgtcctggtgggaacagctggtatcatcctacaggcaccaacactctatgatgatcgagttcccattgataagggattcagtgaactagcaacagtagtgcgttgcccaccacagtagcagccttcgttaatgatacatgcatagaattgagcaaagtctgtaaaaccaacaactgacatgccagagttcaattgttagatcgaccaaaatgtatggaattgagagttttatgattatggcctatatcattgcacaatttactggccatagtaagataaataaagtcattgtagtttttattggaaaggtaagaccataataagttggttatttttgcgagttttaagaacatttaatgacgctcaaactaatgtattacggtaccataaggtaatcttatttaagaatggaaatttcagaaatctcgttaatatttcaagacaaagaattaagcaaagcataagtctgattcatgggttcatgctttcatttgaaacatttggatatatttgagagaagaaaagataattacattcttggcctattttattgtaggtactagttgaaaattatctagaaaaaaaaaaaaaaaaaaa

SEQ ID NO: 23 - DNA, 2200 bp, Periplaneta americana
  misc_feature 1..2200: note = alpha4 sub-unit of the nicotinic acetylcholine receptor
  source 1..2200: mol_type = genomic DNA
agtgcgagggatgacgtcgcggtaacgagatggtgtgtgccggggatgcgctgagggcctgggtgctatcggcgctggtggtgcacggggcagtggcgggcaaccccgacgccaagcgcctgtacgacgacctcctcagcaactacaacaaactggtgcgtcccgtcgtcaacacgtcggacgtcctcagggtctgcatcaagctcaagttgtcccaactcatagacgtgaacctgaagaaccaaatcatgacgacgaatgtgtgggtggagcaatcctggcacgactacaaactacgctgggagcccaaggaatatggaggcgtgcacatgctccacgtcccctcggaccacatctggagaccggacattgtgctgtataacaacgctgatggcaacttcgaggttaccctggcaaccaaggcaactatctaccaccaaggactggtggaatggaaaccgccagccatatacaagtcatcttgtgagatagacgtagaatattttccctttgacgagcaaacctgcgttttgaaattcggtagttggacctatgacggcttcaaggtggacttacgtcacatggatgagaaagctggctccaacgtggtggaagtgggtgtggacctctccgaattctacatgtctgtcgagtgggacatcttggaggttccggccgtcaggaacgagaaattctacacatgctgcgacgagccgtatctagacatcacgttcaacatcacaatgcggcggaagactcttttctacacagtgaacatcatcatcccttgtatgggcatctcgtttctcaccgtattaacattctacctgccttcggacagcggtgaaaaggtgaccctgtcaatatcaatcctcatcagtctccacgtgttcttcctgctggtcgtcgagatcatcccgcccacctctctcgtggtgccactcctcggcaagtacctcatcttcgccatgattctcgtctccataagcatatgtgttacggttgtggtgctcaacgtacatttccggtcgccacagactcacaagatggcgccttgggtgaagagagttttcatacacatcttaccaagattgctggttatgcggcgccctcagtatcaattcgagaccaacagatacagctctggacgagtgttgatgcgtacagtgagaggtaaggaaattgcaccgtgctactacccataccaacccacggctcagcaacccagcgacgatgacctcaagacatcctgcatgggcagcggacggacatcgaggcaacgttctcctacaaaggatgacctgtctccttccagtttggtagacggtggtcctctcagtggaagctgtcaaattcatggacctgcggtagtgctgcagtctgagtctacagacgaattgtcagacaacaacaagagtccagtgcttcgaaaccctgcattttcacactctgcctgcccgccagaagtgcacaaatcctgcttctgcgttcgcttcatcgcagaacacaccaaaatgctagaggattctaccaaagtaaaggaggactggaagtatgtagcaatggtattagatcgattgtttttgtggatcttcacgctagccgtcctggtgggaacagctggtatcatcctacaggcaccaacactctatgatgatcgagttcccattgataagggattcagtgaactagcaacagtagtgcgttgcccaccacagtagcagccttcgttaatgatacatgcatagaattgagcaaagtctgtaaaaccaacaactgacatgccagagttcaattgttagatcgaccaaaatgtatggaattgagagttttatgattatggcctatatcattgcacaatttactggccatagtaagataaataaagtcattgtagtttttattggaaaggtaagaccataataagttggttatttttgcgagttttaagaacatttaatgacgctcaaactaatgtattacggtaccataaggtaatcttatttaagaatggaaatttcagaaatctcgttaatatttcaagacaaagaattaagcaaagcataagtctgattcatgggttcatgctttcatttgaaacatttggatatatttgagagaagaaaagataattacattcttggcctattttattgtaggtactagttgaaaattatctagaaaaaaaaaaaaaaaaaaa

SEQ ID NO: 24 - DNA, 1478 bp, Periplaneta americana
  misc_feature 1..1478: note = alpha5 sub-unit of the nicotinic acetylcholine receptor
  source 1..1478: mol_type = genomic DNA
cgccggaaagcatcagtcaatcactgatgccccgcttgatcggcggcagccccgaagtattgctcttcaggtgtgtgtgtccacggccgcggcatatgacggctcttcgcgccatattctgcgtggtcatggtgacaagcgtgctgggagatgaacaggaatatcgactcacgcgctatctcatggccaattacgacccgtcagtgcgcccagcggagaactcttccgaacctctcaaagtggtgttcgggatctcactgcatcatatcatcgatgttgacgagaagaaccagatcctgacaacgaactgctggctcacgcagatatggacggaccaccacctcaagtggaattcatctgacttcgcggggatcaaagtgatcagggtgccataccagcgagtctggaggccggacaccatcctctacaacaatgcggactctcagtacaactcggctgtgattaacacaaacgtcattgtgacgaacaatggggaggtcgtgtggctgagtcacggaatcttccgtagttcctgcgacatcaacgtggagtacttccccttcgacgttcagagctgtaacatgaagtgggcctcttggacgtacgacggttaccagttggacttggtgaaacaaacagaggaaggcgacgtaagcaactaccaggcaaatggcgagttcgacctggtcagtttcgacgcgatccgtcacgtcgagttctactcgtgctgtccggagccttaccccgacatcacgtacgtgattcgactccggaggcgacccatgttctacgtcttcaacctcatcctgccctgcatcctcatcaacggcattgcactacttgtgttctacgttccgtccgagtctggagaaaaggtgactctaggcatcagcgctctgctgtccatgacagtgttccttatgaccatacgagagagtcttccacccactgagaagacacctttaattagtctttattatggagttagcatttgcctagtttcattcgcatcaggcctatctgtggtgacattgaatgtatatcaccgtggtgtgagaggaaccggtgttccaagaattattaaggcccttatacttggcaagcttgcaagacttgtcttccttcagtttgaaccacgagataaacatgggccatccatgcaacatcaggacataaacccaaggacaggtacgcggctagattgtccgcgtccaccggataacaagtggacagcagacaacgaaggggattatctggaccctcaacaaatgtccccccgtttccttcctcgtcgtagtcggacagattcaacaccaggtacagatagttttgaagcgcagtttctgcgtgtgctcaacaaggtgaaccacaccatagagcgcaatgaactccgtcttgcagagcaggaacggcgtgatgtgacggagatagaatggaagcaagtagccttggtgtgtgacaggttcttgctgtgggtg

SEQ ID NO: 25 - DNA, 2358 bp, Periplaneta americana
  misc_feature 1..2358: note = alpha6 sub-unit of the nicotinic acetylcholine receptor
  source 1..2358: mol_type = genomic DNA
atggggaagagaggatgacatcgtcagcccagacactgggactcttcgcatctgctcttctgctgatcattctaccaaaaggcactgagcaaggcacccatgagaagaagctcctcactcaactgctcgagaggtacaacgttctggaacggcctgtagccaacgagtcggagcccctcgaagtgaaattcggcctcacgcttcaacagatcatcgatgtggacgaaaaaaatcaactcctaataacaaatatttggctttcgttggagtggaacgactacaacctcaggtggaacgaatctgagttcggtggagtcaaggacctcaggataactccaaataaaatttggagaccagatatcctcatgtacaacagtgctgatgaaggtttcgacgggacctaccacaccaacgttgtggtcagacgtgacggtggctgcttgtacgttccaccgggcatcttcaagagcacatgcaagatggacattacgtggtttcccttcgatgaccaacactgtgacatgaagttcggtagctggacctatgatggcaaccagttggacctagttctcaactcagaagagggtggagatctctccgacttcatcaccaacggagaatggtatctaataggaatgcctggtaagaagaacaccatcgtttaccagtgctgtccggagccttacgtcgatgtgaccttcaccattcagatccgaaggcgaactctttactacttcttcaacctgattgtgccctgcgtgctgatctcgtccatggcgctgctgggcttcacccttccacccgactccggggagaagctcaccctaggagtgaccatcttgctctcgttgacagtcttcctgaacctggtcgccgagaccctcccccaagtctctgacgccattcctctgttagggacatactttaactgtataatgtttatggtggcctcgtccgtcgtgctcactgtggtggttctcaactaccaccatcggaccgcagacattcatgagatgccgcaatggatcaagagcgtattcttgcagtggctgccctggattctcggaatgagtcgtccggggaggaagatcacgaggaaaaccatcctcatgagcaatagaatgaaggagctggagctgaaagagcgttcctccaagtccctgctagcgaatgtgctggacattgacgacgactttcgtcattgctccggaaccaacagcacgtcggcgacgtccgccgggttcctgcgaagcggagggcgaggcaccgcggcaacgctcgacgacaccgggagcacgccttgctccgtggctcagagggagctccaggccatcctgagggaactgcagttcatcacgggcaggatgaggaaggcggacgatgaggcggagctcataagcgattggaagttcgccgcgatggtggtcgataggttctgcctcataatcttcaccatgttcaccataatcgctactgtagcagtacttctgtcggcgccccatatcatcgttcagtagcccctcgtcatcatgttcgccgccattccatccagttttctccccaggcaccgtgcgtttccaatatgtcgcagatattttttgtgcgtaaatgtagagtcgatattagggttaaaggcgaagaggaaccaaagcgcacaaagccgaaaaatgccgcggtctcaacccggcagatttcttctctgtctactacgattgactacggtagaagcgtcaatatactatcaaaaattgctttataattgaaaccatatgcttaatattataacggatcaattgcggggaacagttctaagaagtctcttctaatggagtgagcccgtacagattttaacacaatcactacgataaaatgagaatcgaaagattttatcattatagtgattcatatcaaacgatattttataatacttcatactaatgaacgagtaaatctgatagaaaagcaatgaactaacttctttacacttggggccaaaaataattgtgagggtaaaatcgtatgttcggccaggtaactgtgaagtcgggataacattaactagataacgttcaagtgtagagttagactgttgatattataccactcgcgtgtgtctcccgaaacgcgatcaatacacaaacatttctgtgatatctacgatttgtttttttctttcttttaacccttgaataccaaaacacaggcaaactaaatcaaaggaagataattaacacgtattttcattcagttagccagaattactggattgtgaaaccgtgtacaaaaggaattgaagaaataactccatttctgtacttttgtataaatgaaaatactacaaaactaaaaaaaaaaaaa

SEQ ID NO: 26 - DNA, 2967 bp, Periplaneta americana
  misc_feature 1..2967: note = alpha6 sub-unit of the nicotinic acetylcholine receptor
  source 1..2967: mol_type = genomic DNA
cccacaacaagcaccgccacatgcaaacacattcttgtcaagtttctcagggaagaaagttgcatcggtacgagagcaagaagaaaaaccatcagcagatccgagagccttgggcaccagcgcatcgcgagtagttgacgttattttctatccgcacccggctagcgcaccggctgtaccagtgtctgctctttgtttgcttgagtcgcctcaccgacaggtcgtccgcaacagacggcgcggcatcggggcttccaaaacattcctcacagttcggcacgatatcctggttccagtagcagtaactccatgaagtgacagctgtagaacactgagacacgaatctgttagctcttctccgaacgtttattcgttttactacagtgtaagaagtggaaaattattcctgtataaaacattgttatgacggccaaatctggtggactcttggcagggaaacgacttccgttatgacaggtttcgagtctataaaaggagctttcatatgcgaaaactgagagcgtcgcggtggagggaagtaaaaccggaaagagtttgtgagaaaagtagctgtagcagggaggagattgccttctccagaagcaggaagaggggatgacatcgtcagcccagacactgggactcttcgcatctgctcttctgctgatcattctaccaaaaggcactgagcaaggcacccatgagaagaagctcctcactcaactgctcgagaggtacaacgttctggaacggcctgtagccaacgagtcggagcccctcgaagtgaaattcggcctcacacttcaacagatcatagacgtggacgaaaagaatcaaattctgacaacaaacgcttggttgaatttggagtggaacgactacaacctcaggtggaacgaatctgagttcggtggagtcaaggacctcaggataactccaaataaaatttggagaccagatatcctcatgtacaacagtgctgatgaaggtttcgacgggacctaccacaccaacgttgtggtcaggcatggcggtggctgcttgtacgttccaccgggcatcttcaagagcacatgcaagatggacattacgtggttcccctttgatgaccaacattgtgacatgaagttcggtagctggacctatgatggcaaccagttggacctagttctcaactcagaagagggtggagatctctccgacttcatcaccaacggagaatggtatctaataggaatgcctggtaagaagaacaccatcgtttaccagtgctgtccggagccttacgtcgatgtgaccttcaccattcagatccgaaggcgaactctttactacttcttcaacctgattgtgccctgcgtgctgatctcgtccatggcgctgctgggcttcacccttccacccgactccggggagaagctcaccctaggagtgaccatcttgctctcgttgacagtcttcctgaacctggtcgccgagaccctcccccaagtctctgacgccattcctctgttagggacatacttcaactgtatcatgttcatggtggcctcgtccgtcgtgctcactgtggtggttctcaactaccaccatcggaccgcagacattcatgagatgccgcaatggatcaagagcgtattcttgcagtggctgccctggattctcggaatgagtcgtccggggaagaagatcacgaggaaaaccatcctcatgagcaatagaatgaaggagctggagctgaaagagcgttcctccaagtccctgctagcgaatgtgctggacattgacgacgactttcgtcattgctccggaaccaacagcacgtcggcgacgtccgccgggttcctgcgaagcggagggcgaggcaccgcggcaacgctcgacgacaccgggagcacgccttgctccgtggctcagagggagctccaggccatcctgagggaactgcagttcatcacgggcaggatgaggaaggcggacgatgaggcggagctcataagcgactggaagttcgccgcgatggtggtcgataggttctgcctcataatcttcaccatgttcaccataatcgctactgtagcagtacttctgtcggcgccccatatcatcgttcagtagcccctcgtcatcatgttcgccgccattccatccagttttctccccaggcaccgtgcgtttccaatatgtcgcagatattttttgtgcgtaaatgtagagtcgatattagggttaaaggcgaagaggaaccaaagcgcacaaagccgaaaaatgccgcggattcaacccggcagatttcttctctgtctactacgattgactacggtagaagcgtcaatatactatcaaaaattgctttataattgaaaccatatgcttaatattataacggatcaattgcggggaacagttctaagaagtctcttctaatggagtgagcccatacagattttaacacaatcactacgataaaatgagaatcgaaagattttatcattatagtgattcatatcaaacgatattttataatacttcatactaatgaacgagtaaatctgatagaaaagcaatgaactaacttctttacacttggggccaaaaataattgtgagggtaaaatcgtatgttcggccaggtaactgtgaagtcgggataacattaactagatagcgttcaagtgtagagttagactgttgatactataccactcgcgtgtgtctcccgaaacgcgatcaatacacaaacattcctgtgatacctacgatttgtttttttctttcttttaacccttgaataccaaaacacaggcaaactaaatcaaaggaagataattaacacgtattttcattcagttagccataattactggattgtgaaaccgtgtacaaaaggaattgaagaaataactccatttctgtacttttgtataaatgaaaatactacaaaactaaaaaaaaaaaaaaaaaaaaa

SEQ ID NO: 27 - DNA, 2967 bp, Periplaneta americana
  misc_feature 1..2967: note = alpha6 sub-unit of the nicotinic acetylcholine receptor
  source 1..2967: mol_type = genomic DNA
cccacaacaagcaccgccacatgcaaacacattcttgtcaagtttctcagggaagaaagttgcatcggtacgagagcaagaagaaaaaccatcagcagatccgagagccttgggcaccagcgcatcgcgagtagttgacgttattttctatccgcacccggctagcgcaccggctgtaccagtgtctgctctttgtttgcttgagtcgcctcaccgacaggtcgtccgcaacagacggcgcggcatcggggcttccaaaacattcctcacagttcggcacgatatcctggttccagtagcagtaactccatgaagtgacagctgtagaacactgagacacgaatctgttagctcttctccgaacgtttattcgttttactacagtgtaagaagtggaaaattattcctgtataaaacattgttatgacggccaaatctggtggactcttggcagggaaacgacttccgttatgacaggtttcgagtctataaaaggagctttcatatgcgaaaactgagagcgtcgcggtggagggaagtaaaaccggaaagagtttgtgagaaaagtagctgtagcagggaggagattgccttctccagaagcaggaagaggggatgacatcgtcagcccagacactgggactcttcgcatctgctcttctgctgatcattctaccaaaaggcactgagcaaggcacccatgagaagaagctcctcactcaactgctcgagaggtacaacgttctggaacggcctgtagccaacgagtcggagcccctcgaagtgaaattcggcctcacacttcaacagatcatagacgtggacgaaaagaatcaaattctgacaacaaacgcttggttgaatttggagtggaacgactacaacctcaggtggaacgaatctgagttcggtggagtcaaggacctcaggataactccaaataaaatttggagaccagatatcctcatgtacaacagtgctgatgaaggtttcgacgggacctaccacaccaacgttgtggtcagacataacggtggctgcttgtacgttccaccgggcatcttcaagagcacgtgcaagatagacattacgtggttcccctttgatgaccaacattgtgacatgaagttcggtagctggacctatgatggcaaccagttggacctagttctcaactcagaagagggtggagatctctccgacttcatcaccaacggagaatggtatctaataggaatgcctggtaagaagaacaccatcgtttaccagtgctgtccggagccttacgtcgatgtgaccttcaccattcagatccgaaggcgaactctttactacttcttcaacctgattgtgccctgcgtgctgatctcgtccatggcgctgctgggcttcacccttccacccgactccggggagaagctcaccctaggcgtcaccatactcctttcgttaaccgtatttctcaacttagtagcagaaaagatgccctccacatctgatgccgtaccattgatagggacatacttcaactgtatcatgttcatggtggcctcgtccgtcgtgctcactgtggtggttctcaactaccaccatcggaccgcagacattcatgagatgccgcaatggatcaagagcgtattcttgcagtggctgccctggattctcggaatgagtcgtccggggaagaagatcacgaggaaaaccatcctcatgagcaatagaatgaaggagctggagctgaaagagcgttcctccaagtccctgctagcgaatgtgctggacattgacgacgactttcgtcattgctccggaaccaacagcacgtcggcgacgtccgccgggttcctgcgaagcggaggacgaggcaccgcggcaacgctcgacgacaccgggagcacgccttgctccgtggctcagagggagctccaggccatcctgagggaactgcagttcatcacgggcaggatgaggaaggcggacgatgaggcggagctcataagcgactggaagttcgccgcgatggtggtcgataggttctgcctcataatcttcaccatgttcaccataatcgctactgtagcagtacttctgtcggcgccccatatcatcgttcagtagcccctcgtcatcatgttcgccgccattccatccagttttctccccaggcaccgtgcgtttccaatatgtcgcagatattttttgtgcgtaaatgtagagtcgatattagggttaaaggcgaagaggaaccaaagcgcacaaagccgaaaaatgccgcggattcaacccggcagatttcttctctgtctactacgattgactacggtagaagcgtcaatatactatcaaaaattgctttataattgaaaccatatgcttaatattataacggatcaattgcggggaacagttctaagaagtctcttctaatggagtgagcccatacagattttaacacaatcactacgataaaatgagaatcgaaagattttatcattatagtgattcatatcaaacgatattttataatacttcatactaatgaacgagtaaatctgatagaaaagcaatgaactaacttctttacacttggggccaaaaataattgtgagggtaaaatcgtatgttcggccaggtaactgtgaagtcgggataacattaactagatagcgttcaagtgtagagttagactgttgatactataccactcgcgtgtgtctcccgaaacgcgatcaatacacaaacattcctgtgatacctacgatttgtttttttctttcttttaacccttgaataccaaaacacaggcaaactaaatcaaaggaagataattaacacgtattttcattcagttagccataattactggattgtgaaaccgtgtacaaaaggaattgaagaaataactccatttctgtacttttgtataaatgaaaatactacaaaactaaaaaaaaaaaaaaaaaaaaa

SEQ ID NO: 28 - DNA, 2967 bp, Periplaneta americana
  misc_feature 1..2967: note = alpha6 sub-unit of the nicotinic acetylcholine receptor
  source 1..2967: mol_type = genomic DNA
cccacaacaagcaccgccacatgcaaacacattcttgtcaagtttctcagggaagaaagttgcatcggtacgagagcaagaagaaaaaccatcagcagatccgagagccttgggcaccagcgcatcgcgagtagttgacgttattttctatccgcacccggctagcgcaccggctgtaccagtgtctgctctttgtttgcttgagtcgcctcaccgacaggtcgtccgcaacagacggcgcggcatcggggcttccaaaacattcctcacagttcggcacgatatcctggttccagtagcagtaactccatgaagtgacagctgtagaacactgagacacgaatctgttagctcttctccgaacgtttattcgttttactacagtgtaagaagtggaaaattattcctgtataaaacattgttatgacggccaaatctggtggactcttggcagggaaacgacttccgttatgacaggtttcgagtctataaaaggagctttcatatgcgaaaactgagagcgtcgcggtggagggaagtaaaaccggaaagagtttgtgagaaaagtagctgtagcagggaggagattgccttctccagaagcaggaagaggggatgacatcgtcagcccagacactgggactcttcgcatctgctcttctgctgatcattctaccaaaaggcactgagcaaggcacccatgagaagaagctcctcactcaactgctcgagaggtacaacgttctggaacggcctgtagccaacgagtcggagcccctcgaagtgaaattcggcctcacacttcaacagatcatagacgtggacgaaaagaatcaaattctgacaacaaacgcttggttgaatttggagtggaacgactacaacctcaggtggaacgaatctgagttcggtggagtcaaggacctcaggataactccaaataaaatttggagaccagatatcctcatgtacaacagtgctgatgaaggtttcgacgggacctaccacaccaacgttgtggtcagacatgacggtggctgcttgtacgttccaccgggcatcttcaagagcacatgcaagatagacattacgtggttcccctttgatgaccaacattgtgtcatgaagttcggtagctggacctatgatggcaaccagttggacctagttctcaactcagaagagggtggagatctctccgacttcatcaccaacggagaatggtatctaataggaatgcctggtaagaagaacaccatcgtttaccagtgctgtccggagccttacgtcgatgtgaccttcaccattcagatccgaaggcgaactctttactacttcttcaacctgattgtgccctgcgtgctgatctcgtccatggcgctgctgggcttcacccttccacccgactccggggagaagctcaccctaggtgtaacaatcctgctgtctctaacagttttctccctcctggtggcccaagtgttgccacagacttctgacgctgtgcccctgatagggacatacttcaactgtatcatgttcatggtggcctcgtccgtcgtgctcactgtggtggttctcaactaccaccatcggaccgcagacattcatgagatgccgcaatggatcaagagcgtattcttgcggtggctgccctggattctcggaatgagtcgtccggggaagaagatcacgaggaaaaccatcctcatgagcaatagaatgaaggagctggagctgaaagagcgttcctccaagtccctgctagcgaatgtgctggacattgacgacgactttcgtcattgctccggaaccaacagcacgtcggcgacgtccgccgggttcctgcgaagcggagggcgaggcaccgcggcaacgctcgacgacaccgggagcacgccttgctccgtggctcagagggagctccaggccatcctgagggaactgcagttcatcacgggcaggatgaggaaggcggacgatgaggcggagctcataagcgactggaagttcgccgcgatggtggtcgataggttcggcctcataatcttcaccatgttcaccataatcgctactgtagcagtacttctgtcggcgccccatatcatcgttcagtagcccctcgtcatcatgttcgccgccattccatccagttttctccccaggcaccgtgcgtttccaatatgtcgcagatattttttgtgcgtaaatgtagagtcgatattagggttaaaggcgaagaggaaccaaagcgcacaaagccgaaaaatgccgcggattcaacccggcagatttcttctctgtctactacgattgactacggtagaagcgtcaatatactatcaaaaattgctttataattgaaaccatatgcttaatattataacggatcaattgcggggaacagttctaagaagtctcttctaatggagtgagcccatacagattttaacacaatcactacgataaaatgagaatcgaaagattttatcattatagtgattcatatcaaacgatattttataatacttcatactaatgaacgagtaaatctgatagaaaagcaatgaactaacttctttacacttggggccaaaaataattgtgagggtaaaatcgtatgttcggccaggtaactgtgaagtcgggataacattaactagatagcgttcaagtgtagagttagactgttgatactataccactcgcgtgtgtctcccgaaacgcgatcaatacacaaacattcctgtgatacctacgatttgtttttttctttcttttaacccttgaataccaaaacacaggcaaactaaatcaaaggaagataattaacacgtattttcattcagttagccataattactggattgtgaaaccgtgtacaaaaggaattgaagaaataactccatttctgtacttttgtataaatgaaaatactacaaaactaaaaaaaaaaaaaaaaaaaaa

SEQ ID NO: 29 - DNA, 2880 bp, Periplaneta americana
  misc_feature 1..2880: note = alpha6 sub-unit of the nicotinic acetylcholine receptor
  source 1..2880: mol_type = genomic DNA
cccacaacaagcaccgccacatgcaaacacattcttgtcaagtttctcagggaagaaagttgcatcggtacgagagcaagaagaaaaaccatcagcagatccgagagccttgggcaccagcgcatcgcgagtagttgacgttattttctatccgcacccggctagcgcaccggctgtaccagtgtctgctctttgtttgcttgagtcgcctcaccgacaggtcgtccgcaacagacggcgcggcatcggggcttccaaaacattcctcacagttcggcacgatatcctggttccagtagcagtaactccatgaagtgacagctgtagaacactgagacacgaatctgttagctcttctccgaacgtttattcgttttactacagtgtaagaagtggaaaattattcctgtataaaacattgttatgacggccaaatctggtggactcttggcagggaaacgacttccgttatgacaggtttcgagtctataaaaggagctttcatatgcgaaaactgagagcgtcgcggtggagggaagtaaaaccggaaagagtttgtgagaaaagtagctgtagcagggaggagattgccttctccagaagcaggaagaggggatgacatcgtcagcccagacactgggactcttcgcatctgctcttctgctgatcattctgccaaaaggcactgagcaaggcacccatgagaagaagctcctcactcaactgctcgagaggtacaacgttctggaacggcctgtagccaacgagtcggagcccctcgaagtgaaattcggcctcacgcttcaacagatcatagacgtggacgaaaagaatcaaattctgacaacaaacgcttggttgaatttggagtggaacgactacaacctcaggtggaacgaatctgagttcggtggagtcaaggacctcaggataactccaaataaaatttggagaccagatatcctcatgtacaacagtgctgatgaaggtttcgacgggacctaccacaccaacgttgtggtcagacgtgacggtggctgcttgtacgttccaccgggcatcttcaagagcacatgcaagatggacattacgtggttcccctttgatgaccaacattgtgacatgaagttcggtagctggacctatgatggcaaccagttggacctagttctcaactcagaagagggtggagatctctccgacttcatcaccaacggagaatggtatctaataggaatgcctggtaagaagaacaccatcgtttaccagtgctgtccggagccttacgtcgatgtgaccttcaccattcagatccgaaggcgaactctttactacttcttcaacctgattgtgccctgcgtgctgatctcgtccatggcgctgctgggcttcacccttccacccgactccggggagaagctcaccctagggacatacttcaactgcatcatgttcatggtggcctcgtccgtcgtgctcactgtggtggttctcaactaccaccatcggaccgcagacattcatgagatgccgcaatggatcaagagcgtattcttgcagtggctgccctggattctcggaatgagtcgtccggggaagaagatcacgaggaaaaccatcctcatgagcaatagaatgaaggagctggagctgaaagagcgttcctccaagtccctgctagcgaatgtgctggacattgacgacgaccttcgtcattgctccggaaccaacagcacgtcggcgacgtccgccgggttcctgcgaagcggagggcgaggcaccgcggcaacgctcgacgacaccgggagcacgccttgctccgtggctcagagggagctccaggccatcctgagggaactgcagttcatcacgggcaggatgaggaaggcggacgatgaggcggagctcataagcgactggaagttcgccgcgatggtggtcgataggttctgcctcataatcttcaccatgttcaccataatcgctactgtagcagtacttctgtcggcgccccatatcatcgttcagtagcccctcgtcatcatgttcgccgccattccatccagttttctccccaggcaccgtgcgtttccaatatgtcgcagatattttttgtgcgtaaatgtagagtcgatattagggttaaaggcgaagaggaaccaaagcgcacaaagccgaaaaatgccgcggattcaacccggcagatttcttctctgtctactacgattgactacggtagaagcgtcaatatactatcaaaaattgctttataattgaaaccatatgcttaatattataacggatcaattgcggggaacagttctaagaagtctcttctaatggagtgagcccatacagattttaacacaatcactacgataaaatgagaatcgaaagattttatcattatagtgattcatatcaaacgatattttataatacttcatactaatgaacgagtaaatctgatagaaaagcaatgaactaacttctttacacttggggccaaaaataattgtgagggtaaaatcgtatgttcggccaggtaactgtgaagtcgggataacattaactagatagcgttcaagtgtagagttagactgttgatactataccactcgcgtgtgtctcccgaaacgcgatcaatacacaaacattcctgtgatacctacgatttgtttttttctttcttttaacccttgaataccaaaacacaggcaaactaaatcaaaggaagataattaacacgtattttcattcagttagccataattactggattgtgaaaccgtgtacaaaaggaattgaagaaataactccatttctgtacttttgtataaatgaaaatactacaaaactaaaaaaaaaaaaaaaaaaaaa

SEQ ID NO: 30 - DNA, 1497 bp, Periplaneta americana
  misc_feature 1..1497: note = alpha7 sub-unit of the nicotinic acetylcholine receptor
  source 1..1497: mol_type = genomic DNA
atgccccgatggcttctcttcgtctgcatatgcatttgtcttccacgcgagtcgctacaagggccccacgaaaagcgattgcttcacaccctactggatcattacaacgtcctggagcgacccgtagccaatgagtcggaccctcttcagctcagtttcggtctcacgctaatgcaaattatagatgtggatgagaagaatcagcttctaataacaaacatatggttaaaactggaatggaatgatgtcaatcttcggtggaactcgacagagtatggcggcgttaaagacctcaggatacctccacatcggatatggaaaccagacgtgctcatgtacaacagtgccgatgagggtttcgacgggacgtatccgacaaacgttgtggtcagggatagcggtagctgcttgtacgttccaccgggcatcttcaagagcacatgcaagatagacattacgtggttcccctttgatgaccaacgctgtgagatgaagttcggtagctggacctatgatggctttcagttggatctacaattgcaagacgacaccggtggggacatcagcagcttcataacgaatggtgaatgggacctcttaggtgttcccgggaaacggaacgaaatttactacaattgctgccccgaaccttatatagacataacttttatcattatcatacgacgccggaccctgtactactttttcaaccttatagttccatgtgttctcatcgcctccatggctgttctgggctttactctgccaccggactcaggcgaaaagctatctttaggtgttacaatcctcctctctctgactgtattcctaaacatggtcgctgagacgatgcctgccacatccgatgctgtccctttattaggtacatatttcaactgtattatgttcatggtggcttcttccgtcgtttcaactatcctgatactaaactatcatcatagaaatgcagatacacatgaaatgtctccttggataaaaacgatcttcttgcactggctgccttgcatcctgcggatgagcaggccgggcggcgagcccgacgcctccaggaagaacctgcagatgcgcgagctggagctcaaggagcgctcgtccaagagcctgctcgccaacgtgctcgacatcgacgacgacttccggcaccacaccggcaccctgcagacgtcggcggcgggcttcctccgaggcgcccacacgcatcacgaggacggcgggggcggcggggggacgctcctgcacgggggcggcgggggctcctcgtgcctgggcccccaccgcgaactaaccctcatcctcaaggagctgcgcgtgatcacggacaagttgagaaaagaggacgaggcggcggaagtgacgaacgactggaagttcgccgccatggtggtggatagactgtgcctcataatcttcactctgttcacagttatcgcgactatcgcggtcctcttctccgcgccccacatcatcgtgacgtaa

SEQ ID NO: 31 - DNA, 2831 bp, Periplaneta americana
  misc_feature 1..2831: note = alpha7 sub-unit of the nicotinic acetylcholine receptor
  source 1..2831: mol_type = genomic DNA
atgggggtgcgagctctgagagggacgagtcggctgaacaccgtcatcgcggctgtggtgcaggaagacaaggagaagagaggaagagagcggagtagcgaccagcagatccggaagccggcagttggagggataccaccagcacaccaatgtattccgagtacagcaggtgaagaagaacaccatggagtcaacagcagcctccgaggcctgctcaacagaccaagggcctcctctgccagtcatgccccgatggcttctcttcgtctgcatatgcatttgtcttccacgcgagtcgctacaagggccccacgaaaagcgattgcttcacaccctactggatcattacaacgtcctggagcgacccgtagccaatgagtcggaccctcttcagctcagtttcggtctcgcgctaatgcaaattatagatgtggatgagaagaatcagcttctaataacaaacatatggttaaaactggaatggaatgatgtcaatcttcggtggaactcgacagagtatggcggcgttaaagacctcaggatacctccacatcggatatggaaaccagacgtgctcatgtacaacagtgcagatgagggtttcgacgggacgtatccgacaaacgttgtggtcagggatagcggtagctgcttgtacgttccaccgggcatcttcaagagcacatgcaagatagacattacgtggttcccctttgatgaccaacgctgtgagatgaagttcggtagctggacctatgatggctttcagttggatctacaattgcaagacgacaccggtggggacatcagcagcttcataacgaatggtgaatgggacctcttaggtgttcccgggaaacggaacgaaatttactacaattgctgccccgaaccttatatagacataacttttatcattatcatacgacgccggaccctgtactactttttcaaccttatagttccatgtgttctcatcgcctccatggctgttctgggctttactctgccaccggactcaggcgaaaagctatctttaggtgttacaatcctcctctctctgactgtattcctaaacatggtcgctgagacaatgcctgccacatccgatgctgtccctttattaggtacatatttcaactgtattatgttcatggtggcttcttccgtcgtttcaactatcctgatactaaactatcatcatagaaatgcagatacacatgaaatgtctccttggataaaaacgatcttcttgcactggctgccttgcatcctgcggatgagcaggccgggcggcgagcccgacgcctccaggaagaacctgcagatgcgcgagctggagctcaaggagcgctcgtccaagagcctgctcgccaacgtgctcgacatcgacgacgacttccggcaccacaccggcaccctgcagacgtcggcggcgggcttcctccgaggcgcccacacgcatcacgaggacggcgggggcggcggggggacgctcctgcacgggggcggcgggggctcctcgtgcctgggcccccaccgcgaactaaccctcatcctcaaggagctgcgcgtgatcacggacaagttgagaaaagaggacgaggcggcggaagtgacgaacgactggaagttcgccgccatggtggtggatagactgtgcctcataatcttcactctgttcacagttatcgcgactatcgcggtcctcttctccgcgccccacatcatcgtgacgtaatcgtcgcgcgcgcgcggcatttgttatcttttattcattattgtatagtattggcaatagtgcatccccgacaatggagcatcacaccaaatcacgtagcaacacgtgatgctgcggactccaacggatggacggacagacggacagacagactgatggaagtggacgcgactttctaaccgctagggatcttacaagaggttttagtaatataatagttttgagtctagaggagggcgttatttatattacacatgactatcacacaagttaagtatgtggactgactaccacaatacaatgactggttgtattgtgagaggaggtgaaatcattttacgaatcgcaatacaacgattggttgtattgtcggccaccaagaggcaaacaatccaatgacttgttcatagttattaataaaaaaaattaattaatattgcgtcaccacagcaaacaaaagtgtgtggtttccatgaatcgcaatacaataagatgaagttgtattgtagggatatctcgagaacacgcaatacaataaataggtattgttgagctttgagaattttggttgtgagtaaatggttgattgttgttttacaatttatgaacgggttattcaaatgattcttctgcgcatttatactgctaaacacaatgtacggacaatgttaactgaatcttttttgtgaggctattcgatctagtcatcgttttgatgacttttttaataggtacgttttttattgaaacaatgaactaggaatttggtatttattatgaataccacaacaagttagtaggacgattcatagctgatgtttatattgtaactaaacatcacagtcgtagttacgttgtagcgacctgcattataatgttgttcacaatttttttaagaaaaacaaaagagagtgaaactgaaatttaactacacataccatcaagctgcgaatgtgaaggtgaaaaggaaaacagtgatacagacatcaatacacacgcatacactaaagcggagaagcttgcagatatgtgtaactttatatgaagagtcgatcattcgtggaatcacatcttccgtaaaaaaaaaaaa

SEQ ID NO: 32 - DNA, 1557 bp, Periplaneta americana
  misc_feature 1..1557: note = alpha7 sub-unit of the nicotinic acetylcholine receptor
  source 1..1557: mol_type = genomic DNA
atggagtcaacagcagcctccgaggcctgctcaacagaccaagggcctcctctgccagtcatgccccgatggcttctcttcgtctgcatatgcatttgtcttccacgcgagtcgctacaagggccccacgaaaagcgattgcttcacaccctactggatcattacaacgtcctggagcgacccgtagccaatgagtcggaccctcttcagctcagtttcggtctcacgctaatgcaaattatagatgtggatgagaagaatcagcttctaataacaaacatatggttaaaactggaatggaatgatgtcaatcttcggtggaactcgacagagtatggcggcgttaaagacctcaggatacctccacatcggatatggaaaccagacgtgctcatgtacaacagtgccgatgagggtttcgacgggacgtatccgacaaacgttgtggtcagggatagcggtagctgcttgtacgttccaccgggcatcttcaagagcacatgcaagatagacattacgtggttcccctttgatgaccaacgctgtgagatgaagttcggtagctggacctatgatggctttcagttggatctacaattgcaagacgacaccggtggggacatcagcagcttcataacgaatggtgaatgggacctcttaggtgttcccgggaaacggaacgaaatttactacaattgctgccccgaaccttatatagacataacttttatcattatcatacgacgccggaccctgtactactttttcaaccttatagttccatgtgttctcatcgcctccatggctgttctgggctttactctgccaccggactcaggcgaaaagctatctttaggtgttacaatcctcctctctctgactgtattcctaaacatggtcgctgaggcgatgcctgccacatccgatgctgtccctttattaggtacatatttcaactgtattatgttcatggtggcttcttccgtcgtttcaactatcctgatactaaactatcatcatagaaatgcagatacacatgaaatgtctcctaggataaaaacgatcttcttgcactggctgccttgcatcctgcggatgagcaggccgggcggcgagcccgacgcctccaggaagaacctgcagatgcgcgagctggagctcaaggagcgctcgtccaagagcctgctcgccaacgtgctcgacatcgacgacgacttccggcaccacaccggcaccctgcagacgtcggcggcgggcttcctccgaggcgcccacacgcatcacgaggacggcgggggcggcggggggacgctcctgcacgggggcggcgggggctcctcgtgcctgggcccccaccgcgaactaaccctcatcctcaaggagctgcgcgtgatcacggacaagttgagaaaagaggacgaggcggcggaagtgacgaacggctggaagttcgccgccatggtggtggatagactgtgcctcataatcttcactctgttcacagttatcgcgactatcgcggtcctcttctccgcgccccacatcatcgtgacgtaa

SEQ ID NO: 33 - DNA, 1920 bp, Periplaneta americana
  misc_feature 1..1920: note = alpha7 sub-unit of the nicotinic acetylcholine receptor
  source 1..1920: mol_type = genomic DNA
cttgcctactgggctttggtgcgggaaagggctttactggtggactggtccaggaagatgctgcgctctgcgatgtcgtgcgagctctgagagggacgagtcggctgaacaccgtcatcgcggctgtggtgtaggaagacaaggagaagagaggaagagagcggagtagcgaccagcagatccggaagccggcagttggagggataccaccagcacaccaatgtattccgagtacagcaggtgaagaagaacaccatggagtcaacagcagcctccgaggcctgctcaacagaccaagggcctcctctgccagtcatgccccgatggcttctcttcgtctgcatatgcatttgtcttccacgcgagtcgctacaagggccccacgaaaagcgattgcttcacaccctactggatcattacaacgtcctggagcgacccgtagccaatgagtcggaccctcttcagctcagtttcggtctcacgctaatgcaaattatagatgtggatgagaagaatcagcttctaataacaaacatatggttaaaactggaatggaatgatgtcaatcttcggtggaactcgacagagtatggcggcgttaaagacctcaggatacctccacatcggatatggaaaccagacgtgctcatgtacaacagtgccgatgagggtttcgacgggacgtatccgacaaacgttgtggtcagggatagcggtagctgcttgtacgttccaccgggcatcttcaagagcacatgcaagatagacattacgtggttcccctttgatgaccaacgctgtgagatgaagttcggtagctggacctatgatggctttcagttggatctacaattgcaagacgacaccggtggggacatcagcagcttcataacgaatggtgaatgggacctcttaggtgttcccgggaaacggaacgaaatttactacaattgctgccccgaaccttatatagacataacttttatcattatcatacgacgccggaccctgtactactttttcaaccttatagttccatgtgttctcatcgcctccatggctgttctgggctttactctgccaccggactcaggcgaaaagctatctttaggtgttacaatcctcctctctctgactgtattcctaaacatggtcgctgagacgatgcctgccacatccgatgctgtccctttattaggtacatacttcaattgcatcatgttcatgttcatggtggcttcttccgtcgtttcaactatcctgatactaaactatcatcatagaaatgcagatacacatgaaatgtctccttggataaaaacgatcttcttgcactggctgccttgcatcctgcggatgagcaggccgggcggcgagcccgacgcctccaggaagaacctgcagatgcgcgagctggagctcaaggagcgctcgtccaagagcctgctcgccaacgtgctcgacatcgacgacgacttccggcaccacaccggcaccctgcagacgtcggcggcgggcttcctccgaggcgcccacacgcatcacgaggacggcgggggcggcggggggacgctcctgcacgggggcggcgggggctcctcgtgcctgggcccccaccgcgaactaaccctcatcctcaaggagctgcgcgtgatcacggacaagttgagaaaagaggacgaggcggcggaagtgacgagcgactggaagttcgtcgccatggtggtggatagactgtgcctcataatcttcactctgttcacagttatcgcgactatcgcggtcctcttctccgcgccccacatcatcgtgacgtaatcgtcgcgcgcgcgcggcatttgttatcttttattcattattgtatagtattggcaatagtgcatccccgacaatggagcatcacaccaaggcgaatcgtac

SEQ ID NO: 34 - DNA, 1623 bp, Periplaneta americana
  misc_feature 1..1623: note = alpha8 sub-unit of the nicotinic acetylcholine receptor
  source 1..1623: mol_type = genomic DNA
atggtaatacttcgaatgcgtcggattctactcttcttcagtttcctaaccattacaacagggaagaccacgacctatgaggccaatccagacgccaagcggctgtacgacgacctgctgagcaactacaaccggctgatccgccccgtcatcaacaacacggagaccctcacagtctggctgggcctcaagctgtcccagctcatagaagtgaacttgaaaaatcaagtaatgacaacaaacctatgggtccaacagaaatggatcgattacaaacttcgctgggacccagaggaatacgggggcgtggagatgctgtacgtcccctccgaacacatctggctgcccgacatcgtgctgtataacaacgctgatggcaactacgaagtgacgctgatgacgaaggcgactctgaagcacaccggcgaagtgttctggaagcctcctgcaatctacaagtcttcctgcgagatcaacgtcgagtacttcccatttgatgaacagagctgcataatgaaattcggatcctggacttacaatggcttccaggttgacttgaaacacatggaccaggtgccaggcagcaacttggtgaacgtgggcattgacctcagcgagttctacctgtccgtggagtgggacatcctggaagtgccggcccgcaggaatgaggagtactatccctgctgcgccgagccctattcagacatcaccttcaacctgacgatgaggcgcaagactctgttctacaccgtgaacctcatcatcccctgcgtgggcatcaccttcctcacggtgctggtgttctacctgccatcggactcgggcgagaaggtgtcgctgtgcgtgtccatcctgctgtcgctcaccgtgttcttcctgctgctggcggagatcatcccgcccacctcgctggccgtgcccctgctgggcaagtacctgctcttcaccatgatcctggtcacgctctccatctccatcaccgtctttgtcctcaacgtccatttccgttccccgtctactcacaagatgtctccgtgggtgaagcgcgtattcttgcactttatgccccgcctgctgatgatgaggcgtccgccatattcttctcgagacaacttcgatgatacaaacttccaagacaacggctacacaaatgaaatggacttcagagatagcataagcgacccatatccgccggagttgaaaggaagtccaggatttgaatctgtgtcaacggcctacaagactgcattgagccaggatgaggaagcgaggcagcatcagccgcattcttccgtcacagactccgacaatactctgccccgtcagctgttgcctgacgtgttgtccgcacttcagggcgtgcgcttcatcgcccagcacatcaaagacgcagacaaagataatgagatcgtggaagactggaagtacgtgtccatggtgctcgaccgcttcttcctgtgggtctttactcttgcctgcataggtggaacctgcggaatcattttccaggcgccgtcactctacgacaaacgcaagcccatcgaccaagagctatctgatataccgctgcgcaagaacaacttccaggttcctccggatattctcaaccagagcgaataa

SEQ ID NO: 35 - DNA, 1392 bp, Periplaneta americana
  misc_feature 1..1392: note = alpha9 sub-unit of the nicotinic acetylcholine receptor
  source 1..1392: mol_type = genomic DNA
atggcagaagctagggatgtgttgaggtgtgtgtggtgggtgcttgtggtgcttcaattactggttcttactcagattgaggtgccaggtatttcggctgctgtgactgacacagggacaggaacggggactctgccgcctcggccgccgggcgcgtggaatgccacgtggacggaccgtctcaagaggaacttgctgctgaactacgacaagttcgctcgtccggcccagcacaccaacaccacggtcgtcaatatcggcatgaccttccgccacgtcgccctcgacgagctgaagtccatcatgacagtccaaggttgggtaaaaatgtcatggatagacgagaaactaaagtggaatgagagtgagtacggtgacctcggtatacttcacctggcaaaccacgaaatctggcagcctgatatagtgctatacaacagcgcgacgggcaacttagtggagtactacggaaacacccattgtctcgcgtacccaaatggggaagtgctgtgggtgccgccatcgcagttccaggttttctgcgacttggacatgcgtttgtggccttttgatacgcaatactgtcatctgaccctgggatcttggacctacgacggggaccacatcgatattcttctcgacaaatacggggctgatgtgcagctgatggagacgagttctgaatgggagtttatgggcgtggaggagacgcgcaacgtcaagacgtactcctgttgcgtcgaaccctacatcgacatcactttcaagctgacactgagacggcggtcccccacgtatagtgccatagtcataactcccgctacagtgattgtgcttctgacattggcaacattttggcttcccccaacagcaggggagaagatattattgaatggatgcatagcagtcatcatttgcatctttctcatgtacttttcacagaagttacctgctatggccggacacactcctcttgtggtgctgttctacagtagcagtctgtacatggtgtgtatcgccacgattatatctgtggtggtggtaaatctggcgcgcaaccaacacgtcactccccttccctgggtcatcaagaatctcctcacgggatggctaggtcaaatcttgggactaggccacataatagctcaggcatcagctttacatcacaatatgatgtcagggaacgcagaggagctgagagagcaacagacggctgtgtttgatgatcacgaccacatgggcaacagtagcgacgatcgtcagatgatcactccgcggtcccgcacctcgtcccagcaagactgggtcctcctggcggcggccatcgacagaatcactttcctcgtgtactgcctgctcttcgccatcctcgccatcgtgtacgcagtctaa

SEQ ID NO: 36 - DNA, 1635 bp, Periplaneta americana
  misc_feature 1..1635: note = beta1 sub-unit of the nicotinic acetylcholine receptor
  source 1..1635: mol_type = genomic DNA
atgaacacgggggcgcgagccctagcgcctgcaggcctcatccccgctttcctgctcttcttctggctactcgtgctgcctggttggtgctcggaggatgaagagcgacttgtgagggacctgttccgaggctacaacaagctcatccgccccgtgcaaaacatgacacagaaagtggatgttcgattcggtcttgccttcgttcagctcatcaatgtgaatgagaagaatcagatcatgaagtcaaacgtgtggcttcgattggtgtggagcgactatcaactgcagtgggatgaagcagactacggcggtatcggagtcctcagacttccgccggataaagtatggaaaccggacatagtgcttttcaacaatgcggatggcaactacgaagtgagatacaagtccaacgtgttgatctacccgaacggggaagttctatgggtacctccagccatttaccagagctcgtgcacgatcgacgtgacgtacttcccgttcgaccagcagacgtgcatcatgaagttcggctcgtggactttcaacggtgaccaagtgtccttagcgctgtacaataacaaaaactttgtggacctgtccgattactggaagtcgggtacatgggacattatcgaggttccggcctacctcaacatctacgagggcaatcatcccacagaaacggacattacgttctacataattatccgccgcaaaacactgttctacacggtgaatctcattcttcccacggtgctcatatccttcctgtgtgtgctagtgttctacctgccagcggaggcgggagagaaagtcaccctcggcatcagcatcctcctgtcactcgtcgtgttcctcttgctcgtctccaagatccttcctccgacatcgctggtgctgcccctgatcgccaagtacttgttgttcaccttcatcatgaatacggtaagtatcctcgtcaccgtcatcatcatcaactggaacttccgcgggcctcgcacccaccggatgccgccctggatccgcgccgtcttcctctactacctgcccatcgtgctgctcatgcggcgtccgaagaagacgcggctgcggtggatgatggagatgccgggcatgagcgccccgccgcacccctcgtacgggtcccccgccgaactgcccaagcacctcgcggtcagcagcaccaaccgcagcaagatggaggtgatggagctgtcggacctgcaccaccccaactgcaagatcaaccgcaagtcgagcacggagctcgcgggcggtggcggagggggcggtgggggaggaggtggaggcggcgggggaggaggaggaggaggcggcggtggcggcggcagcgggcgccgggaaagcgagagttccgattcgctactgctgtccccagaggcctccaaagccaccgaggccgtcgaattcatcgccgagcacctccggaacgaagaccagtacatccagatccgggaagactggaagtacgtcgcaatggtgatcgacaggctgcagctgtacatcttcttcttggtgacgacagcggggacgatcggcattctcatggacgcgccacatatattcgaatatgtggaccaggaccgaataatcgagatctatcgtgggaaatga

SEQ ID NO: 37 - DNA, 2322 bp, Periplaneta americana
  misc_feature 1..2322: note = beta2 sub-unit of the nicotinic acetylcholine receptor
  source 1..2322: mol_type = genomic DNA
ccgtcgctatggacatcaagcttgtgctcttagcattgtggctctacagcgatacttctcttgctcaagacggtggcattacagaagtgaaccctcacgactgcaagataatgttcacgtacggctatggatacaacaagtacttccatatatccgaatcaggaggtcattctggtgacaacatcacgattcgcttcctggtgcgggcccgcagcgacgcgcacctcctcttgtcatccgccccgtcgcctgtggaagggcaggcggtgtacgaggtggtgctgggtgcgggcaggaataccttctccgacatcaggcgtctgcgtcgcagttccaccaaggccacggctcgtacagtagatattctgtcgcctgtggagttgaggggcttttgggtgcatgtcagctacaagggcgacatacaggtgggcaaggaaggagacgacgtgccatttttgttctggacagacccgaccccgcttcccgtactctacttcagtttctgcacgtggacaggagtggtgggaaaatggttgtacgattgtccaatcgccaacgatacagagaaaatagaacttgtggaaccgaaacctgctacaatgactgaaaagctacgcaaagacttactttccaattacaatccgtacgcactgcctgtcttgcatgaggatcaccgcgttgcagttttcatgcatctgacttttcatcatgtgagactggacgtaaagcgctcagtgttttctgttgatggaataatgccaatgcattggatggatgagaaaatgcattggaaaccagaagactacggaggtttgaagtcagtacatgtaaatgaacatgaagtatgggtcccagaaattgttctttataatgctgttggtcatggagcaaatatactggggcatgcaggaatgacaataacgtctgatgggactgttatgtggtcgccatctgtgcaccttgaggcttggtgtaacctcaatcttgatcgatggcccaaggatgaacatacttgtgaactgcaattagggttttggtcgcaacagcaattcttagaactcattccaggaaatgacacagtgatggaagatgaacagcgaacaggatctgagtgggaagtgatcaaaatggagtcagagaccataatcactcagaatccatggagtcaagacgtcgacatcattgatttaacctcatctacatcaggtaccctcaccattcgcatcacagtaagacgtcaaaatcatccacacacgacaatgctggtgacaccattgttagtggtgagtgcgttgatattcctttcgttctggatgtcaccactgggagctgacagactttctgtgttgtgtgcatgtttggtgctgctctcacttttcacaataactattggaaatattttaccagctcctgctgagcatgttccttgcctagttcagatgtatagctggagcatggtagctggagtcttggcagttttgttatcaacgctggttatctgcataacacgctattcacaagcagttcctccccctgccattctgtgtgcattcatctcacttcgctttacacggttgttgttattgctgcctcaagtcaacacagaggcaggcaaaacgtacaaccatctacaggaaaattgtggcgaaaacgacacttgttcgattattactacaggaaatatgacaagttcgacaacgaagcaccaggaaattcagcagtattggataatacttggtaaagctattgatcgcatttcttgtttcatttatttcattttcctaatagtacttctctttaaatacaactaaacagactcatcttgaagatgataatacagatgtaaaatcagaatttgagatacatgaaactttcataaatcaacacgatattgcttttgtttgcacttctaggtgtgcactacataaaaactattaatcgtatgacattttatactgtcccaatgtgtagttatgtcgcagagtagtcgaaatgggggaaatcacgtgacaattacttacaacaggaccatttctttaaattattttaaacatttgtataatattacgtagaattctaatttcgtacagcaaatattttcaggaaaaagcctaacaccccagctaccaacctttacggaagggccagcagaaacggatgggggaaaccgagatgcgacatagcaactcggtaggacagtagtattctcgagttctgtatgtgtttctgtatattatgtaaatgtcaatacatgttatgagggataacactttaatatgtaaatcaggtttctatacagaaaataaatatctaaaaacacga

SEQ ID NO: 38 - DNA, 1442 bp, Periplaneta americana
  misc_feature 1..1442: note = beta3 sub-unit of the nicotinic acetylcholine receptor
  source 1..1442: mol_type = genomic DNA
aatgaccgttaagatcagcatagatgcggttgggggcgtgtactgagtagaggggcgtatgagtaagttatcggacaccaaaacgttgttgaatcgacgagtagcaacacgctatgtactggataaagattgtgttgctcttcttggccacgtgtctacatcaagggtggtgcgacggagagtgcaagagacaggctcccgtgacggacgaaactcgcctaaggaataaactgaagtgcggttatgacatcactgtgagaccgcaggacaacgagaaccataggaacagaacagaagtgttcctccacttactcctgagacacattatccttgatgaagaatatgacaccattggagtgtattttggactgtggatgtattggtatgatgaacggttgaagtggaatccatcagactacgggaatatcaagaatattcctgccgaaagcaacaccatatggatgccgcagattggatgggttaccatgaacatagtgagcgaccggctgtattcgccggacatcctgtgcaccttgttccacaatggtcacgtgagctgcctctccaggtcgtcgttccacacagtctgcaagtccgacctcactcactggccttacgactcccacgtgtgcgatgcgctcatggccccctggcggctcccggaggacgaactggagctcctcgcgtcgaacatgggtgacgagattttcagtatgtaccagccgaataaagaatgggcggtgctgaaggctgacataagaccccgcacggagaagtacgagaactctacatacgagtatctggaactggccttctcattggcccgacgctctgcgacctacgaggccatcgtcgtcattcctgcactcgtactcagcatgctgaccctggcctcctcgtggatcgaggcggagagtggggcacgtctgggactgcttagcagcatcctcgtctgccacatcctgtaccttcagttcattagccttctgctctacgagaatggaaacagctgtccactcatagtggtgttctatcgagactccttgatgatatgcgggtcctgcctcgttataacgctgcttgttcgatggacatcgtcccgttgtactgcgcctcccagctgggtgactgcggtcgctgagagagtgcaggaaaatcgaactggacagctgttgctcttcctaccacaacctcgcacgattgctgaaacaggagaagtggagcccgtctccgacctcggaggagcgacctggcatatcgctggcaagctgctggatcgagtcagtttcgtggtcttcagcgtcgtgtacttggtgctcttcattgtgtattattgctattatctgtgaattttgctctgtttctacttcatcagtgttgctgacttcactaacttctgttatgatataggcctagtcttattgaaaaattatacatctata

SEQ ID NO: 39 - DNA, 1508 bp, Periplaneta americana
  misc_feature 1..1508: note = beta4 sub-unit of the nicotinic acetylcholine receptor
  source 1..1508: mol_type = genomic DNA
gttccaacaagatatgacgggggcgtatccacaatggtgtcagatttatagatactccgttttccttctcagataccacgctttcagtgctttcaaatgttgccatcatatttgtgtatataattttctgttactggtgacacaatacataacaccattttaaaatgattggaaatatatttattctgtggatattttgtttagttactctgattgttacaggaaaggctgaatctaactgcaattctggctcgaaacccaagtctgcagaataccgcttgaagagagaccttctgtgcaattacgacaagacagtgagacctgttagagtgaagtctgatcaaacaagaattgcaattgacttgacacccatctccatgtactttgaagagagtgctgatgtgtttgttcttcatgcttcggtgaatctgatgtggagagatgaatttttggcttggaatatgtctgattatggcggtgtgaaaacaattctaatagacagcgattctatctgggtgcctgatgtgtatcacatgtcaacgtcatggtgggacttcacgtctctacggtcgcggtccaagctctgctccgtgaagtctcctgggttcgtgcactgctgggcggaccacgacctcttggccctgtgctacatagacctgaggaactggcctttcgacacgcagaactgcacggtgtcgctgtccaccctgatgcagtcctcggaggagatcgtgctgctatacatggagggatatttcaatttggagcaatacatccccaaccgtcagtggactctgttggacagaaagttcatgagcagcaacatgatgtatggagacaaagtataccctaatttacagttcagctttcttttgaagagaaacgcagccatttctgaagtcgccatcatcgttcccaccactgtgctcgttgtcttgaccctggcgtcgatctggttggggacagatcacgaagtccgtctcaatctgtgctgcacgttggtactagtgaactctctcgtcttgcaggatatcggaaacatgacagatggagacaaagcccctcagatagttctgttcctccgagactgcctcgtgatgtgcgtgttggctctggagcttgcggtgttcaaccagtgggccacaacgtcatcttcatctgtgccacgcgtcatcaatttcttccttgaaagcgacgcccgccagttgctgctaatgtcaagaaggctgagccacccgaagaattgcgagaaactggaagaagagaatgacgaagttatgaatgacactgttgccaaggagatgtcagctgaaaacaagagaaaatggaagcttttcggagaattgctagaccgaacatgtttcgtcgtgttcattttgacttattttgtgcttatctgtgtatacatattttaggacacgtgtatgattcctagagaaatggggtaataaacatattgttcgtagtttcgcgagttaaagctcattcacaatgaaaattaaacataa

SEQ ID NO: 40 - DNA, 1313 bp, Periplaneta americana
  misc_feature 1..1313: note = beta5 sub-unit of the nicotinic acetylcholine receptor
  source 1..1313: mol_type = genomic DNA
ggttaatgaactaatttgtagttccaaatggacttcgttcaagtttcgctgcctttcgttgtgtctaataatgcattacgttggtgtcattacaagtttatttgctgcgtttagttgctatggagtaacagcagaagacgtctgcagggtgtccagcccgaagagccagacgctacgcctgaagcacgatttgctgtgcaactacgacaagaccatcaggcctgtcgtcgacaatgggaatgccaccttcgtctacatcacaatgttactgaggagtctgacatatgttgaggagtaccacgttttggatattcactgttttctggtcatggaatggctggatgaacatttgctttggaagccttcggaatacgaagatctgaaacaagttctagtagagagtgaaaagatctggataccagatattgtgcactatggatcttctgcatgggaaagtttcaacaaggtagagaacacgaggtgtctggtgtatttcaacggtatggtcacgtgtgtgccgaggatggtgtattctacactctgcgacctggacttaacatattggcccttcgatagaattaactgctctgttagggcgggttctctgtcgcgtctagctggagaaataacggtcgacaacgattatggacacgtgaatctggtaaactatgaagataatcgagtctggaagttgttgtctgcaacttcggttgttcgggaacaagactatggagtcaatttcacatacagatggatagaatacaatttcgctatgttcagatactctgtgaaggaagaggtgattgctgtcgttcctgcactggctattgtcattctggtatctctttcgtgctggctttctccactggagaataaacgtatccacatctcttgcttcacgttagtctttcactgcctgtatcttcagtacatgggaggagtgttggggaatacaggagacgtcactccagttgcagtgcagttacaacgaaactcgttaattttattggcattgtctctagtcgtcgctatttttctgagatgcttgtctctctcatcaattgaacctcctacatggatacgtgggttgacaaaacgcttgttgagttatcgtatgggccaactgatactcctaatgaaaataacaaaggcttcaactattgccaaggaggaaggagcaaatgatgaagctgtcctcattgaaacatccgagacagataacgccacgacttggagtctgctgggaacgcttctcgatcgtgttttgttcaccatttgtgttattttttatgttttcatattcataagatgga

SEQ ID NO: 41 - DNA, 1627 bp, Periplaneta americana
  misc_feature 1..1627: note = beta6 sub-unit of the nicotinic acetylcholine receptor
  source 1..1627: mol_type = genomic DNA
gaaatatgttcccaatcctcagtaccgtttcatatttcccctcgaggaaaatacagtctgaaatcattactgtatatacatatacacagatacacacacacacaaactcattatctcctccttttgatactggcgtaatttgaacacggtgggcggtgaagtgttatgttacaagggagttcacacattcaatactgcccttccagtacgaagtcgtacatcaatacgtgaatattggtttagtatgcgttatctgaaaaatattacagtattgttgtatttattgagttacggtgtcgtgcttgctgaagatgactgcatcgaaaatccgacacggaagacggaaaccaccagattgaagcaggacctgttatgtgactacgacaagacggtgaggcccgtttctcatcactcttacgatacagtcgtcagtgtcaacatgttgctgaggagcatcagttttgacgaacatataaattccattaatattcactgctggatgttctggcaatggacagacgagcaccttcgctggaacccggatgattatggtggcatcacttctttaaatgtgaacagcgacgaaatctgggtcccagaagtcgcacagtttgctacgaccgccacttgggaggtggaggtgcggatgcccagtgggaggtgcgacatcaagtccaagggctacgtgatgtgcgcgccgggcaccgtgtacttcgccctgtgcgcccccgacctcacctactggcccttcgacaaggtgaactgctccttgagcctgggcgcgtggatgcaatccggggaggaaatcaacatcactatcgacgaggaggccgtggacttgttcaactacaaaccgagtcggcattggaatctgttatccgccactgcaaggaggcatgtggagagatacggggacaaccagacgttcccatggatccgctacagcttcgtgctgcagagacactccagcatgtaccaggccacagtgggtgtgccagctctgttgtacgcggttctggtgctgacgacgttctggctgcggcacgacggttgcaaccgcttgaacctctcctgcgtgactctgctctgccactacctgcagctgcagtacctgggctggatgttgcacagcaacggaaacaagtgtcccctcgtagtgcttttctaccgcgactcattggccttgtccggagtctctcttgtcttaaccatcatcttcagatatctgtcttccgtctccgcacctcctccagcttgggtgtctcgaactacaaactggtttacaagctttcgacttgggcagactcttctctcggtgaaagtatctgcagaaggtgccgctactgtaaaacccgagggaacggaggaggaagggtccggtttagtggagctccctcctgcagaggagtctagcggctggtctctgctcgtcaggctgctggatcgcacgtgtttcgcattatacgtcctggtgtatttgtttatttccatcagatgtttcacttaactcttttctcccaataaatttttaaactgtgtggcataatattcacagtctccgtcagaatgaaaaacaatttgttaattctacattgcgtcttaataactgtctgcaaaatcacgtaat

SEQ ID NO: 42 - DNA, 1655 bp, Periplaneta americana
  misc_feature 1..1655: note = beta7 sub-unit of the nicotinic acetylcholine receptor
  source 1..1655: mol_type = genomic DNA
tcttacataaatggtattcagttgtgtatcatcgttacagatcccttttaaatgctttattgtactgggcagcgaccatgctaaacttatggactactctgttaatatattcgttgctggttttgtttggggctcaaccttcttttcagcagtatcaacgtaaagacctgaaatgtgacaaagacataccagctaccactaccatcagcagacttcgaactaagctactttgctacggttacgatgccaaccgtccagcttactttggcaacgagactgtcggcgtctctcttcgccttcgtgttcttcggttcaattttgacgacaagacagacattcttgaaatacacagctggctgagagtggggtggcatgattttcgtcttaaatgggatgcagccaattacgacaacatcgataggatgcgagtgatcagctattacgtgtggaagcctgacgtgggagattacacagcagctgacatgggtaacgtgatcacgttaatacgacctgccttgtgcatggtgtactccgacgggtttgtgatatgcgtgcctccttacatccacacgattacctgttctgcagacctgaccttatggccttacgattcccacacgtgtaggctggaaatcggcgtactcttccacgaagaggacaagatacgcatcacggaagtagatttgtatctaaataatcccaacgtcaaccgtgagtggcaggtgacatccacgagttcttccatagagtcgaaggaggttccttcttttgaagacgtctattccagtggcgtttatacattcgtgttgaggaggcagccacggagtttcgcagctactgttatactgcctgctgttgtgttgatgttggtaaccctagcgacttactggatggaggctagtgagtcagagaggttgatactagcagctgtggatgttttgtgtcacttcgggtttttgatgcatctaggacgagtgcttccttcaaatggagacaagacgcctcttattgtgacgttttacagagattccatgttcctggcgactatggcagtatttacaactgtcattgttcgaggtcttaaaacggcatccgtagtgctaccaatttgggcatctgctttatccagatgggtcgttcagacacgagttggacaagtgttgatttttcataattcaaatcaaaatgtaaacacttctacatctggaaacgagggtacagcagtggataatgccaccaatgcgtcgttgccagactgggacgcattcttattagtgttcaactttttaacctttgtcgtgaccctgtttacatactttgtacttctggtgggttttataccgtaatatttgttctgcgacggtacaattcctaaaattaagaattttgttgaaaatatgttcaataaaagcgggtgagcaaattatctcccaaccggaagactgtaacttccgctatcggcaggagtacgctcccacaagctggttaaagagaaagcgcagttccggtactgatgtaaaatacagtgaaacctctcatttacggacaagaagggacgtaacaatctgtccgcatctgggaggtgtcctttattgggagggaggctccccaatctaacagtaaatttataatatgaattatttatcccttcacgc

SEQ ID NO: 43 - DNA, 1557 bp, Periplaneta americana
  misc_feature 1..1557: note = beta8 sub-unit of the nicotinic acetylcholine receptor
  source 1..1557: mol_type = genomic DNA
gaaaggaactatcattgtgtggattctatgttacataatgacttcacccgtgcattcttgtgtgtttattttatatttatttacagtgtatatttattgtgcacagtcagttgaggagacctgcacggtgcccaagacgaaaattcagacgcttcgtttgaagcaagatctgctttgtaactacgataagaagctgcgacccgctgacactcgtacaaagtcaaccaaagtcgtccctgacatgttcctgaaaagcctgacttatgatgacgaacacggcattttgaatgttcacagctttacactttggttctggaaagacgagcgattgagttggaagccgtcagaatacgagaatatcaattttttggttatgtcaactgatgaaatatgggtaccggaagtggcacgtttggttccaagcatgatgcggaacatggacaaggagacaaccggcatcggatgttacgtgtactcgaacggctacgtggcttgttcgatggttgcggtgtatgctgcagtctgtgccttggactccacttactggccttacgacagtctgaactgctccttccggcttggatcatggcgggaggctggcgatgatatttcgctctctctttataataaacgggtacaccagagtaaatacgaaccaaatgagtggaagctgttgtctgcaacctcgacaatacgcttggatctattggaacacaacgcaactttaccatgggtagagtacaggttcacattgcagagacactctttcttacctgaggtgatcgtgggtgctccggtcttagttaatgcaatcatggttttactcaacttcgtgtttctttctgcgtcggatgataaacgaatcaccttctcttgtgtcattttaatatgtcactgcatctatctgcagtatttgggaataactctgggaaacactggagagagcagcccgattgtggtgcgactgtaccgtgattctttggtattactaggcgtgtctctcgtagtgacaattgctctcagatgtttgtcctcctcgacagatcctccaacatgcatacagaatcttacaaaccgtgccttgagctacagagtgggccagttggttgtcctcatgcaaatatcacgtcaggttacggccgttgctaaggacgagacagatgaagacgatggtctcattgtgacgtcatcaccagatagagaccgtgtttgggtcttgttctgcaaaatagtggatcgtatttgtttcgttgtgtatagtctgtggtacttggtcctattccttagatggactgcttgatgggaactcttacgaaatactagtacaagtttatctcggctgtgatctgagtgttaagaattactttactccattagtatacctaccgaaaagatctagaacacatttgaagtagaataaattgatttaaattctgaaatcctaaacaaatttaatattctgagtttctagaatcaccttcgttagtcaacctcttttgtgtgatgtaagtttaattcctaaaggatttatatttctgttctataaaatgtacctaa

SEQ ID NO: 44 - DNA, 1679 bp, Periplaneta americana
  misc_feature 1..1679: note = beta9 sub-unit of the nicotinic acetylcholine receptor
  source 1..1679: mol_type = genomic DNA
tattacacgatgatgatcgccggtgattgctcctgaaacaagttcaggacgatttgtgtcgcgtaccgatcaattagagtaggtcatgttcccagaacgtcacaaaaatggccgtgctcaagcattcctgaatttgagctgttatggattataacatcagatatcatcatgatgatcatctaatggcctgatacaggtctccaaccgtcttttgtgagatcttccaacggatcttttcccgcgtgggacccagccgtccgtccaacagtaccggtatcaacgcaatgacccgaaatgtgacaaagacatgccaggtaccattaccgtcagccggcttcgaaggaagttattttgcttcggctacgacgtcaaccgaccagcttataacggcaacgagactgtcgacgtatctcttcatcttcaagttcttcgattcaattttgacgagaagacagactctcttgaaatacacagcatcttgattgagaggtggcgtgattttcgtcttaaatggaacccagccgattttgataacattgataagctgttagtaatcaactattacttgtggaggcctgatctgggagacttcaacgcggctgacatgggcagaatgtcggtgtacggagttcctaccatgtgcatattgcaacctgacgggattgtgatatgcgtgcccgtcactacttacacgacttcctgctcagcagacctgactttgtggccctacgattcccacacgtgtaatctggcggttggttcctggagccatgaagaggacaagatcaacatttcggcagcaatattgtcgtttccaagcgacgcaaacctgaaccgcgagtggcaggtgacatctacgagttcttccgtagtgtcacagaagataccttcgtttccggacgtctactccacagccgtgtttacattcgtgttgaagaggcatccaaagagcttcgccgctactgttatactgcctgctgtcgtgttgatgttggtgactctggtaacttactggatggaggctagtgagtcagagaggttgatacttgcagctgtggatgttgtctgtcacttcacattcttgatgcatctaggacgagtacttccttcaaatggagagaaaactccccttatcgtgacattctacagagattccatgttcctggcgactatggcagtatttactgcagccattgttcgaggtcttaagacagtatccgtagtgctaccaatttgggcatcggccttatccagatgggttgttcagacacgagttggacaagtgttgatttttcataattcaaatcagaaggtaaatacttcagcctctgaaagcgagagtgcaacagtggataacgccactaatccctcgttgccagactgggacgcgttcttgtccctgttcgatttgttatcctttgccgtgactttgttaacatactttgtacttctggtgggatttataccgtaattttcaatccgcgactctatgaattctcaaattaaaacttatgaatttagttaagagaattttaataaattacaacttctgcttttccatgaattgttagtggttggaaaaatgtatcatttattgttcagctatttactgaataaactgccgtcttcaattctactgcatgtaggttattttattagaaaaaattgaactataagta

SEQ ID NO: 45 - DNA, 1284 bp, Periplaneta americana
  misc_feature 1..1284: note = beta10 sub-unit of the nicotinic acetylcholine receptor
  source 1..1284: mol_type = genomic DNA
cacggcagctacagtcgaattcatttctgtcgttcgcagcgtagctatggggaaaatttctgttctgttacgaatactgatagtgatcttcctggccggcctcactcgagcgcaggacgacccgtgccccaactccacctacatctccaacttgtctcgactgaagagctacgtgtactgctcgtacgacagatggatccgtccagttcgacagaacaacgaagcggtccctgttaagttgtggttcgttctgaagaggattgacttcgatgacaagaaggatatattgaaagtattcaacatatttgtcatgatatggacagatgagcacatgaagtgggacccatcaaaattcggtgacataaaagattttcgcgtcagtagttacagcgtgtggacacctgacatcggcgtttatacctctgccgaccagactgcattcaacccgtacctacggccctctgtatgcagggtgtccaacacaggggaagtgttatgcgctcctcctgtcacgcatacgaccacctgtgcagccgatctgaggcgctggccttacgacacgcacaagtgtggcctggtgttggggtcctggagccacagtggcgaggaactcaatctctccctgagagagcccagtatttatcttggtcagtacaacatgaaccgagagtggcacttggtttcaatgagggcgtacaaggaggttgaaacgtacggcagcaacaacacgtatccgtcggtcacctacgagttcgtcctgcagaggcattcgggaagctacgctgccatagctgtaatgccagcagttgtgctgatgttgctgacgctgctgacgtactggatgaaggcggaccacgctgaccggctgattctctctctcctcagcctgttttctcacttcgtcttcctgcagtacctgggcaacatgctgccgtccaacggcgaccaagttcctcttctgatgacattctaccgagactctatgctactggccaccactgccctgatggctaccctcgttatgcgggggctgggaaggaactcactggccgctcctgtctgggtgtccgatctgtccagcagggtgttacagaaccgtttcggacaagttttggttttccacagcttggatccaaaggccgctgcagccgtgtcagaggcgtcgggaggggagggcgccggcctagtggagggagcagctgcaacgtcggggtggaacaccttcacgacgctactcaacctccttctcttcatccttacattgttaacctacttcatactctt

SEQ ID NO: 46 - DNA, 20 bp, synthetic construct
  misc_feature 1..20: note = beta2-ORF Sense
  source 1..20: mol_type = other DNA
agcattctgactacagcgag

SEQ ID NO: 47 - DNA, 29 bp, synthetic construct
  misc_feature 1..29: note = beta2-ORF Antisense
  source 1..29: mol_type = other DNA
aactagtagatagatacacaaacattgat

SEQ ID NO: 48 - DNA, 40 bp, synthetic construct
  misc_feature 1..40: note = beta2-dsRNA Sense
  source 1..40: mol_type = other DNA
taatacgactcactatagggtagatgctgcaataacagac

SEQ ID NO: 49 - DNA, 40 bp, synthetic construct
  misc_feature 1..40: note = beta2-dsRNA Antisense
  source 1..40: mol_type = other DNA
taatacgactcactatagggagtgtctatcgtagaacctc

SEQ ID NO: 50 - DNA, 40 bp, synthetic construct
  misc_feature 1..40: note = LacZ-dsRNA Sense
  source 1..40: mol_type = other DNA
taatacgactcactatagggatgaccatgattacgccaag

SEQ ID NO: 51 - DNA, 40 bp, synthetic construct
  misc_feature 1..40: note = LacZ-dsRNA Antisense
  source 1..40: mol_type = other DNA
taatacgactcactatagggtggcggccgttactagtgga

SEQ ID NO: 52 - DNA, 20 bp, synthetic construct
  misc_feature 1..20: note = beta2-qPCR Sense
  source 1..20: mol_type = other DNA
cgcaaagacgaagagtcgag

SEQ ID NO: 53 - DNA, 20 bp, synthetic construct
  misc_feature 1..20: note = beta2-qPCR Antisense
  source 1..20: mol_type = other DNA
acggatagcgtcaggaacac

SEQ ID NO: 54 - DNA, 20 bp, synthetic construct
  misc_feature 1..20: note = RPL7-qPCR Sense
  source 1..20: mol_type = other DNA
acgtaaagagcgcgtgaaga

SEQ ID NO: 55 - DNA, 21 bp, synthetic construct
  misc_feature 1..21: note = RPL7-qPCR Antisense
  source 1..21: mol_type = other DNA
ggttcacaccacgaatacgca

SEQ ID NO: 56 - protein, 420 aa, Acyrthosiphon pisum
  REGION 1..420: note = beta2 protein sequence
MFKSFLKKVLCLIIIIKFANIDAAITDNYGKQHWNDTWTDKLRRDLLRFYDRHSRPVISDHLTNVTVNTVLKYVDLDFQNSIMTINAWVSMTWQDEKLLWNVSDYGDTKVVNMAYDEIWMPDIVAYNSADGHHDETYGKINCVVRSDGMVHWIPPAKFRVFCDVDLTNWPFGEHTCSLRLGSWTYDGNSVSMQINQKINTADMINLEEGCKWDITKVTQRRRVESYSCNSNDIYEDIHYSITVKRKIPAYHSIVVIPAIVVVFLTLSVFWLPPESTEKFMLSGVTLVIVCILLMMISGNVPIMSDRIPLIVMFYSNSLGMVTLSMVFSVLLHNLAKYKQPDHKVPKYITQLLNTKFGTVMRFTSMKNDDEDKILDESNDDKSQYPQLKESQWYYLALILDRTMFIVYLFLFIAMVIYFLK

SEQ ID NO: 57 - DNA, 40 bp, synthetic construct
  misc_feature 1..40: note = beta1-start Sense
  source 1..40: mol_type = other DNA
taatacgactcactatagggatgaacacgggggcgcgagc

SEQ ID NO: 58 - DNA, 40 bp, synthetic construct
  misc_feature 1..40: note = beta1-start Antisense
  source 1..40: mol_type = other DNA
taatacgactcactatagggcatcctccgagcaccaacca

SEQ ID NO: 59 - DNA, 40 bp, synthetic construct
  misc_feature 1..40: note = beta1-end Sense
  source 1..40: mol_type = other DNA
taatacgactcactatagggccgccgcacccctcgtacgg

SEQ ID NO: 60 - DNA, 40 bp, synthetic construct
  misc_feature 1..40: note = beta1-end Antisense
  source 1..40: mol_type = other DNA
taatacgactcactatagggggtccgacagctccatcacc

SEQ ID NO: 61 - DNA, 40 bp, synthetic construct; identical to SEQ ID NO: 50
  misc_feature 1..40: note = LacZ Sense
  source 1..40: mol_type = other DNA

SEQ ID NO: 62 - DNA, 40 bp, synthetic construct; identical to SEQ ID NO: 51
  misc_feature 1..40: note = LacZ Antisense
  source 1..40: mol_type = other DNA

SEQ ID NO: 63 - DNA, 19 bp, synthetic construct
  misc_feature 1..19: note = beta1 Sense
  source 1..19: mol_type = other DNA
ggtgaccaagtgtccttag

SEQ ID NO: 64 - DNA, 19 bp, synthetic construct
  misc_feature 1..19: note = beta1 Antisense
  source 1..19: mol_type = other DNA
atgattgccctcgtagatg

SEQ ID NO: 65 - DNA, 20 bp, synthetic construct
  misc_feature 1..20: note = Actin Sense
  source 1..20: mol_type = other DNA
gactactggtattgtgctgg

SEQ ID NO: 66 - DNA, 20 bp, synthetic construct
  misc_feature 1..20: note = Actin Antisense
  source 1..20: mol_type = other DNA
aaagctgtaaccacgctcag

SEQ ID NO: 67 - DNA, 100 bp, synthetic construct
  misc_feature 1..100: note = "start" fragment of the beta1 nicotinic sub-unit of Periplaneta americana
  source 1..100: mol_type = other DNA
atgaacacgggggcgcgagccctagcgcctgcaggcctcatccccgctttcctgctcttcttctggctactcgtgctgcctggttggtgctcggaggatg

SEQ ID NO: 68 - DNA, 100 bp, synthetic construct
  misc_feature 1..100: note = "end" fragment of the beta1 nicotinic sub-unit of Periplaneta americana
  source 1..100: mol_type = other DNA
ccgccgcacccctcgtacgggtcccccgccgaactgcccaagcacctcgcggtcagcagcaccaaccgcagcaagatggaggtgatggagctgtcggacc

SEQ ID NO: 69 - DNA, 20 bp, synthetic construct
  misc_feature 1..20: note = T7 promotor
  source 1..20: mol_type = other DNA
taatacgactcactataggg

SEQ ID NO: 70 - DNA, 1820 bp, Acyrthosiphon pisum
  misc_feature 1..1820: note = Acyrthosiphon pisum resistance to inhibitors of cholinesterase protein 3, transcript variant X3
  source 1..1820: mol_type = genomic DNA
actaataattcctaattttgtttttatccctccgattgctcagccacagtcctaatattatttataggattttctttaaatttcacatctgtgtggttgcacgatcgcgttaaaatgccgaaaattggttgacgaagttcggcataatattccgttttaggaaaattgaagataataacattgacagacatggcgacagagataagtcaaagcaaaacaatctgcattctggctgttgtagcaggatgtttcgccattctatggccatctttattttatccaatgctaaaaggatcatttgcacctcagactcacacagataatattccgcaagaaagaactcctgttcacttaagggcctccaaagcaacaatgcatccagcattaatggaaaaaggacgagcaattccccaacctcatattggacctagacatagtagtccaactcctcacagtttcaaaccaccaattccaggatcaagaccgacaatgggcggtatgggcggcaccagtaataaaatcgctggaggagatggtggtggtgccatgaatatactaatgccaatgtatacaattggaatacttcttttttttgtgtatactatgatgaagttgttatcaaaaaaagacgacgttgaagatactattgatgataaatattcttcacattatccttatggcagagatagtaaatcaaagctacagccgcagaatcttcctacaaaattagggttcattgaaaaggatacgagggatttagaaatagatgcgctccgtcgcaagctagaagaaacagaggctgcgatggaacgcattgttaagcaaatgatattggcgtctgagtgcaatgggatggatgggaataaggagtttaaaaacgaaaatattgatgagggggaaagtccgacaaatacgttatccaatgacaacgtaaatgatacgattactactaaaaagtctacaccatcagaggaatcaaaaagcattaaatctttaaacacttccgaactatacgatgaaaatactacagattccgaaatttccgtaaaaaaaatcattcaagtggtcaatatggaaacatctgaaagtgttggtggtggtcattgttggactccgactgaaaaagagaaaataccttctaaatgtaaaacagcatctatagaagagcccatttctctactcattcctggtatgattcctaagaactctcaagttttaataagcgatggtccactcaatactccctcagaaactgaggatcacgaagcagtaatttcaagcaaagttacattatcattaatacctgacgaccgagtagaggaatacctaactggtgatgaagcagaagatgataatactacttcaacactaccaaacattaactaatatggcaaaaaatgaaaacataaacgtatgatttacctatatttaataattatatgtcgagtaaatatattcaatgtacatgcacgattttaaaataggtaatgtttggggatttgacgagagtaataatatgggaataggaccgtatattatgaaagtttttaattgcacatttactatgcacttaaatattttgaggtagattttaatgttataactttatgaatgagccattataatgaacttttcaatgtatgttaacaaatatgtgatcagtattattttatgtatacctagtttgtcaatttataagtacatttttttgttacttttgaagtactttagactgataatgttgccaaatgtttagcaaatatttcttgaatttattgtactttacctgtaataaatatctgatgtgtcaaatcaaaaaa

SEQ ID NO: 71 - DNA, 2114 bp, Acyrthosiphon pisum
  misc_feature 1..2114: note = Acyrthosiphon pisum resistance to inhibitors of cholinesterase protein 3, transcript variant X2
  source 1..2114: mol_type = genomic DNA
actaataattcctaattttgtttttatccctccgattgctcagccacagtcctaatattatttataggattttctttaaatttcacatctgtgtggttgcacgatcgcgttaaaatgccgaaaattggttgacgaagttcggcataatattccgttttaggaaaattgaagataataacattgacagacatggcgacagagataagtcaaagcaaaacaatctgcattctggctgttgtagcaggatgtttcgccattctatggccatctttattttatccaatgctaaaaggatcatttgcacctcagactcacacaggctgttgcaatgtgctgtccagtacggacgtgaatatagttaaaatagtaatggatatatgtgatcgtgtaattgatacaacggacagaaaaacaaaattggaaaaatgtcgtcacgatattttcaacacatgtgctatcaatataactgatttaatcaatgaaaataaggttggcaaaatttcgcacaataaacatttaatcgacgaaattagatctttgaatggatctctatgcctaaaatatcattatggagtttccttgacaagcttgggggttacacatagacttaaagaaagcgattctttaatgaataatattccgcaagaaagaactcctgttcacttaagggcctccaaagcaacaatgcatccagcattaatggaaaaaggacgagcaattccccaacctcatattggacctagacatagtagtccaactcctcacagtttcaaaccaccaattccaggatcaagaccgacaatgggcggtatgggcggcaccagtaataaaatcgctggaggagatggtggtggtgccatgaatatactaatgccaatgtatacaattggaatacttcttttttttgtgtatactatgatgaagttgttatcaaaaaaagacgacgttgaagatactattgatgataaatattcttcacattatccttatggcagagatagtaaatcaaagctacagccgcagaatcttcctacaaaattagggttcattgaaaaggatacgatcgtgaacgcgttttcgtctctattggaagacgtgaatcgagaaatattatcgtctatagttgaaaaagaaaatgttcccacaataattgaaaacgaggagtttaaaaacgaaaatattgatgagggggaaagtccgacaaatacgttatccaatgacaacgtaaatgatacgattactactaaaaagtctacaccatcagaggaatcaaaaagcattaaatctttaaacacttccgaactatacgatgaaaatactacagattccgaaatttccgtaaaaaaaatcattcaagtggtcaatatggaaacatctgaaagtgttggtggtggtcattgttggactccgactgaaaaagagaaaataccttctaaatgtaaaacagcatctatagaagagcccatttctctactcattcctggtatgattcctaagaactctcaagttttaataagcgatggtccactcaatactccctcagaaactgaggatcacgaagcagtaatttcaagcaaagttacattatcattaatacctgacgaccgagtagaggaatacctaactggtgatgaagcagaagatgataatactacttcaacactaccaaacattaactaatatggcaaaaaatgaaaacataaacgtatgatttacctatatttaataattatatgtcgagtaaatatattcaatgtacatgcacgattttaaaataggtaatgtttggggatttgacgagagtaataatatgggaataggaccgtatattatgaaagtttttaattgcacatttactatgcacttaaatattttgaggtagattttaatgttataactttatgaatgagccattataatgaacttttcaatgtatgttaacaaatatgtgatcagtattattttatgtatacctagtttgtcaatttataagtacatttttttgttacttttgaagtactttagactgataatgttgccaaatgtttagcaaatatttcttgaatttattgtactttacctgtaataaatatctgatgtgtcaaatcaaaaaa

SEQ ID NO: 72 - DNA, 2132 bp, Acyrthosiphon pisum
  misc_feature 1..2132: note = Acyrthosiphon pisum resistance to inhibitors of cholinesterase protein 3, transcript variant X1
  source 1..2132: mol_type = genomic DNA
actaataattcctaattttgtttttatccctccgattgctcagccacagtcctaatattatttataggattttctttaaatttcacatctgtgtggttgcacgatcgcgttaaaatgccgaaaattggttgacgaagttcggcataatattccgttttaggaaaattgaagataataacattgacagacatggcgacagagataagtcaaagcaaaacaatctgcattctggctgttgtagcaggatgtttcgccattctatggccatctttattttatccaatgctaaaaggatcatttgcacctcagactcacacaggctgttgcaatgtgctgtccagtacggacgtgaatatagttaaaatagtaatggatatatgtgatcgtgtaattgatacaacggacagaaaaacaaaattggaaaaatgtcgtcacgatattttcaacacatgtgctatcaatataactgatttaatcaatgaaaataaggttggcaaaatttcgcacaataaacatttaatcgacgaaattagatctttgaatggatctctatgcctaaaatatcattatggagtttccttgacaagcttgggggttacacatagacttaaagaaagcgattctttaatgaataatattccgcaagaaagaactcctgttcacttaagggcctccaaagcaacaatgcatccagcattaatggaaaaaggacgagcaattccccaacctcatattggacctagacatagtagtccaactcctcacagtttcaaaccaccaattccaggatcaagaccgacaatgggcggtatgggcggcaccagtaataaaatcgctggaggagatggtggtggtgccatgaatatactaatgccaatgtatacaattggaatacttcttttttttgtgtatactatgatgaagttgttatcaaaaaaagacgacgttgaagatactattgatgataaatattcttcacattatccttatggcagagatagtaaatcaaagctacagccgcagaatcttcctacaaaattagggttcattgaaaaggatacgagggatttagaaatagatgcgctccgtcgcaagctagaagaaacagaggctgcgatggaacgcattgttaagcaaatgatattggcgtctgagtgcaatgggatggatgggaataaggagtttaaaaacgaaaatattgatgagggggaaagtccgacaaatacgttatccaatgacaacgtaaatgatacgattactactaaaaagtctacaccatcagaggaatcaaaaagcattaaatctttaaacacttccgaactatacgatgaaaatactacagattccgaaatttccgtaaaaaaaatcattcaagtggtcaatatggaaacatctgaaagtgttggtggtggtcattgttggactccgactgaaaaagagaaaataccttctaaatgtaaaacagcatctatagaagagcccatttctctactcattcctggtatgattcctaagaactctcaagttttaataagcgatggtccactcaatactccctcagaaactgaggatcacgaagcagtaatttcaagcaaagttacattatcattaatacctgacgaccgagtagaggaatacctaactggtgatgaagcagaagatgataatactacttcaacactaccaaacattaactaatatggcaaaaaatgaaaacataaacgtatgatttacctatatttaataattatatgtcgagtaaatatattcaatgtacatgcacgattttaaaataggtaatgtttggggatttgacgagagtaataatatgggaataggaccgtatattatgaaagtttttaattgcacatttactatgcacttaaatattttgaggtagattttaatgttataactttatgaatgagccattataatgaacttttcaatgtatgttaacaaatatgtgatcagtattattttatgtatacctagtttgtcaatttataagtacatttttttgttacttttgaagtactttagactgataatgttgccaaatgtttagcaaatatttcttgaatttattgtactttacctgtaataaatatctgatgtgtcaaatcaaaaaa

SEQ ID NO: 73 - DNA, 1137 bp, Acyrthosiphon pisum
  misc_feature 1..1137: note = Acyrthosiphon pisum CG17218-like
  source 1..1137: mol_type = genomic DNA
gagtcttgcacagtgctccgaagtgtggcctattctcaacattaattgtccaaaaattctgaataaaacacacttttttttacattctccaggaatattatagtataccgtctggtcttataatttctattaatccattaccatatacaataatgatccgttgtcagatgtacgcgatggttgtcttcgcattcgggtgtctcgtcttgttaccggactacggcaaagcaatcaactgttgggtgtgctccacggacacggaccaacgttgcaacgacccgatgaacatgaccaaaggtgcaatcgaggactgtagcagagcaccgcattcagctttcttacagcccgtgtgcaagaaacaaaaacaaagagtgaacggcgagctactgataatccggtcgtgcgcttgggcgtcggacagtcggagcgacgacggcccatgcgccataaacactccagccaacatacgcatcgaacactgcagcacatgcgaccaggacctttgcaacggcgcagtggtcatcggaggttgcaccgtgttcctgaccatggtgttggcggttgcggtccatctcgctacctattaacgcggtaacacccctcctaatgatttttttcttctctgaaacacgcccttttccgccccaacaaaacacagtcatgtctacgaaaatcacgtaggtacctttatgacacacatttatataggtatatataatatatactgtttgttcaactatataatatatattttatacattttatacctaacaataatttttatttattccaagacacattttacacttcaacataaagctattacatacaccaaacgcggcaagtcataacacatttatatatatttttatgtatataaaatatattaggtacattttacgtgtgtatgatttcttcgatattaaatacctctctttttataattattgtacctatatataaagttaaggaaagtatataggtaattacgtttaattttaataatataattttaattataatattattttgattgatatacctaatatatctcaactaccaacctatcgataagaaattgtaaatggttattaagaattacatttttatgattatttaccaaaaaaaaaaaaaaaa

SEQ ID NO: 74 - DNA, 1795 bp, Acyrthosiphon pisum
  misc_feature 1..1795: note = Acyrthosiphon pisum protein quiver
  source 1..1795: mol_type = genomic DNA
ggcgcccgaccgtcatacgcgcagcaaactttatattgtacgctatcaccgtcgcggcgccgtcgtcgaacataatattattcgtccgccggtacaccctgttccgtggtagtatacgcgcgcgcgtttataaatacacacacacacacacacacacgagcgcgcggaagtttgtaaacacgatattattattacgatatttattagattatattatatattattattatcatattttaagtgtattgcaaaacagtcgcgtacacacacacacacacacacacacacgaatattcatatttatcatacacacgcagtgtacgcacacatcataccacaagcaagtgcatttaattatatccgatattgttccgccggaagtatgaatggcgctaaatgcgccgcgtgcctagccgccgttctgttcgtacttttcgcgttcgtcgtcaaaggtgcaaaatcgataatatgctacaagtgcaattctaaatatgatccgaggtgtggagatccattcaatccgtacaccattggtttagttgattgcaacataacagacgtgcctagtcatttgaaaggaaaagaaccagtgatatgtcggaagactgtccaaaacattcacaagagtattcggatagtgcgggattgcgggtacgtggatgatcctgctcacgatgatcaggattgctatggtagactcgggtcacatgacatcgacgtgacccattgctcctgtaccacggacatgtgcaacggttccaaggatgtcgtttccaatggaccgtactggtacagcgcgaccgccacagcgatggtagctacagccgccgtcaccataatggggttcttccttaacatacaccatttatagacgatatttacgagatcggacccgatcgaagtttaacgagagataaaattaacaaaaaaagtcattttaaataacaaacgctttataacatagcccaaacagactatatattatatattgaaggtgtcactcgacgacaatatgaggacataagttcacgttgtatatttatgataatttttattattattatattatgctcagagtgcgggaaaacgttacgaaatcgccaaactataatattctaagagctgcacgttaaagacgataatatagacacggaaaacagcgcataaacttttatgatattataaacccgcacgctgatctagtctgatatgtcttaacccatataatatatatatatatatatatataaatatacagtattcgagcggtatcttcatattttattatttttaagaaaaaaaaacgataaatatgtattttattttatttctgttgagatgcgtgttttttaatagctcaattaagtaaaaagttttttcgcactataagttataacatgtataagaacaataaattgtgaatttatattatctcagaaaactctcataatactattttaattagacttatctttgtttttaagatgaattcaaaccgtttattaaactgtttattttcgtgttacacaaaaagatccgttatggtacataaattcacttacgacacgcgaattatatttttatattacattatcgtagttataatgttagaaaatcgttttggctaaaccgtatttttaattagagaaaagccgaattggaaaccgacaatgaactgtacctacacgataacaatctcaacatgtgcatatgcaaagcccgtcatttgatttttacataatgttactcgtgttaagcattttatctttgaataaactattataaaaatatataa

SEQ ID NO: 75 - DNA, 812 bp, Acyrthosiphon pisum
  misc_feature 1..812: note = Acyrthosiphon pisum uncharacterized LOC100164124
  source 1..812: mol_type = genomic DNA
tgggcgtgttatttaaagttaaactgaaattgtccgcaagaagtacagagttatagtattatacattaaattacattaaatataagcatgttaatcaatccaataatgtaaactttttaattgagttcttttaattaatttgtcaaaatacaaatctcgttatgatgccgtccgctctttgtgtgttttgcatgagcttcattgttttgtacagcgtttattctgtatgtggtctcatgtgttggcattgtgattcgcttcatgaccccaaatgcgtggatccatttgataatacttcgatgccaatgaaagattgcaaactggaaaaactggacacatatcccggtgtgcgaggtacaatgtgccgcaaaattcgtcaaaaagtgtatggaaaatggcggttttaccggagttgtgcttttcttggcgaacctggcattggtggtgacgaacgttattgtttaatgaggacaggcacacataatatatttactgaatattgtacttgcaattctagagatggatgtaatggtgcatctggcatttcttcttcacttttaagtatatcgttagcaccagctcttattgtttacagaagactgctgtaatgtgttcatccaacaataaaagtacttgatatttgaataactttttaagattaaagaatatagttttcatcagatatcaaaccattttttttaaggcaacaataaagttaactattttatttttaaagccttcgaaattgttcaacttgttatgatatattttgttgtgataaataaactgaaaagtgtaactgatccgtccat

SEQ ID NO: 76 - DNA, 1229 bp, Acyrthosiphon pisum
  misc_feature 1..1229: note = Acyrthosiphon pisum CG7781-like (LOC100160953)
  source 1..1229: mol_type = genomic DNA
gagtgcgccgcccgcgattgcagattataccagcggcgtagactcggcgctgccccggggcccctgtgttgcgctcgaccgtcccgacacgcaccatgtcctgcaccgtcacgtccgtcgcttacgccacgatagcgttgatcgccgtctcgattgttttcaacacacacaccgtgaaggcacaagctgagatacggtgttatgtgtgcaatagtcataatgacacagattgtgctcaagaaaaacttccagatcattttaaaacagaatgctcaaaattaaatataggtgttccagaggatagtcgcaaaaattataccctctgcagaaagataatacaaacaatcgagccggaggtcaacggattgccgtcagatagaaatcggatcattaggacatgtggctgggacgcttcaaattacaataataaatgttaccatcgatccggatttggtggcaaacaggacgtgtgcacttgtatgacagattactgtaactcggcgcaaaagacatacatagcgtttgtaactgtagtggcagcagcattcgctatccttattttgtagaaacgaggatattataactctcaaacacgagacaaatatttttaatttaagcatggatgcaagtattgttaataaatgttgagaagtatataaataaaaccagcaactggtaccttcatgacaaaaaataaacaatactgtttttattagttgaaactttggtagcatatagatacaattaggtataagttagttaggtatcacactatttttctataataaaatattttataaaatatgtgtgttttcatagtgtgcaaagtattaaattgtatagtctatactctataatatgaattataagtagttgtacaaaagagttgtaaaactgtccaatgaacctgcattatacataccttgataaacataaaatactaaagtaaaattcttctttcaaacttttttagttccagcgtaaaaatgtaaatgttaaacctgaaaacaattgtgtattatataatgtaatttatgaagtatgtagatacaactttctagatctctagtaaagatgtatgttaaaaataataggtggatatttttattataattattgtttatttagtttatatttcttatacataatacctacctacgcaattagatttgtttgtaatgttttgtttgataacattataagtattaaacctcaaaaaaaaaaaaaaa

SEQ ID NO: 77 - DNA, 968 bp, Acyrthosiphon pisum
  misc_feature 1..968: note = Acyrthosiphon pisum quiver (Qvr)
  source 1..968: mol_type = genomic DNA
agttttaccgtatagtcatcgcgtacccgtcgtactaagtttgtcgcaacgcgttcgcgcacaaaggtataaccacggttattgtattcggattggttcggttcggttcgttataatactatcatctgtctatatattttgtttcgccgggatgtagagttttcgcacagcggcggacagacgtaatattataatattatatccgccgggcccgtcgaagaaggggcgtcatcctgccaccaccgctaacaccaccactaacgccaccgcctcccaacgtcgtcagcagtactccagagcggaaaatgcggtggacaattgcagttgtcgtgttggttaatatatcgctgtcccacgaggaatgttttccccctcggtccatttactgttacgagtgcgacagcgccaaagatccacgctgcaaagatcctttcaactacacagccatgcctcaagatcaaccgccactggtcagctgcaacggctgttgcgttaaaatggtccggtacgtgcgaacttcatacgagagcatcaagagaacgtgcacatcacaactgcaaatcaacctgttcatggttgaccacgtgtgcatgatggaaggaacgggtacgggacacatgtgtttctgtgaggaggacaactgcaacgcggctaccggaccggcacgatcatcggcgttagtgtccggtgccatagccctcgcaatgtacgcgcttaaactactgttgtaccggtagacctggtccaatagcatttcatcacacatttatttacaatttttattttccctaagtgtatatttttatttttattttttatcatttcacttaaatctatacgtatgaagtaataactaataataataaaaataacaataatgtcattaatgtcgaaatatgttaacaatacaagagccctagttgacagaataaaaactcaaaagccattgttataaaaaaaaaaaaaaa

SEQ ID NO: 78 - DNA, 1197 bp, Acyrthosiphon pisum
  misc_feature 1..1197: note = Acyrthosiphon pisum transmembrane protein 35 (Tmem35)
  source 1..1197: mol_type = genomic DNA
gtaaagcactaaagcgtaggaggagtccctgcggattgtagatcgtttggactgactaaatttttttcccataaaatttaaattcgttttggtagctgctgtgtagtaattttaatatagataattataaatccagatatgggatcaatagttttaaagagtctttcggttctacttggtatttttttcctatttgtaggtactctgaaaatgagtcctgtcatcagtaaagagctgcacaaggatctccgaaaggactatgttaagtatgccaaagtatttccactctccaaaatgatcgactttaaagtgccagccaaatggtacagaagagctgtcggtggtactgaagtattaagtggtgtttgcttggcatttgttccttatagaaacgttaaacaaggggcaaatataactcttttgatgtcacatttattggctgtttatacacactatgcagcaaaagataaatttgaacgaatggctccggcacttgtgtttttattcatgttagctggacgattggttattgattatcaactgaggcgcaaagaactggcagaaattgctgaacctaaagctcaaaaacaagaatagaaagaaaataacttttcccaaacttagctcatttttattaaaatttttgcacattaattattgtaaggatttgttttaactaaagcttgggaaacacaacccctgttccatccctccaaaataagataacaaatttttgtgttcattcattctagtcatttatgttagtacctatgtaaataatttgtgtgtagggtgtaaattaattcttacatagttttttgtttttttgaccaattaaacatcatgtatattaaaaataaagataataatttaaaacaatgtttacatattatgaatactattataataattagtaattaataactatgaatcttcaccagtggttagtaatttcagtgctttttgtaaatttgcaatggactcagaaacatcatcgtaggttaatgcactagcagcaaacttgcaatatttctgtgctttagtgatttgtgacggcgacagttgtattccatctaaaatgtaaaagaacttttattttcttgtaataaaaattcaacttcacacaattcaaaatgtaccattgttattagactattttggaaataaaatattatctaaccaaaaaaaaaaaaaaaa

SEQ ID NO: 79 - DNA, 1185 bp, Acyrthosiphon pisum
  misc_feature 1..1185: note = Acyrthosiphon pisum protein unc-50 homolog (LOC100161482),
  source 1..1185: mol_type = genomic DNA
atctgtggctatacaaaaaatattatgaaataaatattttataagacaaattaattatgaaaatttatacctttatattatacgtaaatgctgtatgattaatacatttataccgatttaaataaaaataaaatataaatacttcattaatttactttttttttttttaatatttaggtaaataattttatttcaaaatactagcaaatcttaaaatttaatacctacctagttatattgttatttttgaatgtttttcgtataatataatgccaagcagcgtgaattctttccgatcgacatcgtctttgacatatttaccatcaccagtaacacagcgaacatgtatgactgctgctgtgaaacgctaccgctacttgcggaaattatcgaactttaaacaaatggattttgagtttgcactatggcaaatgacgtttttgtttatcaatccacagaaagtatatcgaaactttcaacacagaaaagaaacaaaactacaatttgctcgtgatgaccctgcatttttggtgcttttaagtggacttctatgtgtttcatcaatagcgtttacttacgttctaagacttggtttcgttgaatttttaaaatttctattatatattgtaacagtggattgtttgttatccggtgtcgtaataagttcagtgttatggttagtagccaacagatatttaattaaatccaactacaaaggacaagatgttgaatgggcttatgcatttgatatacatttaaatgcttttgtgccaacattagtaatatctcatatatttcaattgtttttctatcacatatttctcagacacgattggttcttaccattatttattggcaacacactatggcttatagctattggttattacctgtatataacatttttaggatacagctgtttacccatcttgcaaaaaactgaagtattgttaactccatttattctactattttttttatatatattgtcgtttgcaataaattggaatataagcgacaatgtcatgtccatatacaaatacagaattttataaaactgaaatatattatattgtattctttttgattctataaaaaaatgtattttttccaattatatgtttatagaaattgtatgtgtatatttataatacatattatgtcaacctaaaaaaaaaaaaaaaa

SEQ ID NO: 80 - DNA, 2795 bp, Acyrthosiphon pisum
  misc_feature 1..2795: note = Acyrthosiphon pisum protein disulfide-isomerase TMX3 (LOC100158782), transcript variant X2
  source 1..2795: mol_type = genomic DNA
cagttaatacctatgtagtatgtacgatgtacctactaagttgaacttttctatttccacttattagttaattattatgtcaatactcaatactcaacggtgatctgttatgtacttatgtaacgtttttacttaaagttcagttttaaattattctactaatgcatacattttttataactatctatataatttgagaaatatgtgtaatctgaaaatgtctgccaaatgcaattgactttaaaatatactaaacaatatttagttttgaacttagtagtataatagtataataaacaatacatttatatacaactttttacaagagaaatatagtacaaaaatgatgatctctaatatattaactacaactctttccgtgggttttattgtatctgttattctactaaaagacgtacgttcatcaagagtattggaacttagtgataagttcattgaaatgaaacacgatggacattggtttataatgttttatgctccgtggtgtggccattgtaagaggttagaacctatctggcgtcatgttgctcaagcattacaccactctgatataagagttggcaaaatagattgtacaagatataaaagagtagtttcagagtttggattgtctgggtatcctacaataatgttcttcagagatagagaaggtgaatttaaatttaatagtgagcgatctacagaagaaatggttcattttgcaaagcgattggctcgtcctcccgttcaagaggttactgatagtaaagacattgaaatgctcaaacaaacaaataaaaacttctttatgtttattggaaaaagtgttggccatacttgggacctcttttatgatttggcaaaacaatatcaagcacattcttatttttatattacacctgaaataactggaaaacagcattttgtttttggagaaactcctgcagttgtggtgtataaagaagaacaacactactctttcaatgctgaggaatgggatatggacttcaatacaagcttgacacaatggataaacacagaaagatttgatactttcgtaaaagtaactcgatcaaatattcaccatatgttggagaccaacaagtacttagtcttagcattagtggaagagaataaaattgaaaagttacctccgcaacattcagagtttcttcaaatggtagaatcagttattgtgaagaatagagaacgttttcataggaactttcaatttggatggattggttcaccagaattcgccaattctattacttcatcagatgtaaccctaccatcattaatcatttacaattcatctactagacatcatcatttcccagaagaaaaaccacatgaactgacacctgaatcaattactttatttctagaagctgtaagaaatcagtcagtaaaggcatatggaggaagtggtgtatttatacagatgtacaggatgtattatgagacaaagaagtctttagtaaacatctgggttaacaatcctgcagttgctagtgttttatttggattacctatagggttcctactattaatatgctattctacgtgttgcacagacattatggatgctagtgatgatgatgaagatgatgtacatcacgagaagaaagaataagctactcattcaaatattcaaacatattgatttattgactgatgttgaataaataattaaaaacaataaataagacatcaaataattatttatatacttacaaattattcatgtttatatatgtttatatataaacacatacaaaattatttaaattttaatttataaaaatacctaagattacattgagttccaatatataaatcctaccaaattatattgtataaatattaatcctattggctatttgactgtttaatcaattgtgtataatgatctactatatgtaatttattgataaattagatgctgaatttagtattttccagtttgtaaaactttaaatgaatgttgaattttattttaaatgaagggctataaattgtttgttaattttaatcataacttttacaaattattgtaaccacacacatgttttaatactgcagtttatttatcaatataaattttaaccttaagtgttgctttaaaacataaatgcataactagcgcattattattgattttaaatgcttaatttatattacaattattaatttctgaactttccaaattattttttactaagtgaggcttattgttgattggtagatttttttagtaaatcaaatcaaaaatctgtttgaatatagtaaaagttgaaaaaaaataaaagacagtcagaaatttaattaaaataataatggttattttaagttatacaaatcatcctagatgtaaaacagaaacataaataagaacaacaattataatttatatctcgtcattaatatctcaattaactgaaataataaaaataattattagttatatttttaaccgaattacaaatgaaacatgtatatctatccgatgtcaaccattcaatattattcaataagttctataaactccataaaaaatattaaccacatacctattgcattcataatatataatgaactgaaattcatatttgattaaaaattgtatttacctatactgaatagtaacctacaaaaatgtataatctagtcaattttttatttatttaatattctttgtttctgcatgtaaacatttatttgtgttgtaagcttattagttttaatttataacctttgattattgtaaaataaatactagtatattattataa

SEQ ID NO: 81 - protein, 398 aa, Acyrthosiphon pisum
  REGION 1..398: note = resistance to inhibitors of cholinesterase protein 3 isoform X3
MATEISQSKTICILAVVAGCFAILWPSLFYPMLKGSFAPQTHTDNIPQERTPVHLRASKATMHPALMEKGRAIPQPHIGPRHSSPTPHSFKPPIPGSRPTMGGMGGTSNKIAGGDGGGAMNILMPMYTIGILLFFVYTMMKLLSKKDDVEDTIDDKYSSHYPYGRDSKSKLQPQNLPTKLGFIEKDTRDLEIDALRRKLEETEAAMERIVKQMILASECNGMDGNKEFKNENIDEGESPTNTLSNDNVNDTITTKKSTPSEESKSIKSLNTSELYDENTTDSEISVKKIIQVVNMETSESVGGGHCWTPTEKEKIPSKCKTASIEEPISLLIPGMIPKNSQVLISDGPLNTPSETEDHEAVISSKVTLSLIPDDRVEEYLTGDEAEDDNTTSTLPNIN

SEQ ID NO: 82 - protein, 496 aa, Acyrthosiphon pisum
  REGION 1..496: note = uncharacterized protein LOC100159280 isoform X2
MATEISQSKTICILAVVAGCFAILWPSLFYPMLKGSFAPQTHTGCCNVLSSTDVNIVKIVMDICDRVIDTTDRKTKLEKCRHDIFNTCAINITDLINENKVGKISHNKHLIDEIRSLNGSLCLKYHYGVSLTSLGVTHRLKESDSLMNNIPQERTPVHLRASKATMHPALMEKGRAIPQPHIGPRHSSPTPHSFKPPIPGSRPTMGGMGGTSNKIAGGDGGGAMNILMPMYTIGILLFFVYTMMKLLSKKDDVEDTIDDKYSSHYPYGRDSKSKLQPQNLPTKLGFIEKDTIVNAFSSLLEDVNREILSSIVEKENVPTIIENEEFKNENIDEGESPTNTLSNDNVNDTITTKKSTPSEESKSIKSLNTSELYDENTTDSEISVKKIIQVVNMETSESVGGGHCWTPTEKEKIPSKCKTASIEEPISLLIPGMIPKNSQVLISDGPLNTPSETEDHEAVISSKVTLSLIPDDRVEEYLTGDEAEDDNTTSTLPNIN

SEQ ID NO: 83 - protein, 502 aa, Acyrthosiphon pisum
  REGION 1..502: note = uncharacterized protein LOC100159280 isoform X1
MATEISQSKTICILAVVAGCFAILWPSLFYPMLKGSFAPQTHTGCCNVLSSTDVNIVKIVMDICDRVIDTTDRKTKLEKCRHDIFNTCAINITDLINENKVGKISHNKHLIDEIRSLNGSLCLKYHYGVSLTSLGVTHRLKESDSLMNNIPQERTPVHLRASKATMHPALMEKGRAIPQPHIGPRHSSPTPHSFKPPIPGSRPTMGGMGGTSNKIAGGDGGGAMNILMPMYTIGILLFFVYTMMKLLSKKDDVEDTIDDKYSSHYPYGRDSKSKLQPQNLPTKLGFIEKDTRDLEIDALRRKLEETEAAMERIVKQMILASECNGMDGNKEFKNENIDEGESPTNTLSNDNVNDTITTKKSTPSEESKSIKSLNTSELYDENTTDSEISVKKIIQVVNMETSESVGGGHCWTPTEKEKIPSKCKTASIEEPISLLIPGMIPKNSQVLISDGPLNTPSETEDHEAVISSKVTLSLIPDDRVEEYLTGDEAEDDNTTSTLPNIN

SEQ ID NO: 84 - protein, 144 aa, Acyrthosiphon pisum
  REGION 1..144: note = uncharacterized protein LOC100164060 precursor
MIRCQMYAMVVFAFGCLVLLPDYGKAINCWVCSTDTDQRCNDPMNMTKGAIEDCSRAPHSAFLQPVCKKQKQRVNGELLIIRSCAWASDSRSDDGPCAINTPANIRIEHCSTCDQDLCNGAVVIGGCTVFLTMVLAVAVHLATY

SEQ ID NO: 85 - protein, 162 aa, Acyrthosiphon pisum
  REGION 1..162: note = protein quiver
MNGAKCAACLAAVLFVLFAFVVKGAKSIICYKCNSKYDPRCGDPFNPYTIGLVDCNITDVPSHLKGKEPVICRKTVQNIHKSIRIVRDCGYVDDPAHDDQDCYGRLGSHDIDVTHCSCTTDMCNGSKDVVSNGPYWYSATATAMVATAAVTIMGFFLNIHHL

SEQ ID NO: 86 - protein, 148 aa, Acyrthosiphon pisum
  REGION 1..148: note = uncharacterized protein LOC100164124
MMPSALCVFCMSFIVLYSVYSVCGLMCWHCDSLHDPKCVDPFDNTSMPMKDCKLEKLDTYPGVRGTMCRKIRQKVYGKWRFYRSCAFLGEPGIGGDERYCLMRTGTHNIFTEYCTCNSRDGCNGASGISSSLLSISLAPALIVYRRLL

SEQ ID NO: 87 - protein, 156 aa, Acyrthosiphon pisum
  REGION 1..156: note = uncharacterized protein LOC100160953 precursor
MSCTVTSVAYATIALIAVSIVFNTHTVKAQAEIRCYVCNSHNDTDCAQEKLPDHFKTECSKLNIGVPEDSRKNYTLCRKIIQTIEPEVNGLPSDRNRIIRTCGWDASNYNNKCYHRSGFGGKQDVCTCMTDYCNSAQKTYIAFVTVVAAAFAILIL

SEQ ID NO: 88 - protein, 143 aa, Acyrthosiphon pisum
  REGION 1..143: note = protein quiver precursor
MRWTIAVVVLVNISLSHEECFPPRSIYCYECDSAKDPRCKDPFNYTAMPQDQPPLVSCNGCCVKMVRYVRTSYESIKRTCTSQLQINLFMVDHVCMMEGTGTGHMCFCEEDNCNAATGPARSSALVSGAIALAMYALKLLLYR

SEQ ID NO: 89 - protein, 152 aa, Acyrthosiphon pisum
  REGION 1..152: note = transmembrane protein 35
MGSIVLKSLSVLLGIFFLFVGTLKMSPVISKELHKDLRKDYVKYAKVFPLSKMIDFKVPAKWYRRAVGGTEVLSGVCLAFVPYRNVKQGANITLLMSHLLAVYTHYAAKDKFERMAPALVFLFMLAGRLVIDYQLRRKELAEIAEPKAQKQE

SEQ ID NO: 90 - protein, 261 aa, Acyrthosiphon pisum
  REGION 1..261: note = protein unc-50 homolog
MPSSVNSFRSTSSLTYLPSPVTQRTCMTAAVKRYRYLRKLSNFKQMDFEFALWQMTFLFINPQKVYRNFQHRKETKLQFARDDPAFLVLLSGLLCVSSIAFTYVLRLGFVEFLKFLLYIVTVDCLLSGVVISSVLWLVANRYLIKSNYKGQDVEWAYAFDIHLNAFVPTLVISHIFQLFFYHIFLRHDWFLPLFIGNTLWLIAIGYYLYITFLGYSCLPILQKTEVLLTPFILLFFLYILSFAINWNISDNVMSIYKYRIL

SEQ ID NO: 91 - protein, 430 aa, Acyrthosiphon pisum
  REGION 1..430: note = protein disulfide-isomerase TMX3 isoform X2
MMISNILTTTLSVGFIVSVILLKDVRSSRVLELSDKFIEMKHDGHWFIMFYAPWCGHCKRLEPIWRHVAQALHHSDIRVGKIDCTRYKRVVSEFGLSGYPTIMFFRDREGEFKFNSERSTEEMVHFAKRLARPPVQEVTDSKDIEMLKQTNKNFFMFIGKSVGHTWDLFYDLAKQYQAHSYFYITPEITGKQHFVFGETPAVVVYKEEQHYSFNAEEWDMDFNTSLTQWINTERFDTFVKVTRSNIHHMLETNKYLVLALVEENKIEKLPPQHSEFLQMVESVIVKNRERFHRNFQFGWIGSPEFANSITSSDVTLPSLIIYNSSTRHHHFPEEKPHELTPESITLFLEAVRNQSVKAYGGSGVFIQMYRMYYETKKSLVNIWVNNPAVASVLFGLPIGFLLLICYSTCCTDIMDASDDDEDDVHHEKKE